(19) United States
(12) Patent Application Publication
(10) Pub. No.: US 2019/0209006 A1
(43) Pub. Date: Jul. 11, 2019

(54) SEGMENTATION-BASED CORNEAL MAPPING

(71) Applicant: University of Miami, Miami, FL (US)

(72) Inventors: Mohamed Abou Shousha, Pembroke Pines, FL (US); Amr Saad Mohamed Elsawy, Miami, FL (US); Marco Ruggeri, Miami, FL (US)

(73) Assignee: University of Miami, Miami, FL (US)

(21) Appl. No.: 16/269,549

(22) Filed: Feb. 6, 2019

Related U.S. Application Data
(63) Continuation-in-part of application No. 15/868,856, filed on Jan. 11, 2018.
(60) Provisional application No. 62/445,106, filed on Jan. 11, 2017.
(60) Provisional application No. 62/627,189, filed on Feb. 6, 2018.

(51) Int. Cl. A61B 3/107; G06T 7/00; A61B 3/10; A61B 3/103

(52) U.S. Cl. CPC A61B 3/107; G06T 7/0012; A61B 3/102; G06T 7/60; A61B 3/103; G06T 2207/10101; A61B 3/1005

(57) ABSTRACT

In some embodiments, images of a cornea may be obtained, and the images of the cornea may be segmented to detect a tear film layer of the cornea and an epithelium layer of the cornea. Thickness of the tear film layer and thickness of the epithelium layer may be determined based on the segmentation of the images of the cornea. A thickness map may be generated based on the thickness of the tear film layer and the thickness of the epithelium layer. As an example, the thickness map may comprise visual differences in thickness across the tear film layer and the epithelium layer. In some embodiments, the foregoing may be performed with respect to one or more other microlayers of the cornea in addition to or alternatively to the tear film layer or the epithelium layer.

### RELATED APPLICATIONS

This application is a continuation-in-part of U.S. patent application Ser. No. 15/868,856, filed Jan. 11, 2018, which claims the benefit of U.S. Provisional Application No. 62/445,106, filed Jan. 11, 2017. This application also claims the benefit of U.S. Provisional Application No. 62/627,189, filed Feb. 6, 2018. Each of the foregoing applications is hereby incorporated by reference in its entirety.

### STATEMENT OF GOVERNMENT SUPPORT

This invention was made with government support under Grant No. K23EV026118 awarded by the National Eye Institute. The Government has certain rights in the invention.

### FIELD OF THE INVENTION

The present disclosure relates to corneal scanning or mapping, including, for example, scanning of a cornea (e.g., scanning of container-sealed a cornea of a donor, scanning of a cornea of a patient, etc.), segmentation of one or more images of a cornea, generation of a corneal-related map via such segmentation or other techniques, or determinations derived from such segmentation and mapping.

### BACKGROUND

Although scanning and diagnostic systems for detecting eye conditions exist, many such systems involve invasive procedures and/or tests that are not representative of patient symptoms, including such systems to scan or analyze characteristics of aqueous deficiency, evaporative dry eye syndrome (DES), corneal ectasia, corneal limbal stem cell deficiency, keratoplasty graft rejection episode or failure, Fuchs' dystrophy, or other conditions. As an example, while confocal microscopy can be used to diagnose DES, it is a time-consuming procedure that requires contact with the ocular surface, making it difficult to incorporate into everyday clinics and limits its use as a research tool. Tear film osmolarity is another technique used to diagnose DES, but it is also invasive and time consuming.

In addition, given the large number of corneal transplants performed annually, it is important to non-invasively detect eye conditions of a donor cornea (e.g., Fuchs' dystrophy or other conditions) prior to transplanting the cornea. Slit-lamp examination is one technique used to detect such eye conditions, but this technique offers only limited magnification and often misses detection of potential subclinical rejection episodes. Other techniques (e.g., endothelial cell count using specular microscopy, central cornea thickness measurements, etc.) lack sufficient reproducibility or sensitivity, limiting their usefulness in the diagnosis of mild cases. The wide range of normal corneal thickness also complicates their usefulness for diagnosis of mild cases (e.g., mild corneal graft rejection, edema, ectasia, etc.). As an example, there is significant overlap between normal thin corneas and early ectasia patients, making it difficult to ectasia in its early stages. These and other drawbacks exist.

### SUMMARY

In some embodiments, images of a cornea may be obtained, and the images of the cornea may be segmented to detect a tear film layer of the cornea and an epithelium layer of the cornea. Thickness of the tear film layer and thickness of the epithelium layer may be determined based on the segmentation of the images of the cornea. A thickness map may be generated based on the thickness of the tear film layer and the thickness of the epithelium layer. As an example, the thickness map may comprise visual differences in thickness across the tear film layer and the epithelium layer. In some embodiments, the foregoing may be performed with respect to one or more other microlayers of the cornea in addition to or alternatively to the tear film layer or the epithelium layer. It should be noted that, although some embodiments describe specific types of maps (e.g., heat map, bullseye map, etc.), other types of maps may be generated or used to represent characteristics of a cornea (e.g., its microlayers or other portions of the cornea) or other tissue.

In some embodiments, the cornea may be in a container, and the image of the cornea may be obtained via an imaging device outside the container while the cornea is in the container. In some embodiments, a reference arm of the imaging device may be adjusted to position a zero delay line posterior to the cornea, and the image of the cornea may be obtained via the imaging device based on the adjustment of the reference arm of the imaging device (e.g., while the cornea is in the container).

Various other aspects, features, and advantages of the invention will be apparent through the detailed description of the invention and the drawings attached hereto. It is also to be understood that both the foregoing general description and the following detailed description are exemplary and not restrictive of the scope of the invention. As used in the specification and in the claims, the singular forms of “a,” “an,” and “the” include plural referents unless the context clearly dictates otherwise. In addition, as used in the specification and the claims, the term “or” means “and/or” unless the context clearly dictates otherwise.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1 illustrates a process for evaluating the eye of a subject, in accordance with an embodiment.

FIGS. 2A and 2B illustrate maps developed by the process of FIG. 1 for a subject with healthy corneal tissue. FIG. 2A illustrates a heat map of healthy corneal tissue. FIG. 2B illustrates a bullseye map of healthy corneal tissue.

FIGS. 3A and 3B illustrate maps developed by the process of FIG. 1 for a subject with keratoconus. FIG. 3A illustrates a heat map of a keratoconus condition. FIG. 3B illustrates a bullseye map of the keratoconus condition.

FIG. 4 illustrates an example process of segmentation and microlayer identification and thickness determination, in accordance with an embodiment.

FIG. 5A is a cross-sectional image of a first raw high-resolution image of a cornea, in accordance with an embodiment.

FIG. 5B is a cross-sectional image of registered and averaged images of the cornea, in accordance with an embodiment.

FIG. 6 is a cross-sectional image of an entire cornea with microlayers mapped out and demarcated by their respective anterior surfaces for each layer. EP1 is the anterior surface of the corneal epithelium; EP2 is the interface between the basal epithelium and the remaining layers of the epithelium; BW1 is the interface between the basal epithelium and the Bowman's layer; BW2 is the interface between the Bowman's layer and the stroma; DM is the interface between the stroma and the Endothelial/Descemet's complex layer and the stroma; and EN is the posterior interface of the Endothelial/Descemet's complex layer.

FIG. 7 is a schematic illustration of an example optical imaging system for performing thickness mapping of corneal microlayers in performing the processes of FIGS. 1 and 4, in accordance with an embodiment.

FIG. 8 illustrates another process for evaluating the eye of a subject, in accordance with an embodiment.

FIG. 9 illustrates a registration process that may be performed during the process of FIG. 8, in accordance with an embodiment.

FIG. 10 illustrates a segmentation process that may be performed during the process of FIG. 8, in accordance with an embodiment.

FIG. 11 illustrates a legend for a bullseye mapping thickness map, in accordance with an embodiment.

FIG. 12A illustrates a heat map of a Bowman's layer and a bullseye map using the mapping schema of FIG. 11, for a normal, healthy subject, in accordance with an embodiment.

FIG. 12B illustrates a similar heat map of Bowman's layer and a bullseye map, for a subject with keratoconus, in accordance with an embodiment.

FIGS. 13A and 13B illustrate a refinement procedure during segmentation identifying an anterior boundary of an epithelial layer, with FIG. 13A showing a segmentation line prior to refinement, and FIG. 13B showing the segmentation line after refinement, in accordance with an embodiment.

FIGS. 14A and 14B illustrate a refinement procedure during segmentation identifying an anterior boundary of a Bowman's layer, with FIG. 14A showing a segmentation line prior to refinement, and FIG. 14B showing the segmentation line after refinement, in accordance with an embodiment.

FIG. 15A illustrates a heat map showing the depth of a collagen crosslinking microlayer within the cornea measured from the epithelium, in accordance with an embodiment.

FIG. 15B illustrates a thickness heat map of a collagen crosslinking microlayer within the cornea, in accordance with an embodiment.

FIG. 15C illustrates a heat map showing a distance between a collagen crosslinking microlayer within the cornea and the endothelium, in accordance with an embodiment.

FIG. 16 illustrates a procedure for processing images to segment the epithelium and tear film layers, in accordance with an embodiment.

FIG. 17 illustrates a flattening of a composite image using the anterior surface, in accordance with an embodiment.

FIG. 18 illustrates a vertical projection of a flattened image using the anterior surface, in accordance with an embodiment.

FIG. 19 illustrates the vertical projection of FIG. 18 overlaid on the image illustrating correspondence of the peaks and valleys, in accordance with an embodiment.

FIG. 20 illustrates a segmentation of the corneal layers that includes the tear film (TF), epithelium (EP), basal-epithelium (BS), Bowman's Layer (BW), stroma (ST), Descemet's membrane (DM), and endothelium layer (EN), in accordance with an embodiment.

FIG. 21A illustrates a heat map of an epithelium layer, in accordance with an embodiment.

FIGS. 21B-21D illustrate bullseye maps of an epithelium layer, in accordance with an embodiment.

FIG. 22A illustrates a heat map of a tear film layer, in accordance with an embodiment.

FIGS. 22B-22D illustrate bullseye maps of an epithelium layer, in accordance with an embodiment.

FIG. 23 illustrates an example vertical projection overlaid on a corneal image showing limited separation of the tear film and epithelium corresponding to a thickened white band, in accordance with an embodiment.

FIGS. 24A-24B illustrate a horizontal gradient of corneal image and the absolute of the horizontal gradient, in accordance with an embodiment.

FIGS. 24C-24D illustrate a vertical gradient of corneal image and the absolute of the vertical gradient, in accordance with an embodiment.

FIGS. 24E-24F illustrate a weighted sum of the gradient absolutes and the locally normalized gradient, in accordance with an embodiment.

FIGS. 25A-25B illustrate a 5-connectivity neighborhood and a related directed graph constructed for a corneal image, in accordance with an embodiment.

FIGS. 26A-26B illustrate a gradient of a corneal image augmented with artifacts and the gradient with further augmentations, in accordance with an embodiment.

FIGS. 26C-26D illustrate an initial segmentation of a gradient of a corneal image and a corrected segmentation of the gradient, in accordance with an embodiment.

FIGS. 27A-27B illustrate a corrected segmentation overlaid on a raw optical coherence topography (OCT) corneal image and zoom-in view that shows a misaligned boundary, in accordance with an embodiment.

FIGS. 28A and 28B illustrate alignment of the segmentation with one or more boundaries, in accordance with an embodiment.

FIGS. 29A-29F illustrate additional examples of segmentations, in accordance with an embodiment.

FIGS. 30A-30B illustrate segmentations resulting from use of a double flattening technique, in accordance with an embodiment.

FIG. 31 illustrates a simulation of the mean of normal cornea thickness, in accordance with an embodiment.

FIGS. 32A-32B illustrate bullseye maps for the mean and standard deviation of normal cornea thickness, respectively, in accordance with an embodiment.

FIG. 33 illustrate a simulation of the mean of abnormal cornea thickness and a simulation of the mean of normal cornea thickness, in accordance with an embodiment.

FIGS. 34A-34B illustrate bullseye maps for the mean and standard deviation of abnormal cornea thickness, respectively, in accordance with an embodiment.

FIGS. 35A-35B illustrate a chart and a corresponding legion that indicate the regional means and standard deviations for normal and abnormal cases, in accordance with an embodiment.

FIGS. 36A-36B illustrates heat and bullseye maps of the thickness difference between normal and abnormal corneas, respectively, in accordance with an embodiment.

FIG. 37 illustrates an imaging device for obtaining images of a cornea, in accordance with an embodiment.

FIG. 38A-38C illustrates high-definition OCT images from a donor graft, a control eye, and a Fuchs' endothelial corneal dystrophy eye, respectively in accordance with an embodiment.

### DETAILED DESCRIPTION

In the following description, for the purposes of explanation, numerous specific details are set forth in order to provide a thorough understanding of the embodiments of the invention. It will be appreciated, however, by those having skill in the art that the embodiments of the invention may be practiced without these specific details or with an equivalent arrangement. In other instances, well-known structures and devices are shown in block diagram form in order to avoid unnecessarily obscuring the embodiments of the invention.

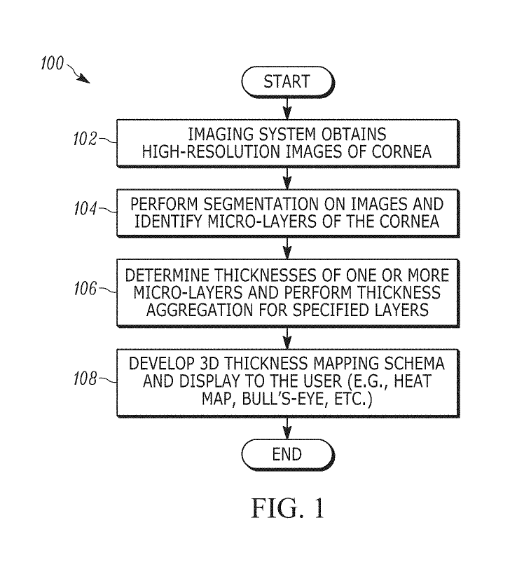

FIG. 1 illustrates a computer-implemented method 100 of evaluating the eye of a subject, in accordance with an embodiment. In some embodiments, the method 100 is adapted to evaluate corneal conditions, including keratoconus, pellucid marginal degeneration, post-refractive surgery ectasia, keratoglobus, corneal transplant rejection and corneal transplant failed grafts, Fuchs' dystrophy, corneal limbal stem cell deficiency, and dry eye syndrome (DES).

The method 100 may be implemented by a system, such as that described further below in reference to FIG. 7. In the illustrated example, at operation 102, an optical imaging system obtains a plurality of high-resolution images of a cornea of the eye(s) of a subject. As an example, the high-resolution images may be captured in real-time. In other examples, the images may be previously-collected corneal images stored in an image database or other memory.

Whether the optical imaging system itself records the corneal images directly from the subject or whether the corneal images are obtained from another imager or from a database, the image processing, analysis, and diagnostic techniques herein may be implemented partly or wholly within an existing optical imaging system or partly or wholly within a dedicated image processor. Example optical imaging systems include suitable corneal imagers such as charge-coupled device (CCD) cameras, corneal topography scanners using optical slit designs, such as the Orbscan system (Bausch & Lomb, Rochester, N.Y.), Scheimpflug imagers such as the Pentacam (Oculus, Lynnwood, Wash.), conventional microscopes collecting reflected light, confocal microscope-based systems using a pinhole source of light and conjugate pinhole detector, optical coherence tomography (OCT) imagers imaging the cornea and anterior segment imagers, optical interferometry-based systems in which the light source is split into the reference and measurement beams for corneal image reconstruction, and high-frequency high-resolution ultrasound biomicroscopy (UBM) imagers.

The corneal images may be a plurality of images each captured with the eye looking in a different direction, from which a wide scan of the cornea is formed by stitching images together. In some examples, the images are a plurality of wide scan images of the cornea collected from a wide-angle optical imaging system, where the wide-angled images are corrected for optical distortion, either through image processing or through a corrective optic stage in the imaging system.

In examples, the obtained images contain images of one or more biologically-definable microlayers of the cornea. Such images would typically be un-segmented, raw cornea image data, meaning that the microlayers would not be identified directly in the images, but rather the images would capture one or more microlayers that are segmented by the imaging system applying the unique algorithm techniques herein.

In one example, raw images including one or more biologically-defined microlayers of the cornea may be obtained by the imaging system. Utilizing one or more of the image processing algorithms described herein, the imaging system may segment one or more of the biologically-defined microlayers from the obtained images.

At operation 104, the method 100 performs a segmentation process on the plurality of high-resolution images. As an example, an image processor identifies, via the segmenting the images, one or more of a plurality of biologically-defined microlayers of the cornea. From the segmentation, the image processor determines the thickness for the one or more biologically-defined microlayers of the cornea. The image processor, also referred to herein as an imaging system or machine, may be a processor of an existing optical imaging system, such as an OCT imager, while in some examples, that image processor is in a separate system that receives the high-resolution images from the optical imaging system. The image processor may be implemented on a general-purpose processor or on a dedicated processor, by way of example.

The image processor, at operation 104, may be programmed to identify each of the plurality of biologically-defined microlayers, e.g., an epithelium, a basal epithelial layer, a Bowman's layer, and one or more endothelial/Descemet's layers complex. In other examples, such as when the imaging system is programmed to identify for a particular corneal condition, the image processor, at operation 104, may segment the images and identify the thickness of only certain microlayers of the cornea.

In another example, image processor, at operation 104, may be programmed to identify one or more of the biologically-defined microlayers, which may include, for example, an epithelium, a tear film, an epithelium layer separates from a tear film, a basal epithelial layer, a Bowman's layer, or one or more endothelial/Descemet's layers complex. In some examples, the imaging system may be programmed to identify one or more corneal conditions, wherein, at operation 104, the image processor may segment the images and identify the thickness or topology of only certain microlayers of the cornea relevant to the corneal condition (or conditions) the system is to identify.

With the microlayers segmented into different image data, and the thicknesses of the microlayers determined at operation 104, at operation 106, the image processor combines the image data for each of the plurality of biologically-defined microlayers of the cornea and produces a thickness map of the total corneal thickness (whole cornea from one limbus to the other). That is, in some examples, the thickness map is a summation of the determined thicknesses for each of the plurality of biologically-defined microlayers, illustrating collective thickness across the cornea, e.g., providing a three-dimensional map of the whole cornea from one limbus to the other. Further, the combinational data in the thickness map may retain specified thickness values for each of the microlayers. That is, the techniques measure the thickness of the corneal microlayers across the entire cornea from one end to the other. The ability to determine thickness across the cornea allows for measuring regions of abnormal or irregular thickness across the entire cornea. But the determinations at operations 104 and 106 also allow the image processor to analyze microlayer thicknesses as well, thus providing two levels of thickness analysis, a first overall corneal thickness and a second, microlayer thickness.

In the illustrated example, at operation 108, the imaging system develops a thickness map and displays the thickness map through a monitor (or display) via operation 108. That thickness map may visually identify differences in thickness of corneal microlayers across the thickness map, by visually depicting the overall corneal microlayer thicknesses. The visual depiction identifies differences in thicknesses that are correlated to diagnosable conditions of the cornea.

In some embodiments, the imaging system may develop a mapping schema (e.g., a three-dimensional mapping schema or other mapping schema). The mapping scheme may include a thickness map for one or more of the corneal layers. The thickness map may include a map of the whole cornea, particular corneal layers, such as adjacent layers, or a single corneal layer across the entire cornea or portion thereof. The system may delineate, as a result of the segmentation and determinations at operations 104 and 106, layers within the thicknesses map from which the mapping schema may be developed for display to a user in operation 108. As described herein, the system may be programmed to develop specific or specified mapping schema for display to a user. For example, the image data of one or more layers may be extracted or analyzed to develop a three-dimensional mapping schema from which a diagnosable condition may be assessed with respect to the cornea imaged. Developing the three-dimensional mapping schema may include transforming the image thickness data with respect to one or more of the segmented layers into a thickness map comprising a visual depiction related to thickness, e.g., differences in thicknesses across one or more layers or sections thereof, that may correlate to a diagnosable condition of the cornea, such as a specific diagnosable condition. In various embodiments, thickness maps may be developed that include depictions of thickness or surface topology. In further embodiments, and as described in more detail below, the depictions may include minimums, maximums, indices, ratios, deviations, or differences within a layer, such as with respect to other regions of a layer, or with respect to normative data or thresholds, for example. As also described in more detail below, the system may develop a three-dimensional mapping schema that includes generating a thickness map, which may or may not be displayed, and compare the generated thickness map to a normal or control thickness map or thickness map exemplary of a condition. In some examples, the generated thickness map may be compared to thresholds or previously generated thickness maps of the cornea to track progression or stability. The system may generate a thickness map based on such comparisons depicting minimums, maximums, indices, ratios, deviations, differences, etc., which may correspond to a diagnosable condition or be used to further a diagnosis.

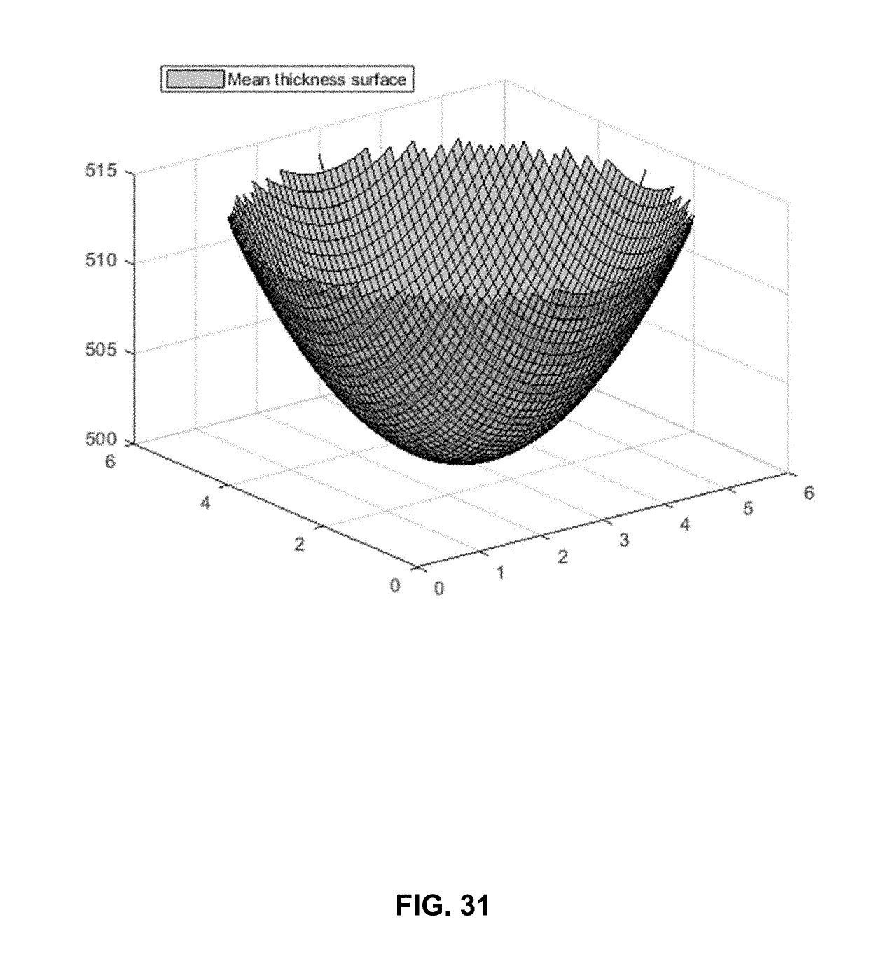

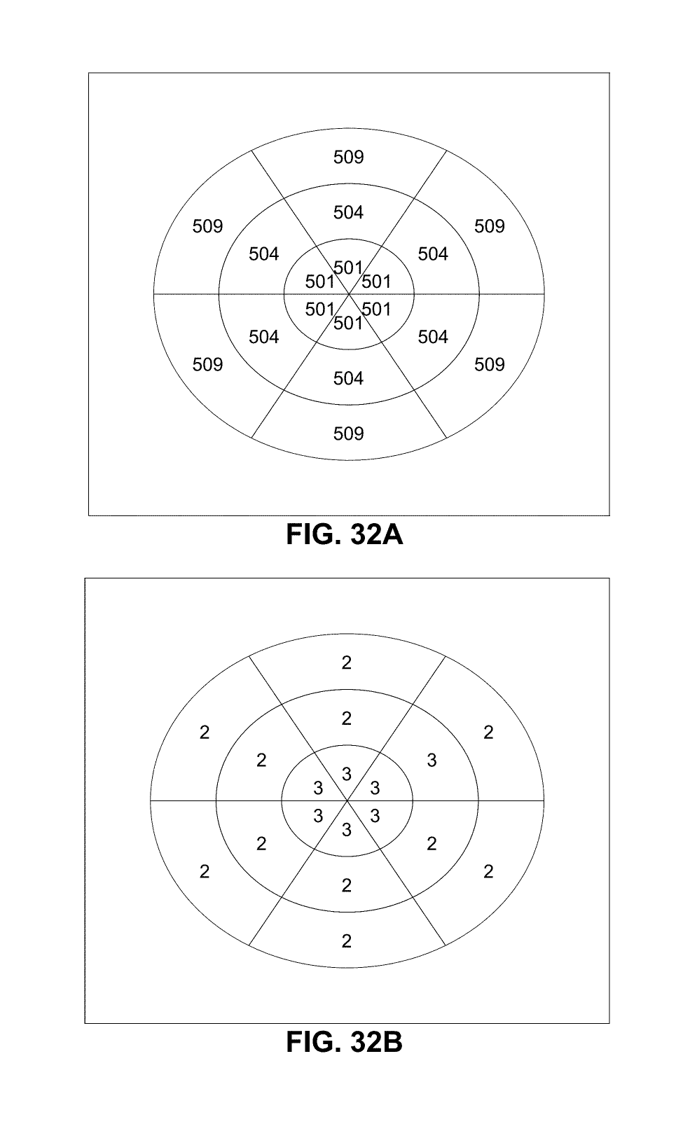

As an example, FIG. 31 shows a simulation of the mean of normal cornea thickness (green surface) based on normative data (e.g., stored in one or more databases). As shown in FIG. 31, the thickness increases slowly towards the periphery, and the elevation of the apex is about 500 mm at the apex. FIGS. 32A and 32B shows bullseye maps for the mean and standard deviation of normal cornea thickness, respectively. The standard deviation bullseye map for the normal case may, for example, represent the regional mean in the standard deviation bullseye map for the normal case.

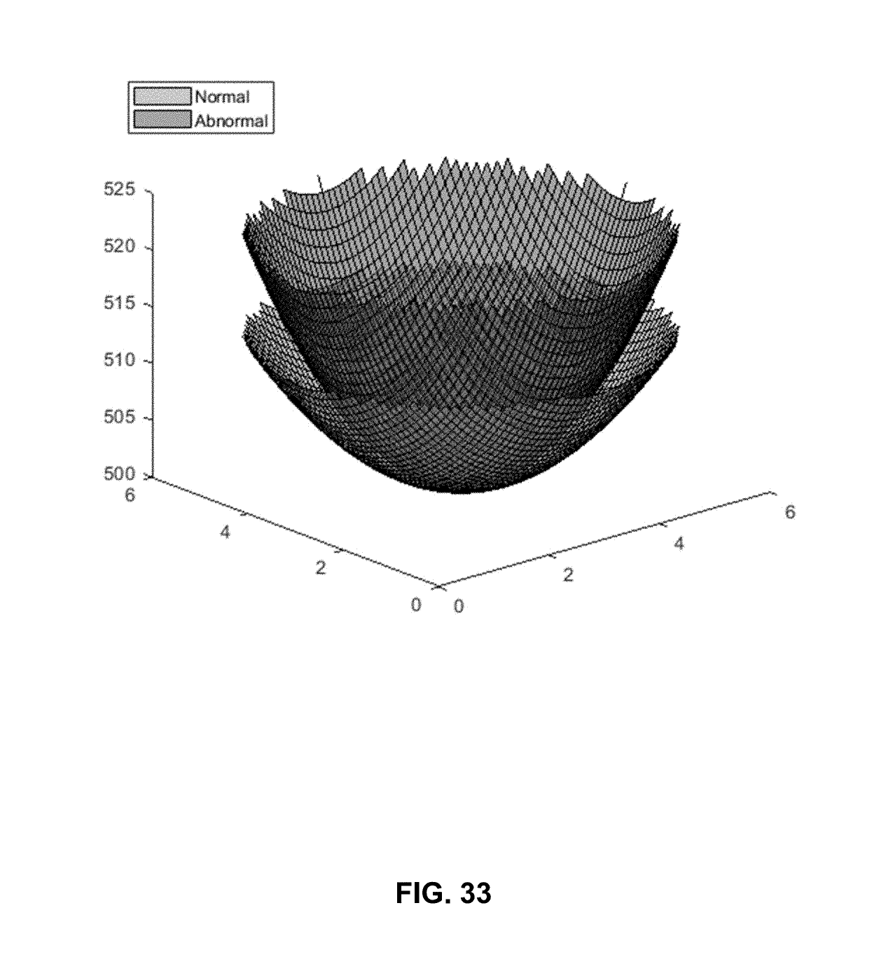

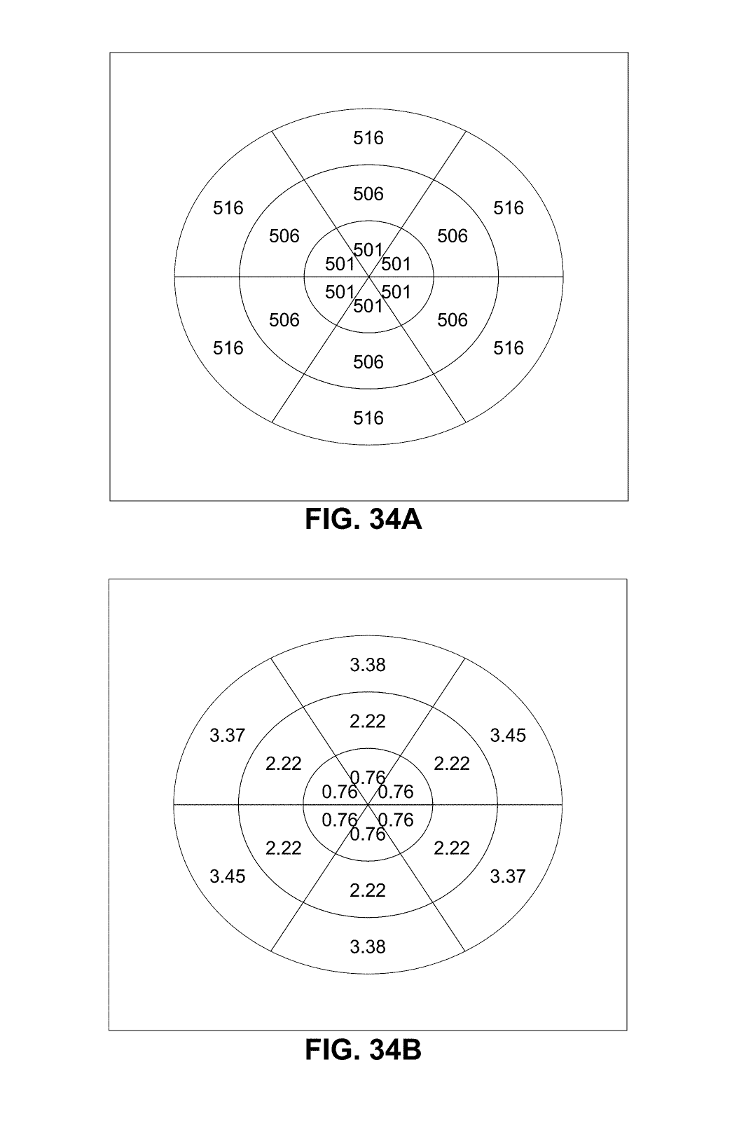

As another example, FIG. 33 shows a simulation of the mean of abnormal cornea thickness (red surface) and a simulation of the mean of normal cornea thickness (green surface). FIGS. 34A and 34B shows bullseye maps for the mean and standard deviation of abnormal cornea thickness, respectively. The standard deviation bullseye map for the abnormal case may, for example, represent the regional variation in the mean map for the abnormal case.

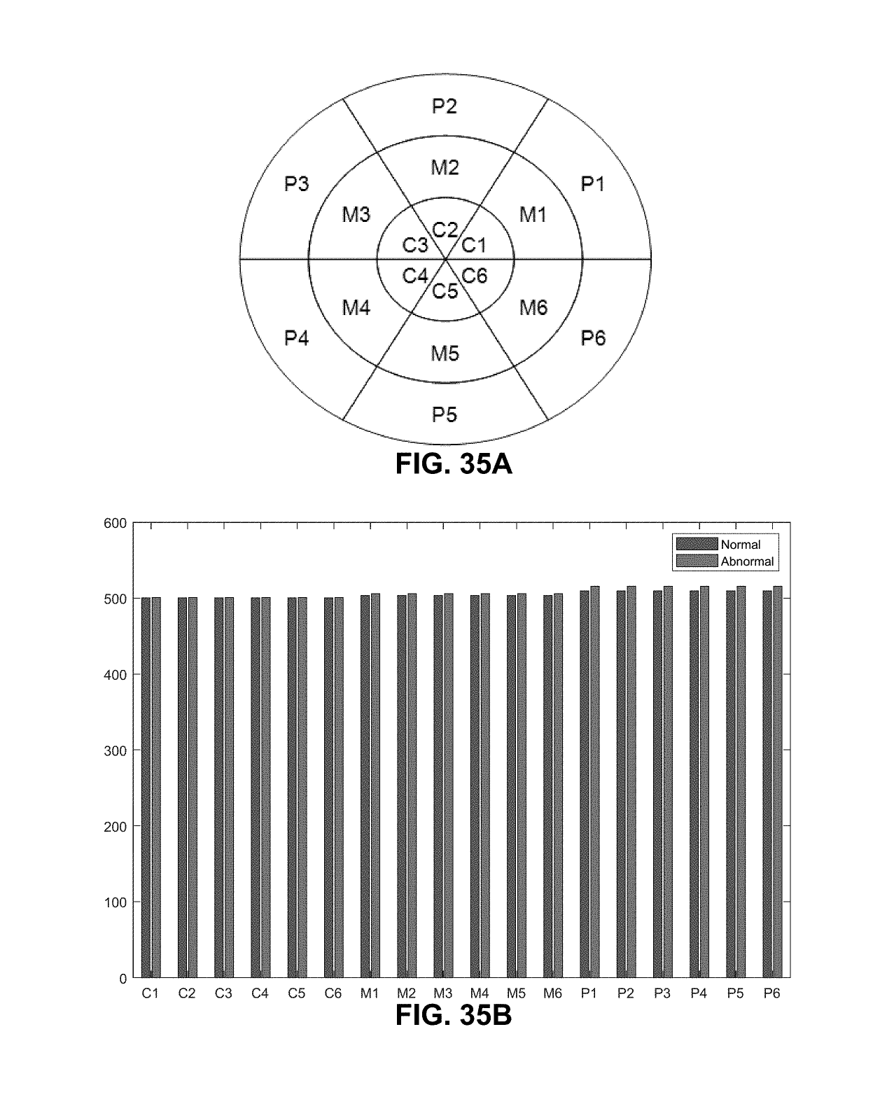

As another example, FIG. 35A shows a legion for the region labels in the chart shown in FIG. 35B. As indicated in FIG. 35A, the labels C1-C6 represent central regions of the cornea, the labels M1-M6 represent middle regions of the cornea, and the labels P1-P6 represent peripheral regions of the cornea. FIG. 35B shows a chart indicating the regional means and standard deviations for the normal and abnormal cases. As shown in the chart in FIG. 35B, an increase in the thickness is more noticeable in the peripheral regions.

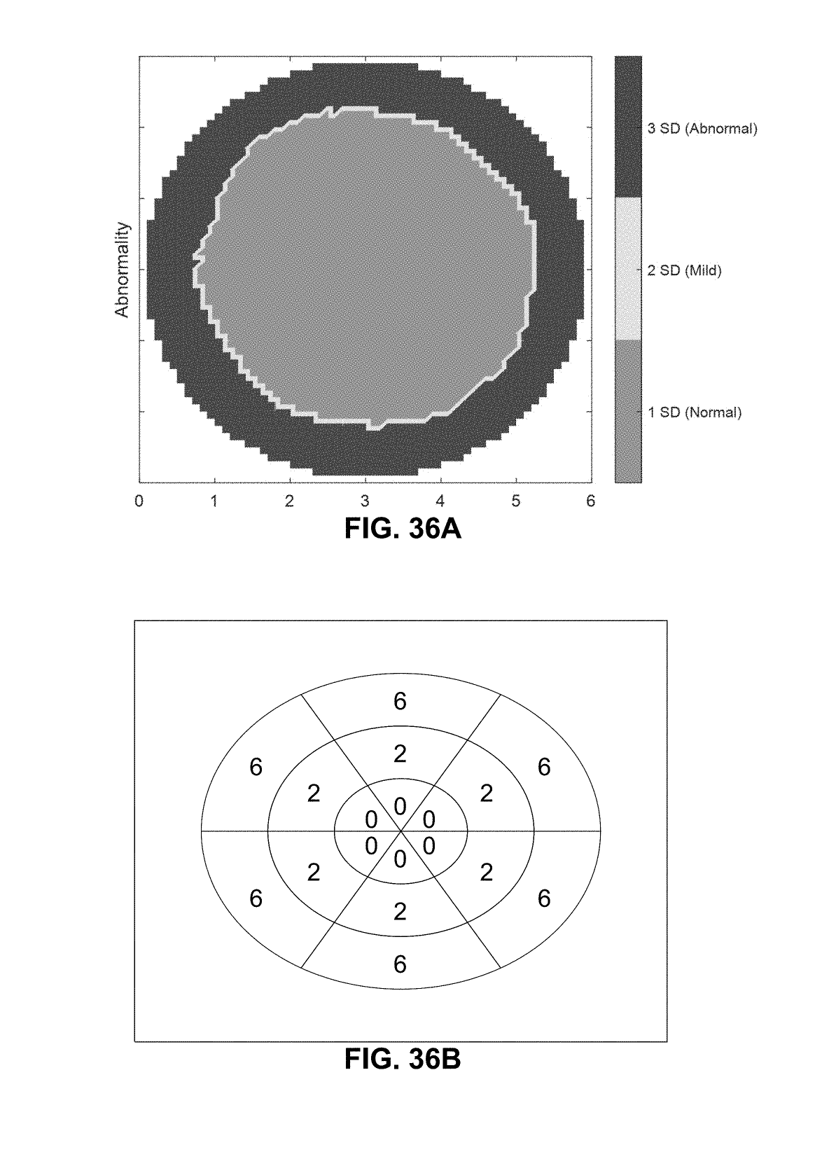

As another example, FIGS. 36A and 36B shows heat and bullseye maps of the thickness difference between normal and abnormal corneas, respectively. In one use case, with respect to FIG. 36A, the green regions represent the thickness difference values that are within 1 standard deviation of normal cornea thickness (e.g., a mean of normal cornea thickness), the yellow region represents the thickness difference values that are within 1-2 standard deviation of normal cornea thickness, and the red region represents the thickness difference values that are within 2-3 standard deviation of normal cornea thickness. FIG. 36B shows the bullseye map the thickness difference (e.g., represented by the heatmap map of FIG. 36A).

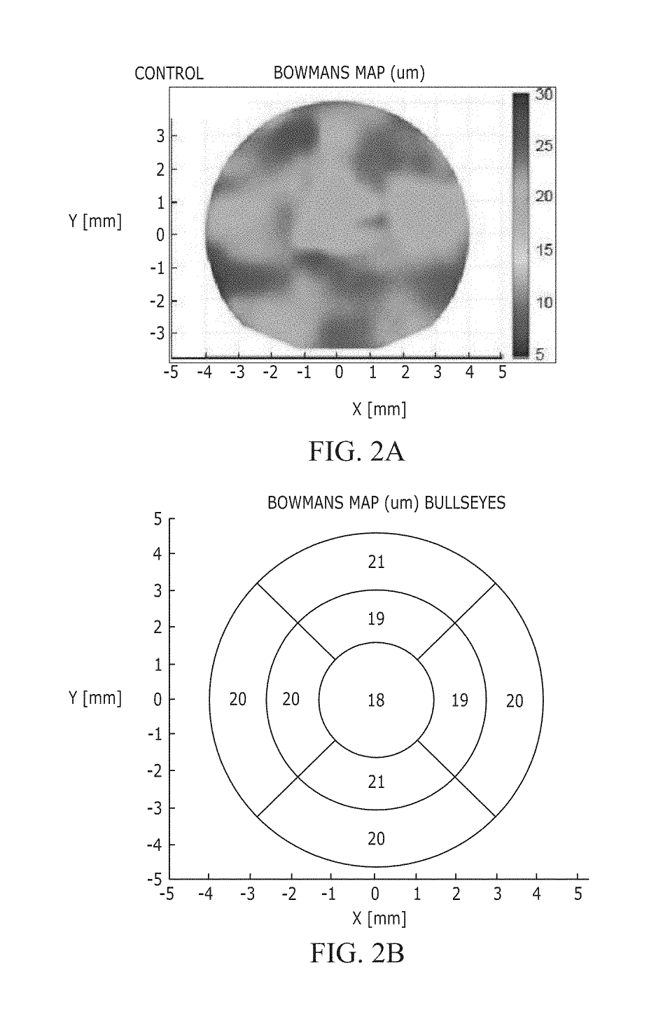

FIG. 2A illustrates example thickness map depiction of a corneal microlayer, specifically a heat map of the Bowman's layer. The heat map shows variations in color as coded by color or shading. The heat map legend is based on the obtained normative thickness data of corneal microlayers, using green for normal, and yellow for borderline, and red for pathology. As the thinning of the basal epithelial and Bowman's layer is pathological, red may be used to represent the pathological thinning, yellow may be used to represent the borderline thinning, and green may be used to represent the normal range thickness. In Endothelial/Descemet's layers complex, thickening of the layer is the pathological change. Thus, red may be used to represent the pathological thickening based on the normative data obtained, yellow may be used to represent borderline thickness, and green may be used to represent normal thickness.

In the illustrated example, the thickness values were determined to extend from at or about 5 microns to at or about 30 microns over the entire Bowman's layer of the cornea. FIG. 2B illustrates another three-dimensional thickness map of the same Bowman's layer, but in the form of a bullseye thickness map.

The heat map and bullseye maps are examples of different three-dimensional thickness map schemas that may be generated through the present techniques. Further, as discussed herein, for each of these types of thickness map schemas there are numerous variants. As an example, a bullseye map may illustrate values for the thicknesses of a microlayer or that bullseye map may illustrate ratios of thicknesses between regions of a microlayer. The bullseye map displays the thickness map of the Bowman's layer as a series of thickness values for 9 sections of the layer: one central region centered around the pupil, and eight wedge shaped regions extending radially outward from the central region. The bullseye map can be presented in different mapping schema, e.g., by dividing the cornea into multiple regions and presenting the average, minimal or maximum thickness data, or the ratio of thickness of a microlayer to the total corneal thickness at each region of the cornea. In other examples schema, the bullseye map is presented as a ratio of the thickness of a microlayer in a region of the cornea to the thickness measured of the microlayer in another corneal region. In yet other example schema, the bullseye map is presented as a ratio of the thickness of the microlayer in a specific region of the cornea compared to normative data for that region or for that microlayer. Such mapping schema can also show the progression of thickness or thickness profile of the microlayer from the center to the periphery of the cornea along different meridians of the cornea.

The thickness maps of FIGS. 2A and 2B represent the thickness values in the Bowman's layer for a control sample, e.g., a healthy subject's Bowman's layer. Generally, the thickness values across the Bowman's layer range from 12 microns to 30 microns in thickness; although other thickness ranges may exist for certain subjects and subject populations.

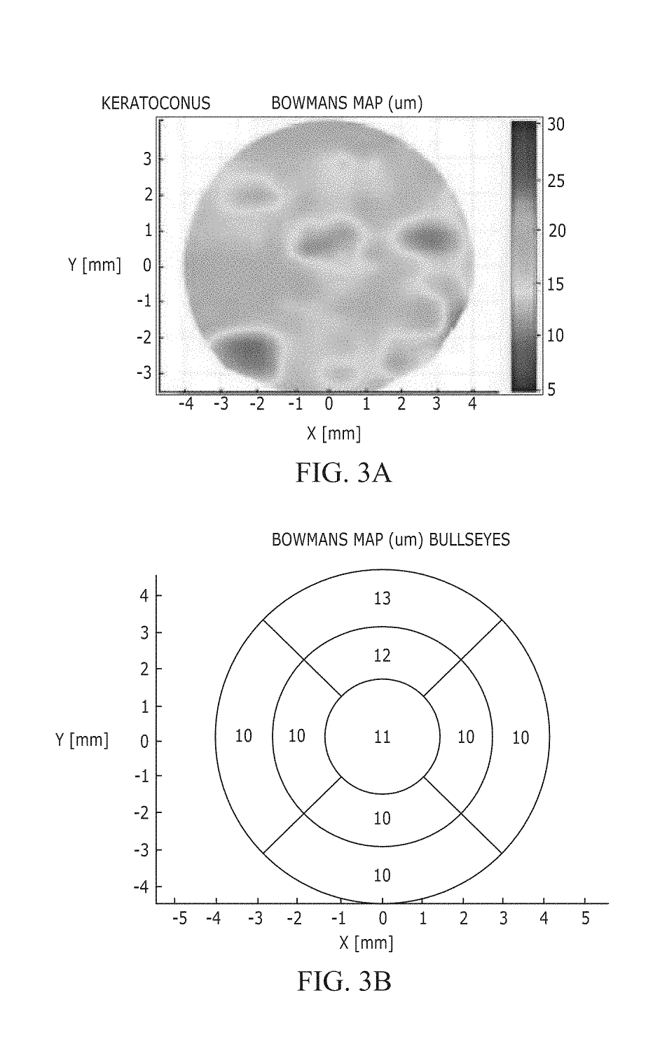

FIGS. 3A and 3B illustrate thickness maps developed by the method 100 from corneal images of a subject that has keratoconus. The keratoconus is identifiable from the thickness mapping of the Bowman's layer, using a number of different diagnostic determinations of the system. The system, for example, may compare the thickness maps of FIG. 3A or 3B to the corresponding thickness maps of FIG. 2A or 2B, and determine thickness difference values across all or certain regions of the Bowman's layer. While pixel-to-pixel comparisons may be performed, generally these comparisons would be region-to-region.

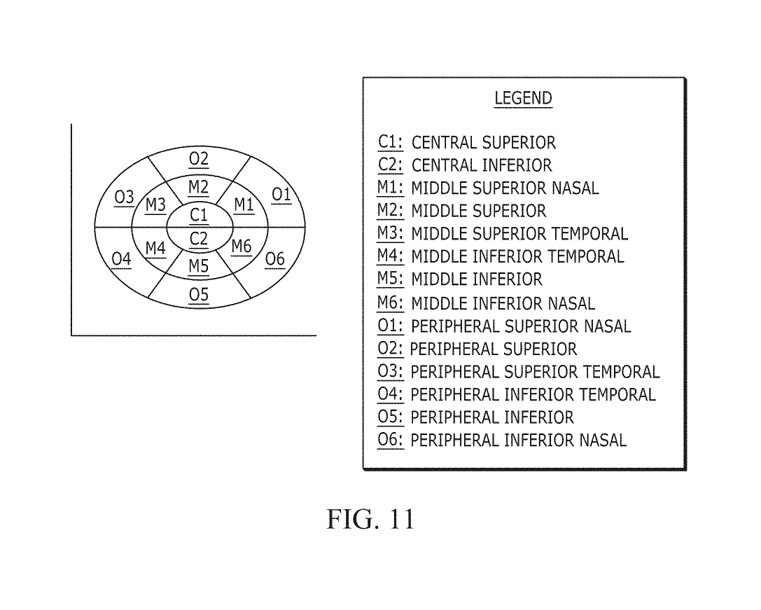

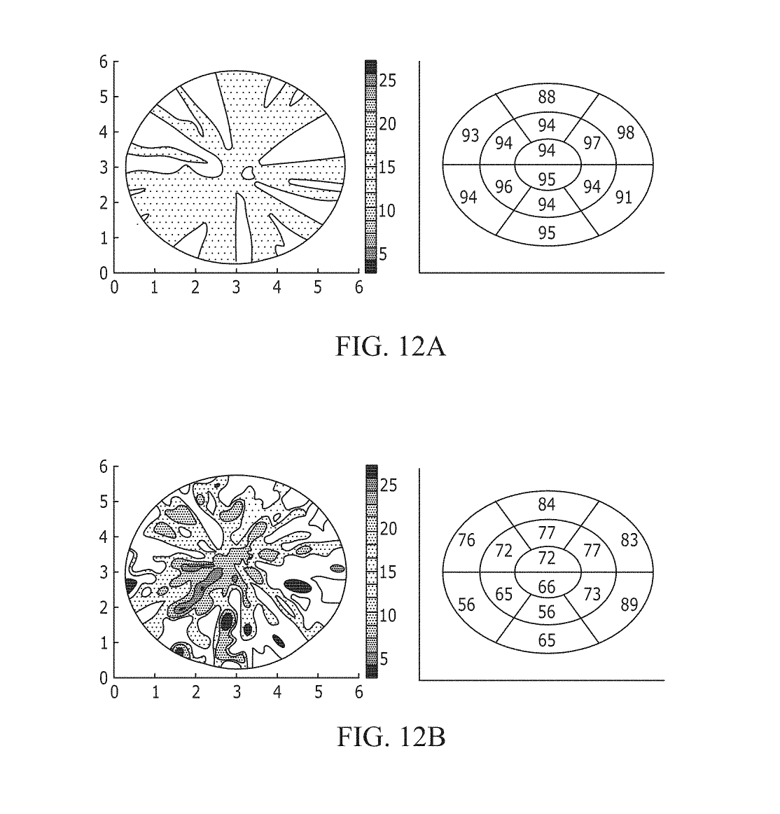

In some examples, the system may determine a composite index value for the Bowman's layer and compare that composite index to a composite index value determined for the control thickness map. For example, indices such as (A) a Bowman's ectasia index (three-dimensional BEI; defined as Bowman's layer (BL) minimum thickness of each region of the inferior half of the cornea divided by BL average thickness of the corresponding region of superior half of the cornea, multiplied by 100) and (B) a BEI-Max (defined as BL minimum thickness of the inferior half of the cornea divided by BL maximum thickness of the superior half of the cornea multiplied by 100) may be used for comparison. An example determination of a three-dimensional BEI is taking the minimum thickness of BL in region C1 divided by the mean thickness of BL region C2, multiplied by 100 (see, e.g., the bullseye thickness map and legend of FIG. 11 and heat map and bullseye example of FIGS. 12A and 12B, respectively). With the present techniques, described herein, such indices are calculated, by the system, using the three-dimensional map of the entire cornea allowing more accurate indexes and index comparisons. The use of three-dimensional BEI demonstrates considerable advantages over conventional techniques. For example, with the present techniques, the thinnest point on the entire cornea (and not just the thinnest point on a 2D scan that goes through a central area of the cornea but might miss the corneal Bowman's thinnest point) may be detected.

In yet other examples, the system compares the thickness maps of FIGS. 3A and 3B against stored threshold thickness values, either overall thickness values of the layer or threshold thickness values of one or more of the regions in the bullseye map. In either case, the amount of difference in thickness, whether between different heat maps or bullseye maps or between thickness maps and threshold data or thickness progression profile, may be further examined using an assurance process that determines if the differences are substantial enough to satisfy a desired assurance level for making a diagnosis. The imaging system may perform an assurance process that not only examines the amount of difference between current corneal images and a control or threshold, but examines particular regions within the Bowman's layer, as thickness differences in certain regions may be more correlative to keratoconus than thickness differences in other examples. Indeed, in some examples, primary regions of interest for diagnosable conditions such as keratoconus may be programmed into the imaging system, regions such as inferior cornea. In other examples, however, the imaging system may be programmed using a learning mode, whether a machine learning algorithm is applied to multiple sets of corneal image data until the machine learning algorithm identifies from the data—the data would include a variety of images for subjects with normal cornea tissue and a variety of images for subjects with keratoconus. From the machine learning, primary regions of interest may be identified, as well as thickness difference values across the different regions. For the latter, for example, the imaging system, may not only determine different threshold thicknesses for different regions in a layer, but the system may determine different high-assurance values for those different regions. For example, assessing the bullseye plot of FIG. 2B against other image data, an imaging system may identify a threshold of 20 microns for each of two opposing radial medial, lateral and inferior regions of the cornea. But as shown in FIG. 3A, only one of the inferior radial regions shows a great correlation to indicating keratoconus. The imaging system, applying the machine learning, can then determine a threshold of 20 microns for each region, but apply a broader assurance band for the left-most region, thereby not flagging a larger number of thickness variations below that threshold, because the region appears less correlative, and thereby less expressive, of keratoconus. The right-most region, however, could be determined to have a very narrow assurance band, meaning that for the same threshold, thickness values below but very close to the threshold would be flagged by the system as indicating, or at least possibly indicating, keratoconus.

The example maps of FIGS. 2A, 2B, 3A, and 3B are determined from thickness maps for the Bowman's layer and used to diagnosis keratoconus, in particular. The same techniques may be used to develop a thickness mapping for any one or more of the corneal microlayers, whichever layers are expressive of the diagnosable condition under examination, including, but not limited to, keratoconus, pellucid marginal degeneration, post-refractive surgery ectasia, corneal transplant rejection and corneal transplant failed grafts, Fuchs' dystrophy, limbal stem cell deficiency and dry eye syndrome. The conditions keratoconus, pellucid marginal degeneration, and post-refractive surgery ectasia are particularly expressed by the Bowman's layer. Therefore, the method 100 may be applied to determine a thickness mapping for that Bowman's layer. Other conditions would result from analyzing the thickness of other microlayers in the cornea. Indeed, the present techniques may be used to determine thicknesses and generate thickness maps for all of these microlayers of the cornea through the same automated process.

In the illustrated example, at operation 108, the imaging system generates a three-dimensional thickness map. The heat map, e.g., FIGS. 2A and 3A, expresses the third dimensional (the XY area being the first two dimensions) thickness data in a color coding or gray scale coding. The bullseye map, e.g., FIGS. 2B and 3B, expresses the third dimension using a numerical thickness score. That numerical thickness score represents an overall thickness value for the region of the bullseye map. That value may be an aggregated thickness value summing the thicknesses over the entire region. That value may be an average thickness value over the entire region, minimal, maximum, variance or standard deviation thickness value over the entire region, or some other thickness value, ratio of the thickness of the region to another region or to a diagnostic index.

Whichever mapping schema is used, the three-dimensional thickness map developed by the system is configured to differentiate normal thickness areas in the heat map (or regions in the bullseye) from thicknesses that express the diagnosable condition. In the illustrated example, the thickness maps further indicate the minimum and maximum thicknesses with the Bowman's layer.

In some examples, multiple different thickness maps may be used to analyze and diagnose the same diagnosable condition. For example, when the condition is dry eye syndrome, a thickness map (or maps) may be generated analyzing the thickness for a plurality of different microlayers that includes the epithelium, the basal epithelial layer, the Bowman's layer, and the Endothelial/Descemet's layers complex of the cornea. In such examples, the three-dimensional thickness map would include combined thicknesses for all these layers summed together. However, depending on the data set and the differences in thicknesses for certain layers, only one of these layers, e.g., the epithelium, may be used. For example, while overall thickness for all these layers combined can indicate dry eye, particular irregularities in the thickness of the epithelium may also indicate dry eye syndrome. That is, different thickness patterns in the epithelium may themselves be an expressive biomarker of dry eye syndrome. For example, the imaging system may assess the thickness map(s) of the corneal epithelium and analyze a central are (or central region of the bullseye) of the cornea which indicates that the dry eye condition results from aqueous deficiency. In another example, the imaging system analyzes the thickness map(s) of the epithelium, in particular a lower or upper area (or region) of the cornea which indicates that lipid deficiency is the cause of the dry eye syndrome.

In some examples, a three-dimensional map (or maps) may be generated by analyzing a thickness of a plurality of different microlayers, which may include two or more of the epithelium (which may include the epithelium without the tear film), the tear film, the basal epithelial layer, the Bowman's layer, or the Endothelial/Descemet's layers complex of the cornea. A three-dimensional thickness map may include, for example, combined thicknesses for all or combinations of these layers summed together. However, depending on the data set and the differences in thicknesses for certain layers, only one of these layers, e.g., the epithelium, may be used. For example, while overall thickness for all or combinations of these layers combined may indicate dry eye, particular irregularities in the thickness of the epithelium may also indicate dry eye syndrome. That is, different thickness patterns in the epithelium may themselves be an expressive biomarker of dry eye syndrome. For example, the imaging system may assess the thickness maps of the corneal epithelium with and analyze a central area (or central region of the bullseye) of the cornea which indicates that the dry eye condition results from aqueous deficiency. In another example, the imaging system analyzes the thickness map(s) of the epithelium, in particular a lower or upper area (or region) of the cornea which indicates that lipid deficiency is the cause of the dry eye syndrome. The imaging system may similarly assess thickness maps with respect to the tear film. In some embodiments, the imaging system may compare thickness maps of the epithelium and the tear film to identify irregularities indicative of dry eye syndrome. As explained in more detail below, three-dimensional maps may also include hyper-reflectivity maps and irregularity maps. Irregularity maps, for example, may include maps illustrating differences in surface topologies of the epithelium, tear film, or other layer from that of an idealized smooth surface. In some embodiments, the imaging system may compare three-dimensional maps of the epithelium or the tear film to identify irregularities indicative of dry eye syndrome.

The imaging system may detect and analyze irregularities through a number of different processes. For example, calculating the standard deviations and variance of the epithelial thickness on each region of a thickness map (e.g., on each region of a bullseye map) will identify irregularities. Such irregularities may be determined for one or more key regions within a thickness map or, in other examples, across the entire thickness map. Which regions and which amounts of irregularities (e.g., the amount of variance) that are analyzed may depend on the underlying condition, with certain conditions associated with certain amounts of irregularities, over certain regions of a thickness map, and for only certain microlayers. As such, the imaging system may be configured to identify for a pre-specified irregularity pattern over a microlayer. While in other examples, the imaging system may analyze the entire cornea for identification of any of a plurality of irregularity patterns, thereafter, identifying to medical professionals which diagnosable conditions have been identified for the subject. Other statistical analyses can be applied to further refine the irregularity pattern identification. Further still, in yet other examples, thickness maps for microlayers may be compared to thickness values of an imaginary regular surface to identify variation patterns.

In various embodiments, the system may generate three-dimensional maps, such as heat maps or bullseye maps for use in diagnosis of dry eye syndrome. For example, image data representative of one or more segmented and measured microlayers may be utilized to measure, identify, and quantify irregularities along the ocular surface, which may include anterior surfaces of the epithelium, tear film, or other layer. In one embodiment, the system may detect pixel differences between the segmented surface of the epithelium and a smooth curve that is created to model the ocular surface to generate a irregularity map, such as within one or more heat or bullseye mapping schemes, that highlights the irregularities of the anterior surface of the ocular surface in isolation of the posterior surface of the layer which could get affected by other conditions.

In some embodiments, three-dimensional maps may be generated for the epithelium, tear film, or other layer. A heat map scheme, for example, may be utilized to depict layer variations, such as variations with respect to one or more of thickness, hyper-reflectivity, or irregularities. In one embodiment, the system may be utilized to detect irregularities along the surface of the epithelium and the tear film. For example, developing the three-dimensional mapping schema may include comparing anterior surfaces of the epithelium and the tear film to a smooth model created to fit the cornea being studied. The difference in pixels between the smooth model and the true surface of the tear film and the epithelium may each be measured and presented to the operator in one or more thickness maps. In one example, a heat map may be coded with different colors based on normative thickness or hyper-reflectivity data of the corneal tear film, epithelium, or other layer. In a further example, a bullseye map may be generated that depict comparisons to normative data over corresponding regions or sections of the epithelium, tear film, or other layer. As introduced above, the imaging system may be configured to detect and analyze irregularities using various processes. For example, the system may calculate standard deviations, variance, or other statistical analytics of epithelial thickness or tear film on various, including each, region of a thickness, irregularity, or hyper-reflectivity map (e.g., on each region of a bullseye map). In some examples, the system may detect pixel differences between the segmented surface of the epithelium and a smooth curve that is created to model the ocular surface to highlight irregularities of the anterior surface of the ocular surface in isolation of the posterior surface of the layer, which could get affected by other conditions.

Other diagnosable conditions include limbal stem cell deficiency, which is diagnosable from the presence of basal epithelial cells thinning or the absence of basal epithelial cells. In such examples, a thickness map of the basal epithelial layer is performed, and the results are diagnosed.

Thus, in some examples, the method 100 may be used to obtain images of a subject using an OCT machine or other imaging device that gives high-resolution cross-sectional images of the cornea. The subject may be instructed to look at different fixation targets representing the different directions of gaze, and the machine will capture images of different segments of the cornea. In other examples, the images may be captured using a wide-angle lens that provides a wide view of the cornea. The machine or other image processor will segment the corneal microlayers, including for example the epithelium, basal epithelial layer, Bowman's layer, and endothelial/Descemet's layers. In one example, the corneal microlayers comprise the epithelium without the tear film layer. For example, segmentation may segment the epithelium from the tear film layer or may segment both the tear film layer and the epithelium layer, wherein one or more layers are subsequently mapped as described herein. One or more of the maps may be further displayed for visual evaluation. In some examples, the segmentation may be presented to the machine operator to allow the operator to review the segmented images and make changes as appropriate. The machine or other image processor will then calculate the thicknesses of the layers from all obtained images, including the epithelium, basal epithelial layer, Bowman's layer, and endothelial/Descemet's layers. The machine or other image processor will stitch the data obtained from the obtained images and combine them to produce a wide color-coded thickness map of the total corneal thickness, epithelium, basal epithelial layer, Bowman's layer, and endothelial/Descemet's layers. The machine or other image processor will create bullseye thickness maps and will compute the diagnostic indices for keratoconus, pellucid marginal degeneration, post-refractive surgery ectasia, corneal transplant rejection and health, Fuchs' dystrophy and dry eye syndrome. As noted above, in some system embodiments, the machine or image processor may calculate thickness from less than all of the obtained images, such as only those desired or relevant to a condition. For example, the machine or image processor may segment from the images one or more of total cornea, epithelium, with or without the tear film, basal epithelial layer, tear film, Bowman's layer, or endothelial/Descemet's layers. The machine or image processor may further or calculate thickness for one or more of total cornea, epithelium, with or without tear film, the tear film layer, basal epithelial layer Bowman's layer, or endothelial/Descemet's layers. In some examples where the diagnosable condition is Fuchs' dystrophy and/or corneal graft, the machine or other image processor may produce a color-coded three-dimensional map of the entire Endothelium/Descemet's layer of the cornea. Relative thickening of the Endothelium/Descemet's layer and thickening and irregularity compared to a normal value will be highlighted on a color-coded three-dimensional map. A separate bullseye map may be developed and will show the average thickness of the Endothelium/Descemet's layer in different parts of the cornea which is diagnostic for the condition. Progression or stability of the condition may be detected by comparison of serial maps and thickness data obtained from follow up maps.

In some examples where the diagnosable condition is keratoconus, pellucid marginal degeneration, and/or post-refractive surgery ectasia, the machine or other image processing machine will produce a color-coded three-dimensional map of the entire Bowman's layer. Relative thinning of the Bowman's layer and thinning compared to a normal value will be highlighted on the color-coded map. A separate bullseye map will show the average and minimum thickness of the Bowman's layer in different parts of the cornea which are diagnostic for the condition. Progression or stability of the condition will be detected by comparison of serial maps and thickness data obtained from follow up maps.

In some examples where the diagnosable condition is dry eye patients, the machine or other image processor will create a color-coded three-dimensional map of the entire cornea and calculate the irregularities of the epithelium of the cornea. Relative irregularity compared to a normal value will be highlighted on the color-coded map. A separate bullseye map will show the average thickness and the variation of the layer thickness in different parts of the cornea which is diagnostic for the condition. The machine or other image processor identifying more irregularities in the central part of the cornea thereby diagnosing aqueous deficiency, which diagnosis may be displayed to the operator, while more irregularities on the lower or upper part of the cornea are diagnosed by the machine or other image processor as lipid deficiency dry eye syndrome or Meibomian gland dysfunction, which may be displayed to the operator. Progression or stability of the condition will be detected by comparison of serial maps and thickness data obtained from follow up maps.

In some examples where the diagnosable condition is limbal stem cell deficiency, the machine or other image processor will generate a color-coded three-dimensional map of the entire the basal epithelial layer and then determine relative thinning or absence of the layer basal epithelial, which is diagnostic of limbal stem cell deficiency. If the condition is identified by the machine or other image processor, that diagnosis is displayed to the operator.

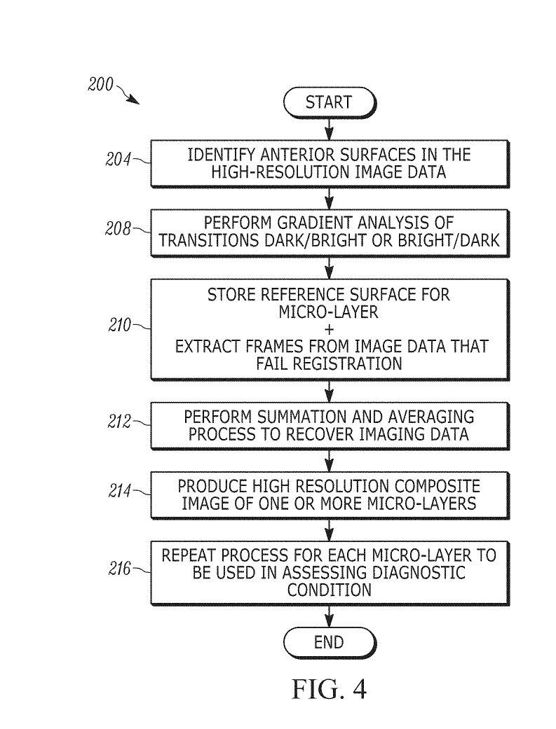

FIG. 4 illustrates a computer-implemented segmentation process 200 as may be implemented by operations 104 in FIG. 1, in accordance with an embodiment. In the illustrated example, the high-resolution images are segmented to identify image data for one or more of the biologically-defined microlayers. Initially, an optional image registration process is performed on the high-resolution images, in particular by identifying a series of surface layers that correspond to layers at which an image transitions from one microlayer of the cornea to another microlayer. The registration process may include, at an operation 204, identifying an anterior surface of one of the microlayers in the cornea. This anterior surface may be of any of the epithelium, basal epithelial layer, Bowman's layer, or endothelial/Descemet's layers complex, for example. In some embodiments, the epithelium may be the epithelium layer without the tear film. In one embodiment, the anterior surface may be the tear film, such as an anterior surface thereof. In other examples, anterior and posterior surfaces of the microlayers may be identified.

The anterior surface can be identified using a contrast identification-based algorithm, for example, an algorithm identifying gradient changes from dark to bright or bright to dark, in an image. In an example, gradient method and graph theory techniques were adapted to the cornea and used to segment the corneal layers. Furthermore, in some examples, particular image filters are combined with the image analysis to more accurately identify transitions.

With continued reference to FIG. 4, while not shown, operation 204 may be followed by an averaging operation applied to the high-resolution images for reducing noise and improving image quality.

At an operation 208, a gradient analysis is performed on the received high-resolution images. The gradient analysis identifies gradient changes of a threshold amount, whether the gradient change is dark to bright or bright to dark, for example using a graph theory algorithm. In general, at operation 208, an automatic segmentation of the corneal microlayers is achieved by detecting the interfaces between one layer from another layer. From the gradient changes, the anterior surface is identified and stored as the registered reference surface, at operation 210. In other examples, as described, the reference surface may be determined from analyzing an anterior surface and a posterior surface. The operation 210 may also perform alignment of subsequent images to this reference surface. That alignment may be done electronically through image processing instructions. The alignment may include side to side and/or rotational alignment. If the anterior surface in one or more of the frames does not fit other frames registered surface secondary to a course movement of the patient, that frame is extracted and excluded. This frame extraction is provided for each image that does not satisfy a registration condition.

Using the reference surface, the system may be programmed to select from the programmed alignment algorithms and apply the one or more algorithms to achieve a suitable registration and, in some examples, to achieve the best registration.

Every subsequent high-resolution image may be compared to the registered reference, and, after the operation 210 extracts those frames that do not satisfy the registration condition, at operation 212, images may be averaged over a certain cycle, e.g., after 25 frames, 50 frames, 100 frames, or more frames or less. That is, at operation 212, the process 200 applies to the remaining frames a summation and averaging process to produce, at operation 214, a segmented high-resolution composite image of the one of the biologically-defined microlayers. The process 200 may repeat for each of the microlayers in the cornea, via operation 216. For example, the operation 226 may repeat the process 200 identifying a plurality of contrast transition surfaces, where the transition surfaces correspond to interfaces of the microlayers in the cornea. The process 200 may be repeated for microlayers adjacent to any preceding surface, and this process may repeat until each biologically-defined microlayer is mapped out.

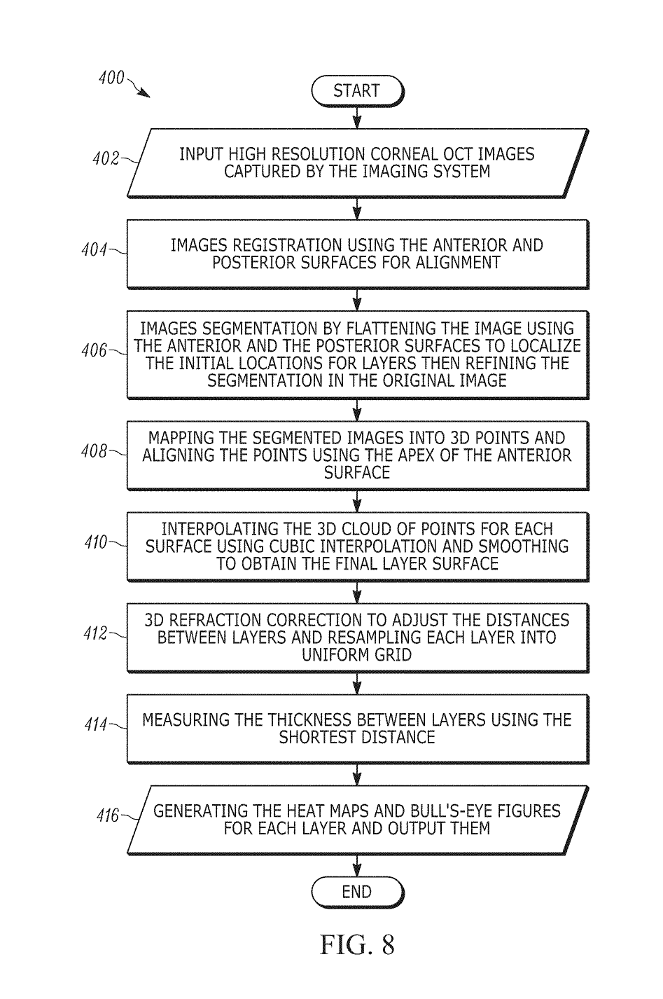

In the illustrated example, segmentation occurs without initial registration, and instead, after the segmentation (e.g., microlayer extraction of operation 210) applied to each image, the images may then be summed and averaged to produce the segmented high-resolution composite image. Other example embodiments of the present techniques are provided in reference to FIGS. 8-10.

Image preprocessing may be performed to enhance optical coherence tomography (OCT) images, which may facilitate automatic segmentation of corneal microlayers and thickness data extraction, e.g., the epithelium with or without the tear film, tear film, basal epithelial layer, Bowman's layer, or endothelial/Descemet's layers.

For example, in one embodiment, image preprocessing is done in order to enhance the optical coherence tomography (OCT) images in order to facilitate automatic segmentation of corneal microlayers and thickness data extraction, namely, the epithelium, basal epithelial layer, Bowman's layer, and endothelial/Descemet's layers. Preprocessing of the OCT images may include registration and averaging of the images to reduce noise to signal ratio and correct for patients' movements artifacts. The registered frames and averaged images produce a final averaged frame comprising a composite image.

In some embodiments, preprocessing of the OCT images may include registration and averaging of the images to reduce noise to signal ratio and correct for patients' movements artifacts. Such preprocessing may be performed using a segmentation-based registration and averaging, as described herein. In one embodiment, preprocessing may include processing frames row-wise and the two processed results are combined to produce a binary template. The two frames may be aligned vertically, for example by cross-correlating vertical projections of binary templates. The two frames may then be aligned horizontally by horizontal 2D correlation of the binary templates. The registered frames may then be averaged to produce the final averaged frame comprising a composite image.

In one embodiment, pre-processing similar to that described with respect to FIG. 4 may be utilized to improve signal to noise ratio via a segmentation-based registration and averaging process. For example, corneal images may be registered and averaged according to a process including removing artifacts from the raw images; segmenting the corneal epithelium and endothelial boundaries to use them to register the images; registering frames with a selected reference frame based on the epithelium and endothelial boundaries; and aligning the registered frames and averaging to produce a final average frame.

Removing artifacts from the raw images may include removing a top horizontal artifact using a vertical projection of each frame. In one example, the frame may be pre-processed column-wise and row-wise wherein pixels that are below a certain threshold are set to zero. The frame may be median filtering and post-processing the frame to remove noise.

Segmenting the corneal epithelium and endothelial boundaries to use them to register the images may include segmentation, which may be automatic segmentation by an imaging processor, of the corneal epithelium and endothelial boundaries to use them to register the images. The epithelial boundary may be estimated by extracting the top points of the frame and then using Random sample consensus (RANSAC) method to fit those points to a second order polynomial, for example. Similarly, the corneal endothelium may be estimated by extracting the bottom points of the pre-processed frame and then using RANSAC method to fit them to a second order polynomial, for example.

Registering frames with a selected reference frame based on the epithelium and endothelial boundaries may include selection of a random reference frame from the captured raw frames. Each frame may be registered with the reference frame based on the segmentation of the corneal epithelium and endothelium layer boundaries. The correspondence between the two layers in the frames may be determined by using the vertex of each estimated layer boundary. A geometric transformation may then be estimated to transform the frame to be registered to be aligned with the reference frame. The registered frames may then be aligned and averaged to produce the final averaged frame, e.g., a composite image. Notably, in one embodiment, the epithelial boundary as described in the above example may alternatively be an anterior boundary corresponding to a boundary of any microlayer and the endothelial boundary may be a posterior boundary corresponding to a boundary of any microlayer.

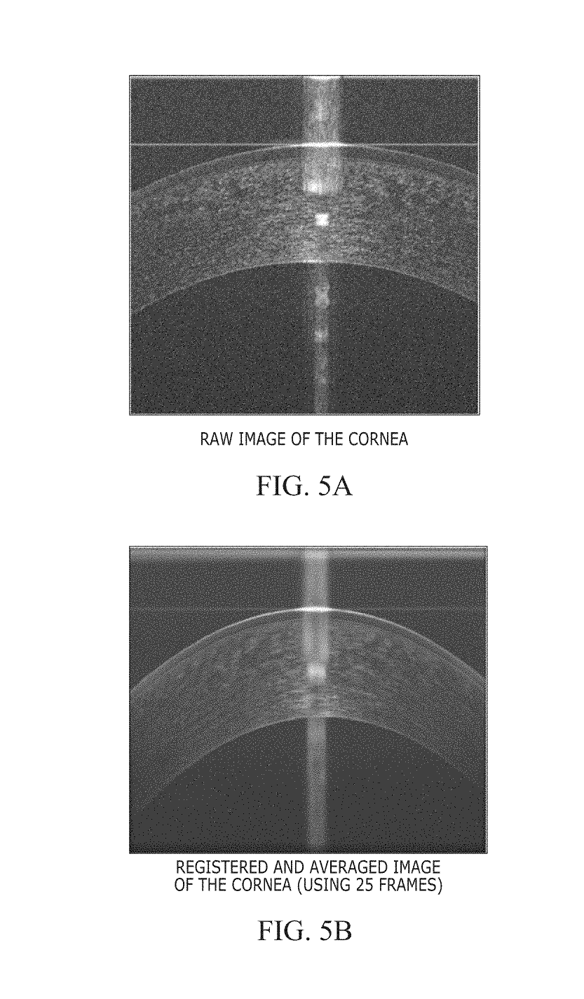

FIG. 5A illustrates a first raw high-resolution image of a cornea. FIG. 5B illustrates registered and averaged images of the cornea, using 25 frames, comprising a composite image. From the comparing the two images, FIG. 5B illustrates the high contrast image with the great certainty to visualize the corneal microlayers.

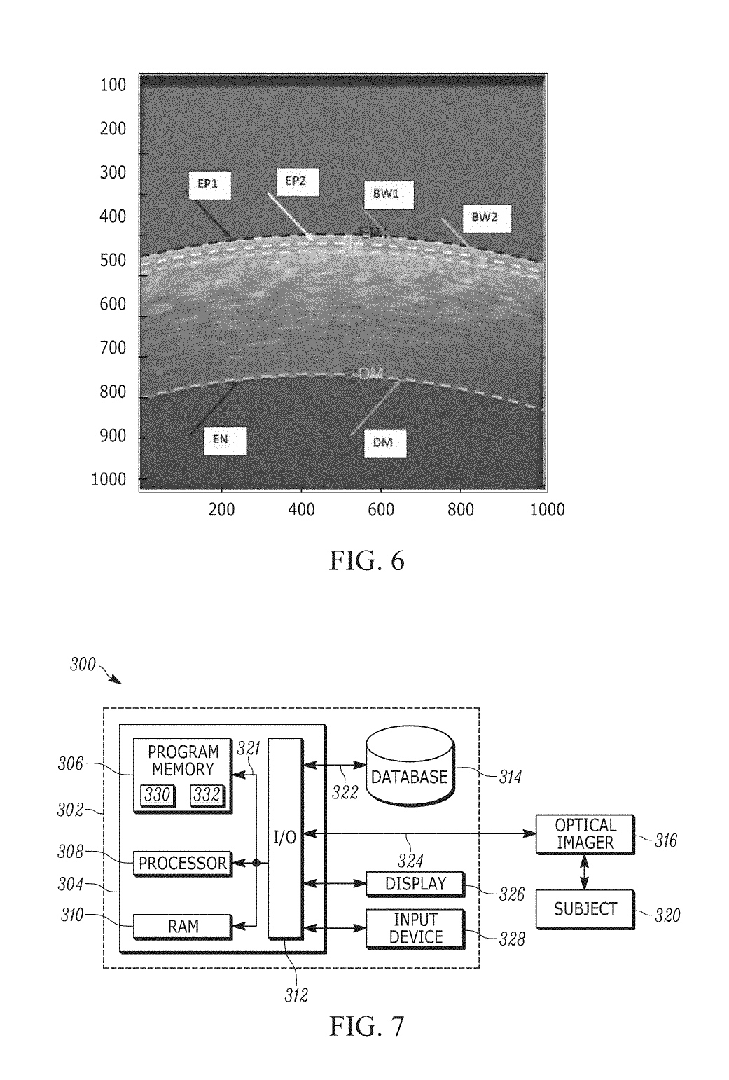

FIG. 6 illustrates an entire cornea with microlayers mapped out and demarcated by their respective anterior and posterior surfaces for each layer, in accordance with the process of FIG. 4. For the cornea Layers, the epithelium is the layer from EP1 to EP2, the basal epithelial layer is the layer from EP2 to BW1, the Bowman's layer is the layer from BW1 to BW2. The Endothelial/Descemet's layer is the layer from DM to EN.

Generally speaking, the process 200 may be used to identify a transition to an anterior interface of the epithelium, an epithelium/basal epithelial layer interface, a basal epithelium/Bowman's interface, Bowman's/stroma interface, an anterior interface of the endothelial/Descemet's layers, an interface of the endothelial/Descemet's layers, and an aqueous humor. In one example, the process 200 may be used to identify a transition to an anterior interface of the epithelium with respect to the tear film layer or the anterior surface of the tear film layer.

FIG. 7 illustrates an imaging system 300 illustrating various components used in implementing any of the techniques described herein. An image processing device 302 is coupled to a corneal optical imager 316 that collects high-resolution corneal images for a subject 320. The optical imager 316 may be any optical imaging system such as an OCT imager communicatively coupled to an image processing device 302, which may be a dedicated imaging system for example. In some examples, the imaging system 300 may be partly or wholly implemented on an optical imaging system, such as an OCT imager.

The optical imager 316 collects and stores corneal image data on the subject 120, as raw data, processed data, or pre-processed data.

In some examples, the system 300 is operable in a first mode, called a training mode, where the system 300 collects data and develops data on healthy corneal tissue.

In a second mode, called the analysis mode, the system 300 collects subsequent corneal tissue images and compares analyzed image data against the image data of healthy subjects captured in the training mode. Both the training mode data and the analysis mode data include generating the three-dimensional thickness mapping data described herein.

In a healthy subject, training data may include data from a number of subjects compiled together as aggregated training data. In some examples, that aggregated training data is coded with demographic data, such that the system 300 may use demographic-specific subsets of that aggregated data when develop training models for a subject associated with a particular demographic group.

The optical imager 316 is communicatively connected to the image processing device 302 through a wired or wireless link 324. For the former, the optical imager 316 may capture and store corneal images, and a user or care provider may connect the optical imager 316 to the image processing device 302 through a Universal Serial Bus (USB), IEEE 1394 (Firewire), Ethernet, or other wired communication protocol device. The wireless connection can be through any suitable wireless communication protocol, such as, WiFi, NFC, iBeacon, etc.

The image processing device 302 may have a controller 304 operatively connected to a database 314 via a link 322 connected to an input/output (I/O) circuit 312. It should be noted that, while not shown, additional databases may be linked to the controller 304 in a known manner. The controller 304 includes a program memory 306, the processor 308 (may be called a microcontroller or a microprocessor), a random-access memory (RAM) 310, and the input/output (I/O) circuit 312, all of which are interconnected via an address/data bus 321. It should be appreciated that although only one microprocessor 308 is shown, the controller 304 may include multiple microprocessors 308. Similarly, the memory of the controller 304 may include multiple RAMS 310 and multiple program memories 306. Although the I/O circuit 312 is shown as a single block, it should be appreciated that the I/O circuit 312 may include a number of different types of I/O circuits. The RAM(s) 310 and the program memories 306 may be implemented as semiconductor memories, magnetically readable memories, and/or optically readable memories, for example. The link 324 operatively connects the controller 304 to the capture device 316, through the I/O circuit 312.

The program memory 306 and/or the RAM 310 may store various applications (e.g., machine readable instructions) for execution by the microprocessor 308. For example, an operating system 330 may generally control the operation of the image processing device 302 and provide a user interface to the device 302 to implement the processes described herein. The program memory 306 and/or the RAM 310 may also store a variety of subroutines 332 for accessing specific functions of the image processing device 302. By way of example, and without limitation, the subroutines 332 may include, among other things: obtaining, from an optical imaging system, a plurality of high-resolution images of a cornea of the eye; segmenting, using an image processor, a plurality of high-resolution images of a cornea of the eye, to identify one or more of the plurality of biologically-defined microlayers of the cornea, the plurality of high-resolution images comprising a plurality of images for a plurality of biologically-defined microlayers of the cornea; determining thickness data for each of the identified one or more of the plurality of biologically-defined microlayers, from the segmentation of the plurality of high-resolution images; developing, from the thickness data for each of the identified one or more of the plurality of biologically-defined microlayers, a thickness map, the thickness map identifying differences in corneal thickness across the identified biologically-defined microlayer, wherein the thickness map is correlated to a diagnosable condition of the cornea; and displaying the thickness map to provide an indication of the diagnosable condition. In other examples, the subroutines 332 may include instructions to: segment, using an image processor, a plurality of high-resolution images of a cornea of the eye, to identify one or more of the plurality of biologically-defined microlayers of the cornea, the plurality of high-resolution images comprising a plurality of images for a plurality of biologically-defined microlayers of the cornea; determine thickness data for each of the identified one or more of the plurality of biologically-defined microlayers, from the segmentation of the plurality of high-resolution images; develop, from the thickness data for each of the identified one or more of the plurality of biologically-defined microlayers, a thickness map, the thickness map identifying differences in corneal thickness across the identified biologically-defined microlayer, wherein the thickness map is correlated to a diagnosable condition of the cornea; and display the thickness map to provide an indication of the diagnosable condition. In other examples, the subroutines 332 may include instructions to: perform a two-surface registration on each of a plurality of high-resolution images of the cornea, the plurality of high-resolution images comprising a plurality of images for a plurality of biologically-defined microlayers of the cornea, and generate a high-resolution composite image of the cornea, wherein the two-surface registration comprises an anterior surface registration and a posterior surface registration; segment the high-resolution composite image to identify each of the plurality of biologically-defined microlayers of the cornea, wherein segmentation of the high-resolution composite image comprises flattening the high-resolution composite image and performing a vertical projection of a flattened rendition of the high-resolution composite image to produce a segmented high-resolution composite image; determine the thickness of at least one of the plurality of biologically-defined microlayers of the cornea from the segmented high-resolution composite image; develop a thickness map for at least one of the plurality of biologically-defined microlayers of the cornea, the thickness map identifying visual differences in thickness across the at least one of the plurality of biologically-defined microlayers, wherein the thickness map is correlated to a diagnosable condition of the cornea; and display the thickness map to provide a visual indication of the diagnosable condition. In other examples, the subroutines 332 may include instructions to: generate a high-resolution composite image of the cornea from a plurality of high-resolution images of the cornea using a multiple surface registration on the plurality of high-resolution images of the cornea, the plurality of high-resolution images comprising a plurality of images for a plurality of biologically-defined microlayers of the cornea, the plurality of high-resolution images of the cornea each being curved images with an apex; segment the high-resolution composite image to identify each of the plurality of biologically-defined microlayers of the cornea using a multiple surface flattening on the high-resolution composite image, the segmentation generating a segmented high-resolution composite image; determine the thickness of at least one of the plurality of biologically-defined microlayers of the cornea from the segmented high-resolution composite image; develop a thickness map for the at least one of the plurality of biologically-defined microlayers of the cornea, the thickness map identifying visual differences in thickness across the at least one of the plurality of biologically-defined microlayers; and display the thickness map. The subroutines 332 may include subroutines to executed any of the operations described herein, including, for example, those of FIGS. 1, 4, and 8-10.

The subroutines 332 may include other subroutines, for example, implementing software keyboard functionality, interfacing with other hardware in the device 302, etc. The program memory 306 and/or the RAM 310 may further store data related to the configuration and/or operation of the image processing device 302, and/or related to the operation of one or more subroutines 332. For example, the data may be data gathered from the system 316, data determined and/or calculated by the processor 308, etc.

In addition to the controller 304, the image processing device 302 may include other hardware resources. The device 302 may also include various types of input/output hardware such as a visual display 326 and input device(s) 328 (e.g., keypad, keyboard, etc.). In an embodiment, the display 326 is touch-sensitive, and may cooperate with a software keyboard routine as one of the software routines 332 to accept user input. It may be advantageous for the image processing device to communicate with a broader network (not shown) through any of a number of known networking devices and techniques (e.g., through a computer network such as an Intranet, the Internet, etc.). For example, the device may be connected to a database of corneal image data, a database of healthy corneal image data, and a database of corneal image data for subjects experiencing one or more diagnosable conditions such as those listed herein above.

FIGS. 8-10 illustrate further computer-implemented processes for evaluating the eye of a subject, in accordance with some embodiments. As with the process 100, the process 400 may be implemented wholly or partly on an optical imaging system, such as an OCT machine, or on any suitable image processor (e.g., imaging system).

In the illustrated embodiment of the process 400, high-resolution OCT images are captured at an operation 402, and an image registration is performed on the high-resolution OCT images using anterior and posterior corneal surfaces and corneal apex for alignment at an operation 404. The registration may occur between a captured reference image and subsequent captures images. For example, multiple images of the cornea may be captured for all corneal regions. Those images may be radial or raster cut images, for example. In some examples, several images of the exact same region of cornea will be captured. These captured images are registered at the operation 404. The operation 404 may register images captured for each of the regions of the cornea in this way.

Image segmentation is then performed at an operation 406. In the illustrated example, the image segmentation is performed by double flattening the image and producing an averaged image for the cornea (or, in other examples, an averaged image for reach region of the cornea) using the anterior and posterior surfaces to localize initial conditions for layers, and from there a refining segmentation of the original image is performed. In some examples, the segmentation operation is performed without the registration and/or without the averaging operations of 404. That is, operation 404 is optional. In such examples, the captured high-resolution images of operation 402 may be obtained directly by the operation 406, after capture by the optical imaging device, where segmentation and thickness mapping operations are then performed. In some embodiments, the segmentation operation and further operations of process 400 are performed on one or more of the received high-resolution images from the optical imaging system.

In the illustrated example, at an operation 408, the segmented, averaged images for each corneal region are analyzed, thickness data is obtained, that data for reach corneal region is mapped into three-dimensional points and alignment of some or all of the points is performed using the apex of the anterior surface for alignment. As discussed above, in some embodiments using image registration, thickness maps may be formed through operations that register images using the anterior and posterior surfaces as well as the apex. By registering images using the two surfaces, in these examples, rotational motion artifacts may be accounted for, and images may be more accurately registered without flattening the image to preserve the apex of the cornea and use it as a reference and thus compensate for lateral motion artifacts. Additionally, by aligning images using the corneal apex, which represent the center of the cornea, motion artifacts (e.g., resulting from patient moving their eyes during capturing of the images) may be corrected.

At an operation 410, interpolation is performed on the three-dimensional points from the operation 408, and that interpolation is performed for each surface using, in this example, cubic interpolation and smoothing to obtain a final layer surface. At an operation 412, three-dimensional refraction correction algorithm is applied on each corneal microlayer to correct for optical distortions resulting from light refraction at different corneal interfaces with different refractive indices.

Additionally, resampling of each layer into a uniform grid may be performed at the operation 412. At operation 414, the thickness of one or more microlayers is determined by measuring the short distance between microlayers as the thickness. At an operation 416, the machine generates three-dimensional thickness heat maps and a bullseye display for each microlayer and displays them to an operator. For example, an operator may select which microlayer the machine is to display, and the machine displays the corresponding three-dimensional thickness heat map and bullseye display. In another aspect, the machine may provide a list of selectable analyses from which the machine may use to develop a three-dimensional mapping schema as described herein. For example, a user may select from a plurality of diagnosable conditions from which thickness maps may be generated that provide correlatable analysis of the image data with that of a selected diagnosable condition, which may include generating thickness maps that visually identify differences in thickness of corneal microlayers across the thickness map or provide a visual indication of the diagnosable condition. Thickness maps may include heat or bullseye maps. The machine may include one or more selectable statistical parameters with respect to thickness from which three-dimensional mapping schema may be developed. Such parameters may include comparisons with normative data, previously obtained image data of the cornea or its microlayers, or models, such as smooth models, for example. Parameters may also include indices. Any of the above parameters or other parameters may be correlated to diagnosis of a diagnosable condition.

The processes herein, such as process 400, include a number of advantages that improve computer operation involved with cornea image processing and diagnostics. The registration operation 404, for example, may provide better accuracy due to the use of dedicated anterior surface and posterior surfaces on the collected images. The process is further enhanced, and motion artifacts are corrected by the addition, in this example, of matching the anterior and posterior surfaces between frames. The segmentation operation 406 improves computer operation, as well, by introducing robust processing and artifact removal for corneal OCT images, through flattening the image using both the anterior and posterior surfaces, and uniquely identifying other layers from the vertical projections of the two flattened images. The segmentation also allows specific refinements such as, for vertical projection, periphery parts of the image may be excluded, and the central part of the image may be excluded, but only if a central artifact is detected. Such refinements can produce enhanced peak detection, for example. Operation 414 provides further advantage, including defining the thickness as the shortest distance between each two successive microlayers, instead of the distance measured across the normal to a surface, which can be inaccurate.

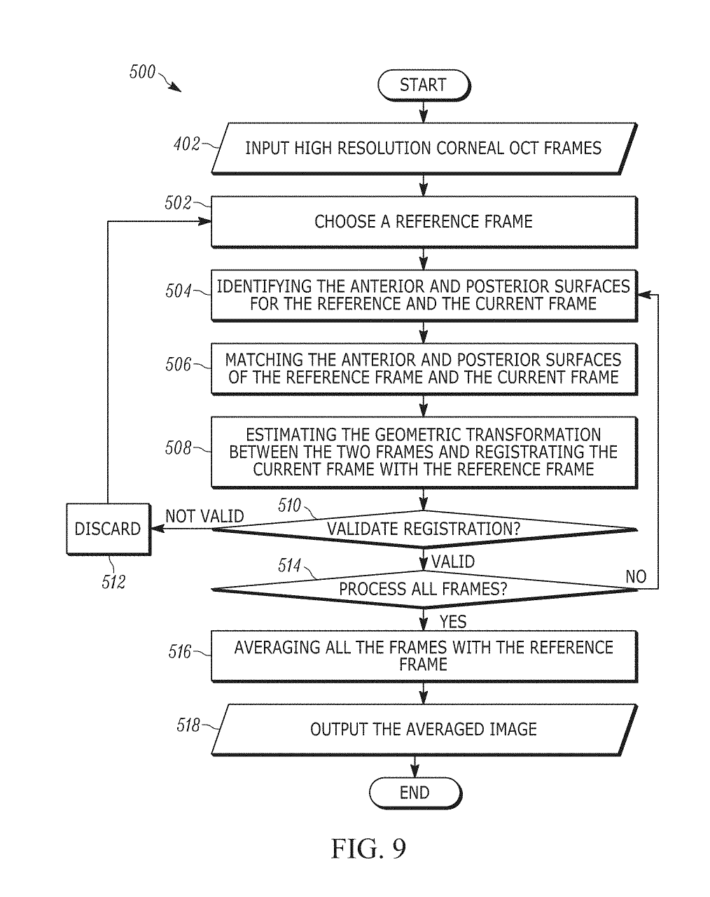

FIG. 9 illustrates an example image registration process 500 that may be performed by the operation 404 of FIG. 8. The high-resolution OCT images are obtained at the operation 402. At operation 502 a reference frame is chosen by the machine, such as the first received image, or the first received image with a determined image quality, such as with a signal-to-noise ratio above a threshold. At an operation 504, the anterior and posterior corneal surfaces of the reference image and for the current frame being compared to the reference image are determined. The anterior and posterior surfaces of both are matched to one another at an operation 506. From the matching, a geometric transformation between the two frames is estimated at an operation 508 and registration is performed based on the geometric transformation.

An operation 510 determines if the registration is valid. For example, the operation 510 may perform an automated image processing edge blur analysis and/or image contrast analysis. In some examples, an operator may subjectively assess image quality and clarity of displayed microlayers.

If the registration is not valid, the registration is discarded at an operation 512 and the process repeats by choosing a new reference frame at operation 502. In other examples, the process may discard the registration and return to operation 504 to perform another attempted transformation and registration. In such examples, the process may return to operation 504 a given number of times, such as twice, before returning to operation 502 to determine a new reference frame. If instead, the registration is valid at operation 510, then the process (operation 514) determines if there are any more frames to process, and either returns to operation 504 or then performs an averaging of all processed frames with the reference frame at an operation 516, from which an averaged image is output at operation 518, and that averaged image is used for segmentation at operation 406.

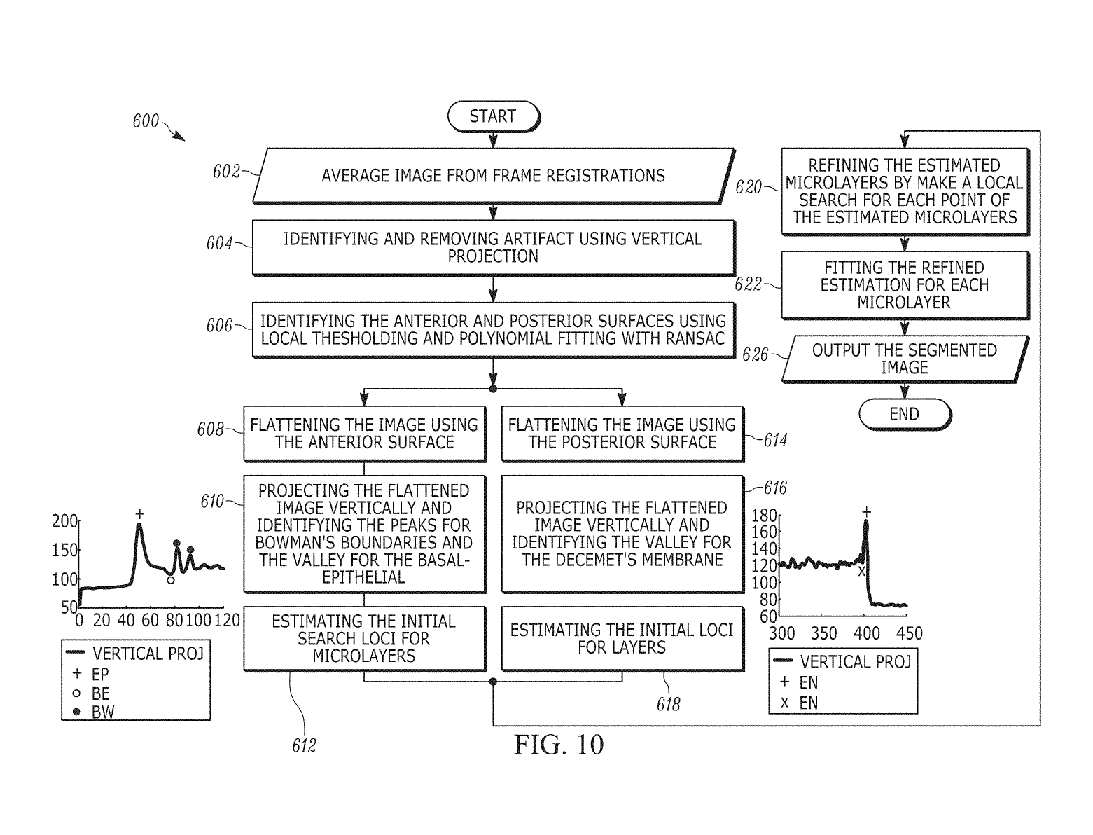

FIG. 10 illustrates an example process 600 that may be implemented as the segmentation operation 406 of FIG. 8. In the illustrated embodiment, the averaged image from the registration operation 404 is provided at operation 602. The registration and averaging processes, however, are optional and may not be performed. For example, in some embodiments, the captured high-resolution images of operation 402 are passed to the operation 604 (of operation 406) after capture by the optical imaging device, bypassing the registration and averaging processes of operation 404. In any event, in the illustrated embodiment, artifact identification and removal using vertical projection is performed on the averaged image at an operation 604. Anterior and posterior surfaces are identified using local thresholding and polynomial fitting from a random sample consensus (RANSAC) iterative process at an operation 606. That operation 606 leads to different operation pipes, one on each of the anterior surface and the posterior surface. For example, the image data from operation 606 may be buffered into two different identical copies each of which is analyzed on each of the two double flattening pipes illustrated. These copies may be exact duplicates of the entire corneal image, while in other examples, truncated versions of the entire corneal image may be used, an anterior rendition and a posterior rendition, respectively.

A flattening of the averaged image is performed using the anterior surface at an operation 608. Next, the flattened image is projected vertically and the peaks for the Bowman's layer boundaries are identified and the valley for the basal-epithelial layer is identified at an operation 610. An operation 612 then estimates the initial search loci for these microlayers. Any number of microlayers may be identified at the operation 610, including, for example, the epithelium, the basal epithelium, the Bowman's layer, and the endothelium/Descemet's layer, by way of example. In various embodiments, the epithelium may be the epithelium layer without the tear film layer. The tear film may also be an identified microlayer at operation 610.

In a separate operation, flattening of the averaged image (e.g., a copy thereof), is performed using the posterior surface by operation 614. Next, the flattened image is projected vertically to identify the valley for the Descemet's layer at an operation 616, after which the loci of the microlayers (e.g., endothelium/Descemet's complex layer) are estimated at a block 618.

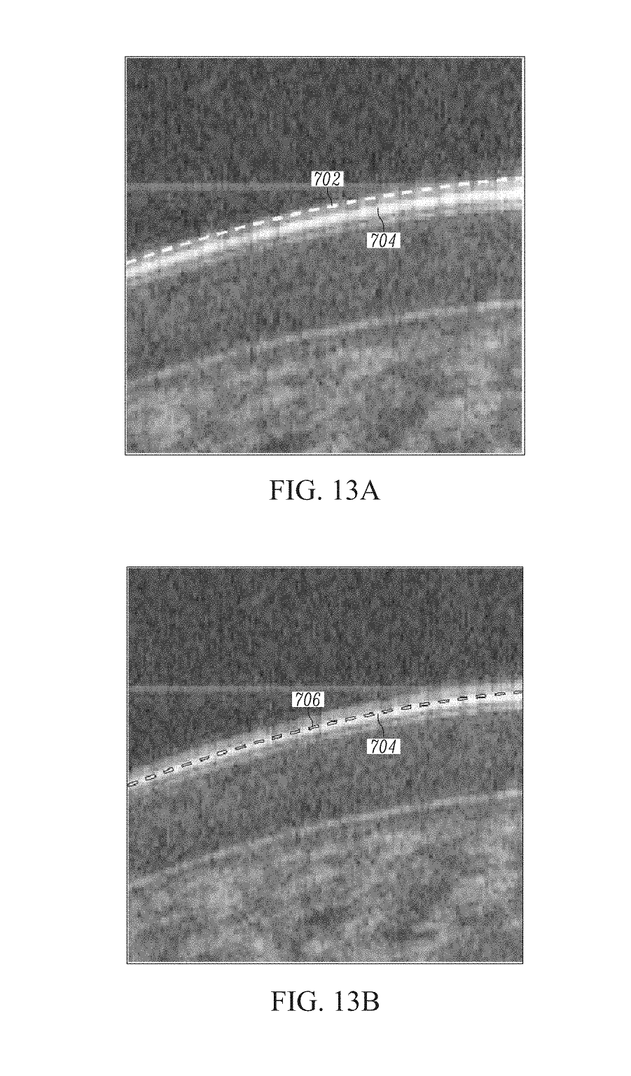

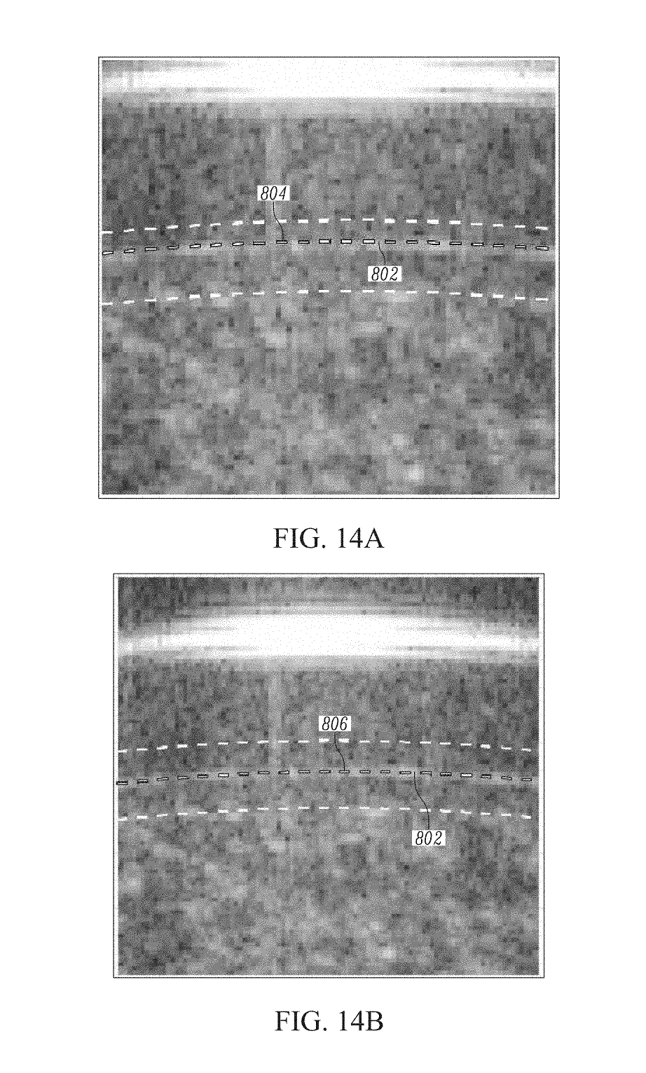

The estimated loci of microlayers output from operations 612 and 618 (see example estimated loci plots in legends) are then combined, and an operation 620 refines the estimate microlayers by making a local search for each point of the estimated microlayers, e.g., the local peaks in the loci plots. At an operation 622, a fitting is performed on the refined estimation of each microlayer identified. FIGS. 13A and 13B illustrate an example segmentation refinement for an anterior boundary of the epithelial layer, as performed by the operations 620 and 622. As shown in FIG. 13A, a high-resolution OCT image (e.g., an averaged image received at operation 602 from the frame registration process of FIG. 9) has been segmented using a double flattening process. A segmentation line 702, shown as a dashed line, and an anterior boundary of the epithelial layer 704 are illustrated. This initial segmentation line 702, while close, is slightly off-centered from the epithelial layer 704. In FIG. 13B, however, the same segmented OCT image is shown after the refining automatic segmentation operation 620. The result is a refined segmentation line 706 that more closely matches the actual anterior boundary of the epithelial layer 704. FIGS. 14A and 14B illustrate another example. A segmented OCT image is shown of an anterior boundary 802 of a Bowman's layer. An initial segmentation line 804 is shown in FIG. 14A, while a refined segmentation line 806, after the operations 620, is shown in FIG. 14B. In some embodiments, a refinement process is performed on each of the images, and where the initial segmentation was accurate, the refinement will not result in changes to the segmentation.

In an example, the operation 620 refines the microlayers segmentation by making a local search for each point of the estimated microlayers, e.g., the local peaks in the loci plots forming the segmentation line. The initial segmented image from the double flattening process, e.g., combining the two pipeline images, is analyzed by the system to search locally for the best alternative for each point in the estimated initial guess of the microlayer boundaries (e.g., the initial guess of the segmentation lines in FIGS. 13A and 14A). These microlayer boundary estimates are processed in order to ensure that there is no overlapping or crossing between layers and each microlayer search window is limited by its predecessor and successor microlayers. In an example, the initial segmentation line is filtered using a proposed filter given by

$$\frac{1}{16} * [ \begin{matrix}1 & 1 & 1 \\ 1 & 8 & 1 \\ 1 & 1 & 1\end{matrix} ]$$

This filter takes the mean of the 8-neighbors of the center pixel of a segmentation line and then averages it with the center pixel to give more emphasis on the center pixel of that segmentation line. The result is

$$\frac{1}{16} * [ \begin{matrix}1 & 1 & 1 \\ 1 & 8 & 1 \\ 1 & 1 & 1\end{matrix} ] = \frac{1}{2} ( \frac{1}{8} * [ \begin{matrix}1 & 1 & 1 \\ 1 & 0 & 1 \\ 1 & 1 & 1\end{matrix} ] + [ \begin{matrix}0 & 0 & 0 \\ 0 & 1 & 0 \\ 0 & 0 & 0\end{matrix} ] )$$

To reduce the effect of speckle noise or background noise, the refined segmentation lines are smoothed with a median and moving average filters or fitted to a second order polynomial. As a result, the segmentation line is more accurately matched to the microlayer boundary.

In some examples, after the operation 620, a graph search (GS) technique or a Randomized Hough Transform (RHT) technique was used to further strengthen segmentation. For the GS technique, a graph of the segmented image was constructed, each pixel in the image was considered as a node in the graph, and the system calculated an edge between these nodes based on their gray-values and their gradient. Then, when searching for a specific interface, the system only examined the points in this search region, which reduced the search time and increased the accuracy. Once a path of minimum weight was found by the system, it was declared as the interface in this region. The same procedure was done for all interfaces. Thus, in this example embodiment of the GS technique, modifications were done in the construction of the graph such as the definition of the start node and the end done, the edge weights, and the connectivity between nodes.

In RHT technique, the unknown parameters of a model were detected by the system from points potentially fitting the model using a voting scheme. For the RHT technique, a second order polynomial model was used (y=ax²+bx+c with the unknown parameters a, b, and c). The RHT technique then transformed each point from the Cartesian space (x, y) into the parameter space (a, b, c), where points voted for every possible value of the parameters in a finite range. The vote, performed by the system, was done using the gray-value of the point. Finally, points in the parameter space that had maximum votes were chosen as the parameters value for the used model. To speed up the vote process, only the highest gray-value points were used by the system. Thus, in this example embodiment, the system used RHT, a second order polynomial model, and did not use any prior knowledge.

The refinement operations 620 and 622 are optional and may not be performed in some examples. Either way, the resulting segmented image is output at an operation 624, after which control may be passed to operation 410. In some embodiments, the GS technique, the RHT technique, or other techniques may be utilized to perform the segmentation (e.g., without flattening the average image or other images of the cornea).

### Examples

The present techniques were implemented in a number of empirical studies.

In one study, the techniques were applied on OCT images of 5 normal eyes to automatically segment, and create the three-dimensional Endothelium/Descemet's membrane complex (En/DM) microlayer color-coded and bullseye maps. Maps were divided into different regions. En/DM mean thickness in normal subjects was 16.19 μm. En/DM showed thickening towards the peripheral cornea. The mean thickness of the central En/DM was 11±2 μm (Mean and SD), paracentral En/DM region was 12±2.75 μm and the peripheral En/DM was 15.5±4.75 μm. The study showed that in normal Subjects, En/DM showed relative thickening towards the peripheral cornea.

In one study, the techniques were used to evaluate using En/DM three-dimensional microlayer tomography maps to diagnose Fuchs' endothelial dystrophy. We imaged 27 eyes of 23 individuals (11 Fuchs' endothelial dystrophy eyes in 7 patients; 16 control eyes) using OCT. En/DM layer was segmented using the automatic segmentation method. Three-dimensional En/DM Color-coded and bullseye microlayer tomography thickness maps were created and divided into different regions. In Fuchs' endothelial dystrophy eyes, En/DM three-dimensional microlayer tomography maps showed significant thickening as compared to controls. In Fuchs' endothelial dystrophy eyes, mean thickness was 25, 27 & 31 μm in central, paracentral and peripheral zones versus 12, 13 & 15 μm, respectively, in controls (P<0.0001). The En/DM map showed relative thickening towards the periphery in both control and Fuchs' patients (P=0.045 & P<0.0001 respectively). The study showed, for the first time, that En/DM three-dimensional microlayer tomography maps show significant thickening in Fuchs' endothelial dystrophy, as compared to controls.

In one study, the techniques were used to create color-coded and bullseye maps of En/DM layer in patients with graft rejection and compare them to control eyes. The study demonstrated that En/DM three-dimensional microlayer tomography maps show significant thickening in corneal graft rejection as compared to control eyes. In this prospective interventional case series, 22 eyes with corneal grafts post penetrating Keratoplasty (PKP) and Descemet Stripping Automated Endothelial Keratoplasty (DSAEK; 17 clear, and 5 rejected corneal grafts) were imaged using OCT. The microlayers of the cornea were segmented automatically. Color-coded three-dimensional thickness and bullseye maps of the layer were created. With the techniques, we were able to create three-dimensional color-coded microlayer tomography maps and bullseye maps of the layer for all included eyes. The mean thickness of En/DM on the bullseye were 20.15±5.66, 23.16±7.01, 28.57±10.45 versus 41.44±21.96, 47.71±23.45 and 59.20±25.65 μm for central, paracentral and peripheral regions in clear graft versus rejected graft, respectively. The study showed specific thickening in rejected grafts when compared to clear graft.

In one study, the techniques were used to create three-dimensional microlayer thickness color-coded and bullseye maps of the corneal basal epithelial layer (B-Epi) and reported the thickness data of 12 normal subjects. Images were obtained using an OCT and corneal layers were then segmented. A refraction correction algorithm was used to correct optical distortions. Three-dimensional microlayer tomography thickness maps (C-MLT) were generated. One patient with limbal stem cell dystrophy (LSCD) was imaged for comparison. The thickness of B-Epi was found to be uniform between the center, mid-periphery and periphery with means of 12.2+1.8, 12.5+1.9 & 13.3+2.2 μm, respectively. The thickness of the B-Epi positively correlated, showing significance, with corneal epithelium (p<0.0001, R=0.64). Weak correlation between of the B-Epi and the corneal thickness was demonstrated (p=0.003, R=0.2). The patient with LSCD exhibited an attenuated B-Epi and complete absence of the layer in the left eye. The study showed that corneal microlayer tomography 3-D thickness maps (C-MLT) provide a tool to study the basal layer of the epithelium. C-MLT disclosed that this layer is uniform across the cornea, and correlates with epithelial and total corneal thickness. The study showed that patients with LSCD have an attenuated layer.

In one study, the techniques were used to create three-dimensional Bowman's layer microlayer optical coherence tomography maps (e.g., heat maps or bullseye maps) for normal subjects. 13 normal eyes were imaged using OCT. Segmentation method were employed to automatically segmented the microlayers of the cornea. Corneal microlayer surfaces were reconstructed, and a refraction correction algorithm were used to correct optical distortions. Color-coded three-dimensional and bullseye thickness maps of the layer were created. Using our technique, we were able to create the microlayer and bullseye maps of the layer for all included eyes. Bullseye map was divided to different regions (specifically, using the mapping of FIG. 11). The mean thickness data on the bullseye of normal subjects were 19±1, 19±1, 20±2, 20±3, 21±2, 20±1, 20±3, 20±2, 23±2, 24±4, 24±4, 23±3, 24±4, 25±4 um for C1, C2, M1, M2, M3, M4, M5, M6, 01, 02, 03, 04, 05, 06, respectively. Peripheral BL was significantly thicker than the mid-peripheral regions (P<0.001). Both peripheral and middle regions' Bowman's Layer were significantly thicker than the central region's Bowman's Layer (P<0.001). There was a weak positive correlation between Bowman's Layer thickness and total corneal thickness (R=0.3, P<0.001). The study showed that in normal subjects, Bowman's Layer significantly thickens as the layer progresses from the center to the periphery.

In one study, the techniques were used to create 3-Dimensional Bowman's layer microlayer tomography maps (e.g., heat maps or bullseye maps) and evaluated the use of the created maps for diagnosing keratoconus (KC). 30 eyes (15 KC and 15 controls) were imaged using OCT with scanning protocol to image the BL over a 9 mm diameter zone of the cornea. Images were analyzed to segment the Bowman's Layer producing a 9 mm color-coded Bowman's layer microlayer tomography maps. Receiver operating characteristic curves were created to evaluate their diagnostic accuracy. Bowman's Layer three-dimensional microlayer tomography maps disclosed significant Bowman's Layer thinning in KC eyes compared to controls (P<0.001). Bowman's Layer thinning in inferior half of the cornea had excellent accuracy in diagnosing KC with an area under the curve of 1 (P<0.001). Bowman's Layer thickness was less than 14 m in the inferior half of the cornea was 100% sensitive and specific in diagnosing KC. The study showed that Bowman's Layer three-dimensional extra wide microlayer tomography map has excellent accuracy, sensitivity and specificity in diagnosing keratoconus.

In one study, the techniques were used to create Bowman's microlayer tomography 3-dimensional (three-dimensional) maps (e.g., heat maps or bullseye maps) in the diagnosis of subclinical keratoconus (KC). 40 eyes (17 normal and 23 subclinical KC) were imaged using OCT. Subclinical KC was defined as patients with normal clinical examination and Placido topography (TMS-3; Tomey, Erlangen, Germany) but abnormal elevation tomography (Pentacam; Oculus, Wetzlar, Germany). The techniques segmented Bowman's layer (BL). Segmentations were reconstructed to produce Bowman's layer color-coded three-dimensional microlayer thickness and bullseye maps. Bullseye maps were divided into 14 different regions (see, e.g., FIG. 11) and Bowman's layer thickness was calculated for each region and compared between groups. Bowman's layer color-coded three-dimensional microlayer thickness and bullseye maps were successfully created for all studied eyes. In subclinical KC, Bowman's layer color-coded three-dimensional microlayer thickness and bullseye maps disclosed localized relative thinning of Bowman's layer. In subclinical KC, Bowman's layer minimum thickness was significantly less in C1, C2, C5 regions (p<0.01). As such, Bowman's layer color-coded three-dimensional microlayer thickness and bullseye maps techniques may be used for diagnosis of subclinical keratoconus. Bowman's layer color-coded three-dimensional microlayer thickness and bullseye maps disclosed a significant localized relative thinning that can be quantified using our maps.

The study also showed that Bowman's Ectasia index (BEI), an index from the first patent, calculated in each segment of the three-dimensional map was diagnostic of subclinical KC. Bowman Ectasia index (BEI) were calculated for each region and compared between groups. As discussed above, the BEI was defined as minimum thickness of BL in each region of the inferior cornea divided by the mean thickness of BL in the corresponding region of the superior cornea multiplied by 100. The study found that BEI was significantly lower in subclinical KC as compared to normal eyes in region C1, C2, M1, M2, M4, M5, M6, 04 and 05 (70±11, 70±12, 72±12, 71±11, 73±13, 62±19, 71±13, 66±19, 60±20 μm vs. 83±8, 83±11, 80±9, 81±9, 82±8, 80±11, 80±12, 78±15, 78±20 μm; P<0.05).

In one study, the techniques were used to generate three-dimensional maps of the Bowman's layer for a patient with post-refractive surgery ectasia. The techniques were used to analyze images and segment the Bowman's layer and produce three-dimensional color-coded Bowman's layer tomography maps (e.g., heat maps or bullseye maps). The three-dimensional color-coded and bullseye map of Bowman's layer disclosed pathological thinning of the layer. Thus, the study showed that three-dimensional Bowman's microlayer tomography map may be used in diagnosing post-refractive surgery ectasia.

In another study, we imaged patient with dry eye syndrome and normal patient using OCT. Three-dimensional color-coded and bullseye maps were generated using the present techniques. The Epithelial microlayer corneal tomography map disclosed that the epithelium is highly irregular as compared to the normal subject.

The techniques herein were also used to examine, for the first time, collagen crosslinking (CXL). CXL is a treatment modality for progressive corneal ectasia. CXL has proven to strengthen the corneal tissue by forming new covalent bonds between collagen fibers. It was noted that the treatment leads to development of a demarcation line in the cornea, that is, hyper-reflective areas of the cornea that are said to represent the transition zone between the crosslinked and the untreated cornea. That transition zone is a measurement of the depth of CXL treatment into the cornea and thus a measurement of its effectiveness. The present techniques were used to create a three-dimensional total corneal collagen crosslinking demarcation band microlayer tomography maps (e.g., heat maps or bullseye maps), which our studies show correlate with the localized effect of treatment on the cornea.

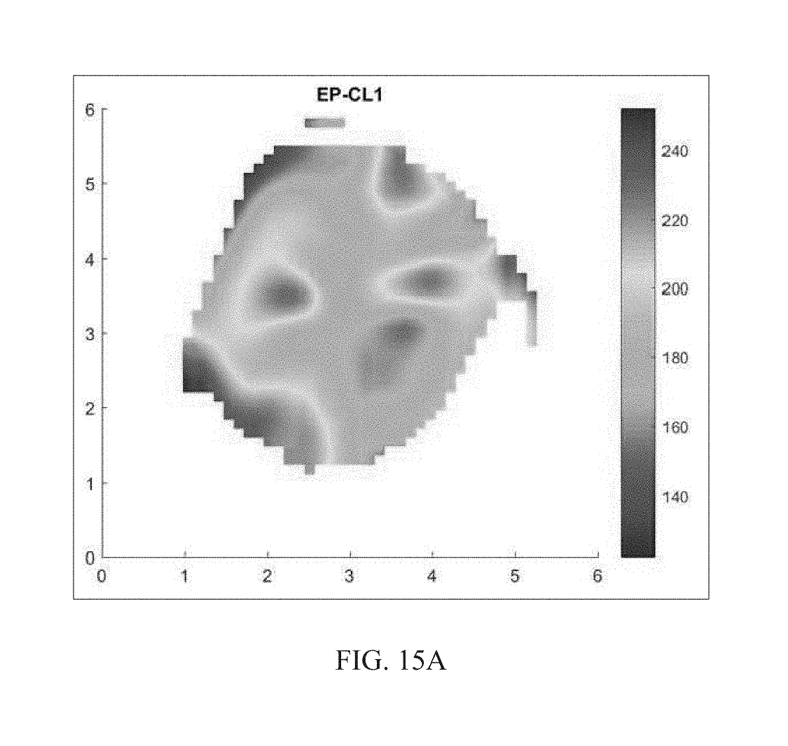

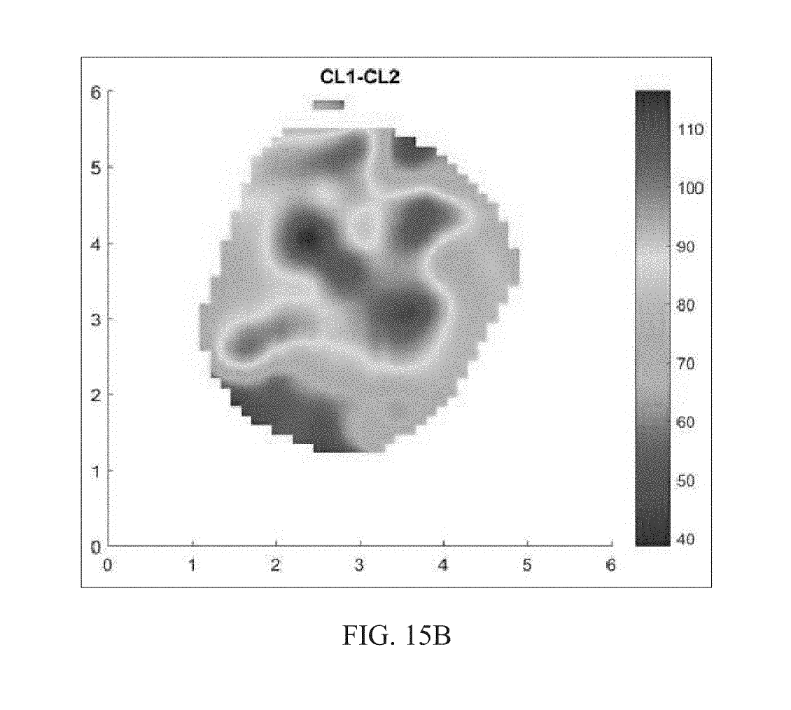

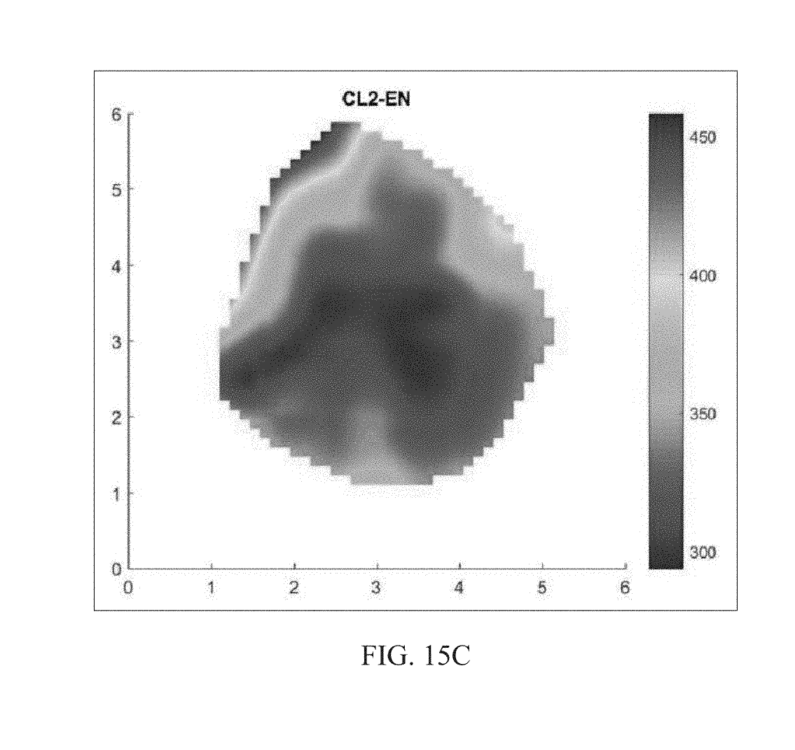

In one study, 18 eyes with progressive keratoconus underwent corneal CXL. In 1 month postoperatively, OCT maps were captured and analyzed with the system to create three-dimensional corneal collagen crosslinking demarcation band maps (CXL-OCT), see, e.g., FIGS. 15A-15C. Correlation between demarcation band characteristics on CXL-OCT and corneal curvature changes captured using Pentacam tomography were evaluated.

Using the present techniques, we were able to generate CXL-OCT maps for all patients. The mean thickness (FIG. 15B) and depth (FIG. 15A) of CXL demarcation bands were 77±35 μm and 279±82 μm, respectively. CXL demarcation bands maps were significantly thicker in central cornea compared to paracentral and peripheral cornea (91±31, 74±34, and 63±35 μm, respectively; p<0.01) (FIG. 15B). There was no significant difference between the depth of CXL demarcation band maps in different corneal regions (FIG. 15A). Significant positive correlation was noted between CXL demarcation band depth and postoperative flattening effect in the inferior cornea (R=0.4, P<0.05) as disclosed using the CXL-OCT. Thus, the study showed that the deeper the localized demarcation band on the CXL-OCT maps, the more the localized postoperative flattening effect is.

As introduced above, the imaging system may pre-process images to remove artifacts and increase the signal to noise ratio. In various embodiments, the pre-processing may rely on statistics of the image rather than using general statistics or constraints. The imaging system may identify image artifacts to remove them. Image artifacts may misguide a segmentation algorithm as such artifacts may have large gray values similar compared to gray values for actual layers. As disclosed herein, an automated method may be implemented by the imaging system based on a vertical projection of the image. The method may rely on a constant pattern in the vertical projection of the images, for example. Once the pattern is identified, the imaging system may remove portions of the image containing the artifact. The imaging system may then extract the most prominent points from each A-scan. The imaging system may further apply local processing as it judges each pixel in its context to enhance microlayers detection in images with low SNR. In one application, points that satisfy the following criteria are kept and identified as prominent points for each A-scan A(z):

$$A ( z ) = \{ \begin{matrix}A ( z ) ; & A ( z ) \geq {μ}_{A} + α ( z ) {σ}_{A} \\ 0 , & otherwise\end{matrix}$$

where μ_{A} and σ_{A} are the mean and the standard deviation of A(z) respectively and a(z) is given by

$$α ( z ) = \{ \begin{matrix}\vert \frac{A ( z ) - {μ}_{A}}{{σ}_{A}} \vert ; & \vert \frac{A ( z ) - {μ}_{A}}{{σ}_{A}} \vert \geq {μ}_{α} \\ {μ}_{α} ; & otherwise\end{matrix}$$

where μ_{α} is the mean of and a(z).

The resultant image may then be processed row-wise using only non-zero values and only the prominent row values are kept. This double processing suppresses artifacts. After prominent points extraction, a median filter, such as a 3×3 median filter, may be applied one or more times, such as twice, to reduce the scattered bright points also known as speckle noise.

The tear film is a liquid layer that bathes the ocular surface of the eye including the cornea and the conjunctiva. It creates a smooth layer that covers the entire ocular surface. It is the anterior most interface of the ocular surface. As introduced above, dry eye syndrome (DES) affects more than 20% of the population and thus is a public health problem. In DES, the decrease in the quality or quantity of the tear film breaches the protective function of tears and that results in damage to the ocular surface especially its epithelium. This damage results in DES signs and symptoms.

Despite the prevalence of DES, current DES diagnostic techniques generally show less than optimal association with patient symptoms. Without being bound to theory, a reason for this is believed to be because current DES diagnostic techniques may suffer from poor standardization and are affected by multiple confounding factors that are difficult to control. According to various embodiments described herein, a successful strategy for diagnosis of dry eye focuses on detection of the injury on the ocular surface and which may include one or more of spatially describing, quantifying, or visually rendering the detected injury along the ocular surfaces. With respect to current imaging technology, segmentation of the epithelium layer typically combines both the epithelium layer and the tear film layer. That is, current imaging technology does not delineate the tear film from the epithelium, but rather views the epithelium layer not a separate corneal structure but as a layer that includes the tear film. However, as herein described, resolution of the true anterior surface of the epithelium layer may greatly improve diagnostic capabilities of imaging technology and provide stronger correlation with patient systems. Prior to the present disclosure, imaging strategies for automatically delineating the tear film from the epithelium of the ocular surface have not been reported. Thus, prior to the present disclosure, researchers and practitioners have not been able to map the true surface of the epithelium and the tear film.

As introduced above, the systems and methods described herein may be utilized for one or more of improved diagnosis, treatment, monitoring, or evaluation of dry eye syndrome (DES). For example, the systems and method may provide for enhanced mapping of the tear film and the epithelium of the ocular surface. In particular, the systems and methods described herein may be utilized to unmask anterior surface irregularities. The imaging system may be employed to examine the ocular surface and automatically detect the tear film and the true epithelial surface to disclose the injurious effect of DES on the ocular surface and to quantify it. That injurious effect is believed to manifest as an irregular anterior epithelial surface as compared to normal subjects who have a smoother surface. From a patient prospective, those irregularities are detected by corneal rich sensory nerves and then translated into discomfort and pain. However, the irregularities of the epithelium are smoothened by the tear film overlying it and current techniques fail to capture these irregularities of the ocular surface in their full extent because they do not delineate the two layers and segment them.

In various embodiments, the imaging system may be configured to evaluate the ocular surface of an eye including the epithelium, tear film, or other layer. In one example, the imaging system is configured to segment images of the ocular surface of the eye to identify the epithelium layer or tear film layer, which may include each layer separately segmented. The epithelial layer and tear film layer may be segmented, for example, automatically, to unmask the true surfaces and thickness of the layers. Irregularities along the anterior interface of the epithelium may be measured after separating the tear film from the epithelium. One strategy is to use the thickness of the epithelium and use its variations as a measurement of irregularity. In instances where the posterior epithelium surface includes irregularities, however, this may introduce a confounding factor. According to various embodiments, the imaging system may be configured to detect irregularities along the surface of the epithelium, tear film, or other layer. For example, the anterior surface of the epithelium, tear film, or other layer may be compared to a smooth model that is created to fit the cornea being studied. The difference in pixels between the smooth model and the true surface of the respective epithelium, tear film, or other layer may be measured.

The imaging system may create maps of the tear film and true epithelium (e.g., three-dimensional maps or other maps). For example, the imaging system may determine one or more of thickness, hyper-reflectivity, or irregularity data from the segmentation of each layer and, using such data, generate one or more three-dimensional maps. The three-dimensional maps may include thickness, hyper-reflectivity, or irregularity maps of the tear film and epithelial surface of the ocular surface. In these or other embodiments, any of the three-dimensional maps may include heat or bullseye mapping schemes. In one example, pixel differences between a true anterior surface and a smooth corneal model may be incorporated into a map that may be displayed to a user, e.g., with in a heat or bullseye map.

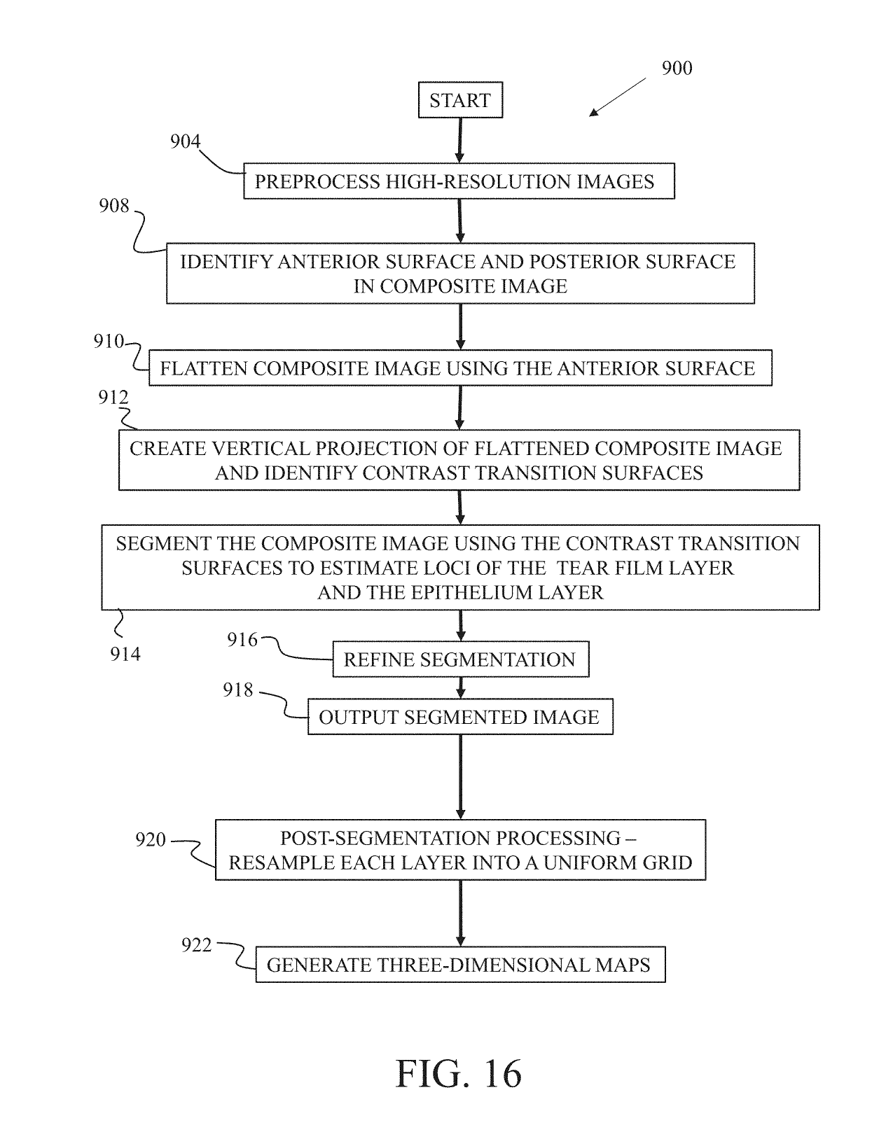

The system may segment and identify the epithelium and tear film layers, including thickness, irregularities, or hyper-reflectivity consistent with the various methods described herein. FIG. 16 illustrates one method 900 for automatically identifying and segmenting the epithelium and tear film layers according to one embodiment. The imaging system may include an imaging device or may receive cornea images for analysis taken by an imaging device, as described above.

At operation 904, the imaging system may preprocess images to generate composite images that improve, for example, signal to noise ratio. Such preprocessing can include image registration and averaging to increase segmentation accuracy. Preprocessing may be performed by any suitable method. For example, preprocessing may be performed according to preprocessing methods described herein such as segmentation-based registration and those described with respect to FIG. 4, 9, or 10 or elsewhere herein. In another example, the imaging system may be configured to perform one or more of the preprocessing operations, which, in some embodiments may be selectable by a user. According to various embodiments, preprocessing 904 is optional. For example, the imaging system may receive preprocessed images. In some embodiments, preprocessing may not be performed, and the process proceeds to operation 908 using raw images. The images will typically include images of multiple cross-sections of the cornea that will be used to develop three-dimensional mapping of one or more layers.

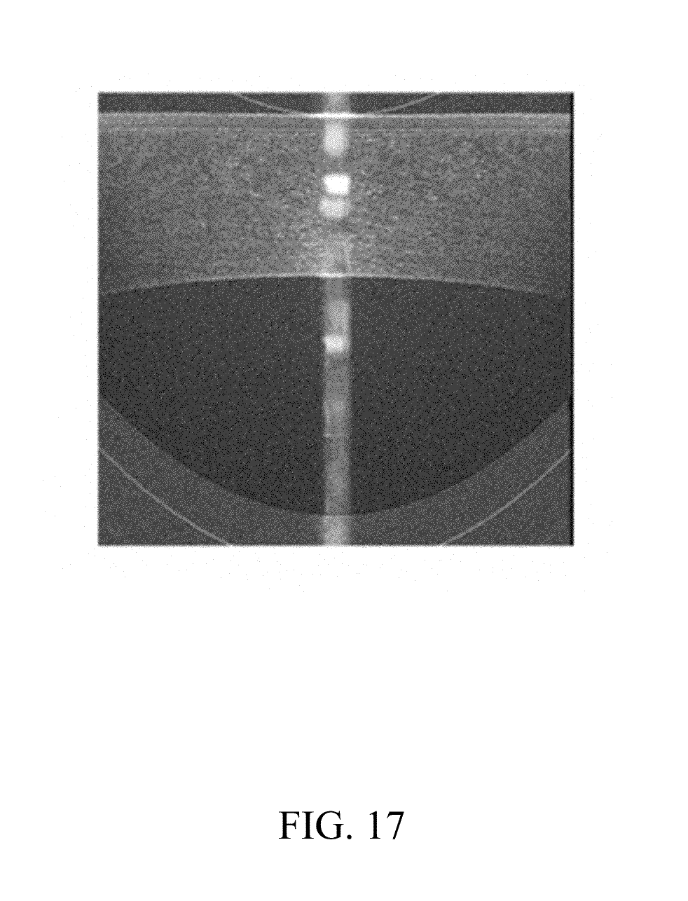

With continued reference to FIG. 16, imaging system is configured to process the ocular surface images to automatically identify and segment the epithelium layer and tear film layer. At operation 908, the imaging system identifies an anterior surface and a posterior surface of the high-resolution composite image, or raw image if pre-processing is not performed or does not produce a composite image. The anterior surface is typically the anterior ocular surface and the posterior surface is typically the posterior surface of the endothelium; however, other surfaces may be used, e.g., the posterior surface of the Bowman's layer, stroma, Descemet's membrane, or endothelial layer. In some embodiments, identification of a posterior surface is optional. At operation 910, the imaging system flattens the composite or raw image, as the case may be, using the anterior surface. An example composite image flattened using the anterior surface is shown in FIG. 17.

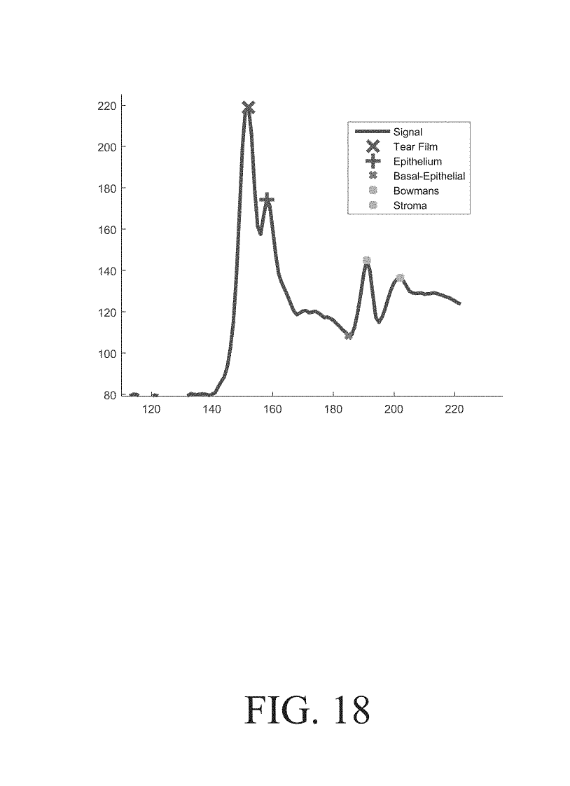

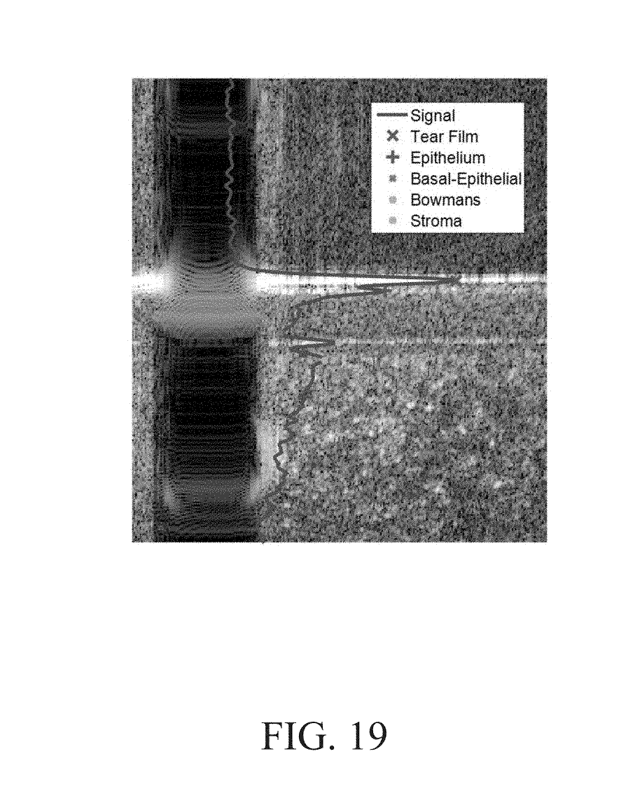

Continuing method 900, the imaging system may further generate a segmented composite image. At operation 912, the imaging system may process the flattened image by creating a vertical projection of the flattened image using the anterior surface, an example of which is provided in FIG. 18, identify contrast transition surfaces corresponding to interfaces between the tear film and epithelium. The imaging system may also identify contrast transition surfaces corresponding to the basal epithelium, Bowman's layer, or other layer. The vertical projection may include vertical projection of A-scans or axial segments of the image. In one embodiment, identification of contrast transition surfaces may include peak detection. In the example vertical projection of the anterior-flattened image illustrated in FIG. 18, the tear film “X” is identified as the first peak and the epithelium “+” is identified as the second peak. The Bowman's layer boundaries (solid circles) are identified as the first two peaks after the peak corresponding to the epithelium “X”, with the second Bowman's boundary (second solid circle) corresponding to a boundary of the stroma. The basal-epithelium “x” is identified as the minimum point between the peak corresponding to the epithelium “X” and the first Bowman's layer boundary peak (first solid circle). FIG. 19 illustrates the vertical projection of FIG. 18 overlaid along the central portion of the composite image, showing correspondence between the vertical projection and the image.

Alternatively or additionally to peak detection, contrast transition surfaces may be identified using gradient in the vertical projection. In one example, a contrast identification-based algorithm, such as an algorithm that identifies gradient changes from dark to bright or bright to dark, may be used to identify contrast transition interfaces for estimation of layer loci. A gradient analysis can be performed on the ocular surface images that may, for example, identify gradient changes of a threshold amount to detect the tear film and the epithelium. Graph search theory algorithms can also be used to automatically segment the ocular surface including the cornea, which may further include the conjunctiva, by detecting the interfaces between the tear film, epithelium, basal epithelium or Bowman's layer. In some examples, particular image filters may be further combined with the image processing to more accurately identify transitions from the vertical projection.

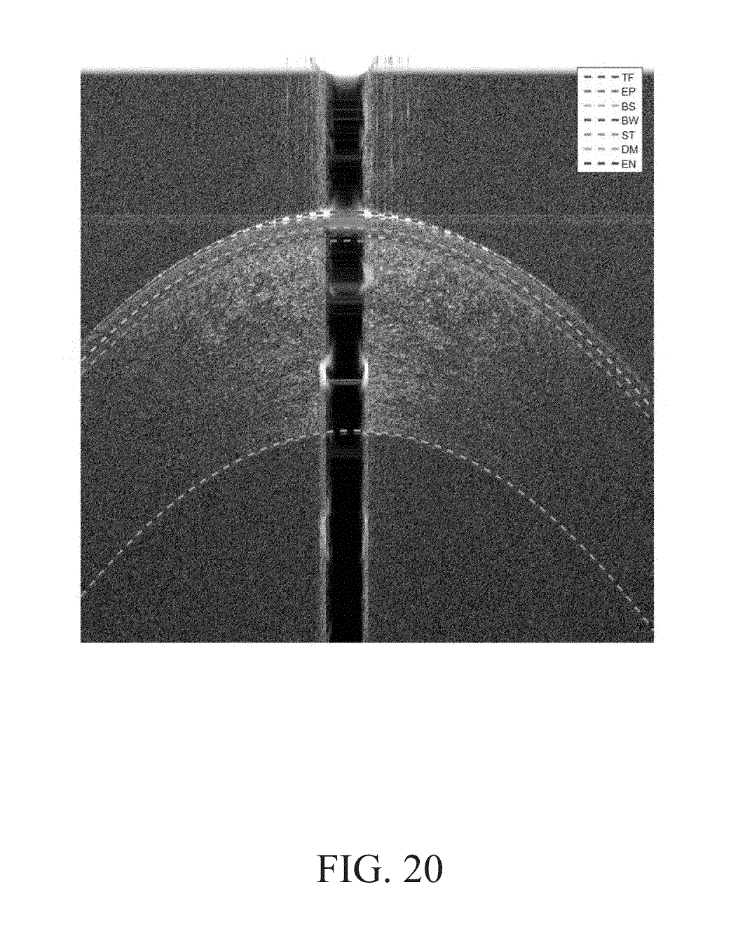

At operation 914, the imaging system may segment the composite image using the contrast transition surfaces to estimate loci of the tear film layer and the epithelium layer. The segmentation may produce a segmented composite image. For example, the estimated loci of the tear film or anterior boundary thereof may be segmented as shown by segment line “TF” in FIG. 20 corresponding to peak “X” in FIG. 18. The estimated loci of the epithelium or anterior boundary thereof may be segmented as shown by segment line “EP” in FIG. 20 corresponding to peak “+” in FIG. 18. The epithelium may be segmented for mapping separate from the basal epithelium by identification and segmentation of the basal epithelium (see, e.g., segmentation line “BS” in FIG. 20 corresponding to valley “x” in FIG. 18). The epithelium may also be segmented for mapping together with the basal epithelium by identification and segmentation of the Bowman's layer (see, e.g., segmentation line “BW” in FIG. 20 corresponding to the first peak identified by a solid dot following the basal epithelium valley “x” in FIG. 18). In various embodiments, the imaging system may segment additional microlayers or combinations of layers as described elsewhere herein.

In various embodiments, the imaging system may be further configured to refine the estimated segmented microlayer loci. The imaging system is preferably configured to perform automatic refining segmentation. With continued reference to FIG. 16, method 900 may further include refining segmentation at process 916. In some embodiments, method 900 may not include refining segmentation or refining segmentation may be optional. The segmented image may be outputted at process 918.

Refining segmentation 916 may be carried out according to any suitable refining operations, such as those described herein. For example, refining segmentation may utilize one or more segmentation techniques. Graph search theory (GS), for example, can be used to refine a segmentation line to that which more closely matches the respective tear film, epithelium, or other layer. In one application, the GS technique be applied such that each pixel in the image is considered a node in the graph. The imaging system may further calculate an edge between the nodes based on the gray-values and gradient of the pixels. The imaging system may identify a path of minimum weight and declared the path the interface in the region.

Additionally, or alternatively, Randomized Hough Transform (RHT) technique may also be used to further strengthen segmentation. In one application of RHT technique, the imaging system detects unknown parameters of a model from points potentially fitting the model using a voting scheme. In an example of the RHT technique, a second order polynomial model may be used (y=ax2+bx+c, with the unknown parameters a, b, and c). The RHT technique may then be used to transform each point from the Cartesian space (x, y) into the parameter space (a, b, c), where points voted for every possible value of the parameters in a finite range. The vote, performed by the imaging system, may be done using the gray-value of the point. Points in the parameter space having maximum votes may then be chosen as the parameters value for the used model. In some embodiments, to speed up the vote process, only the highest gray-value points are used by the imaging system. Thus, in the above example embodiment, the imaging system may use RHT, a second order polynomial model, and not use any prior knowledge. Another technique to refine segmentation may include conducting a local search for each point of the estimated loci of respective tear film, epithelium, or other layer. In various embodiments, refining segmentation may include one or more of processes 406, 620, or 622 as described with respect to FIG. 8 and FIG. 10.

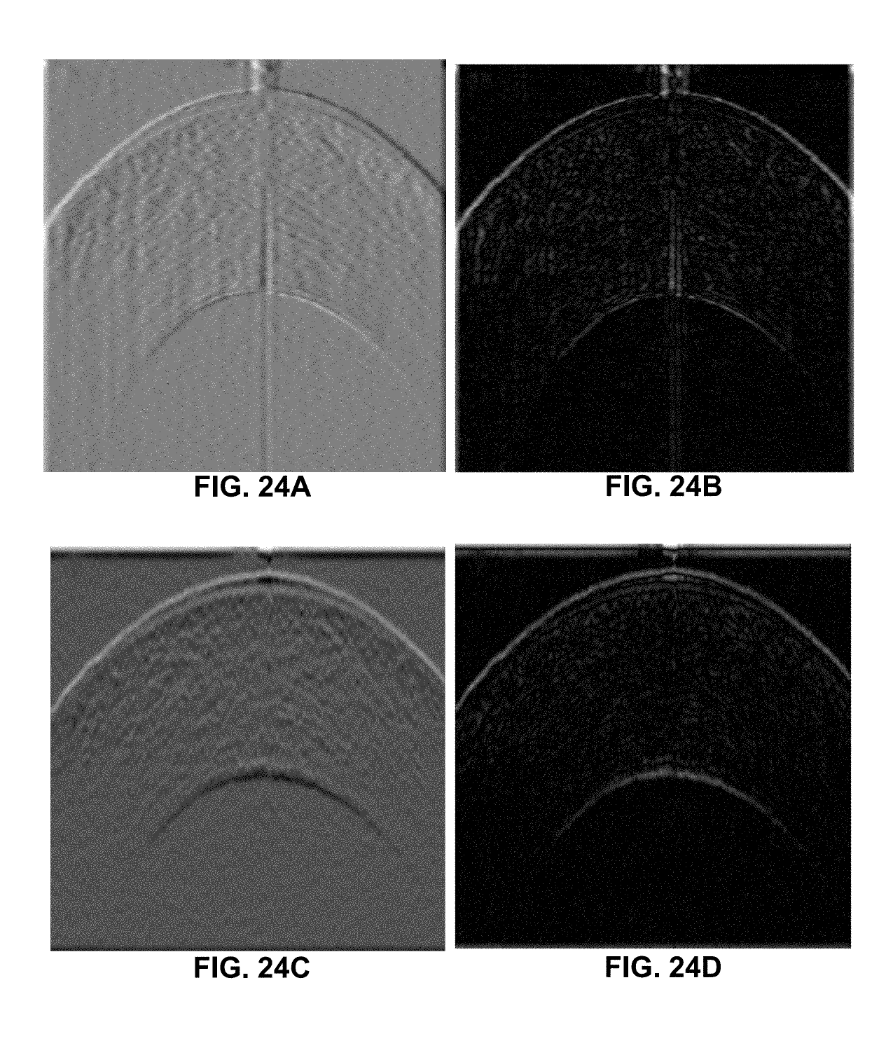

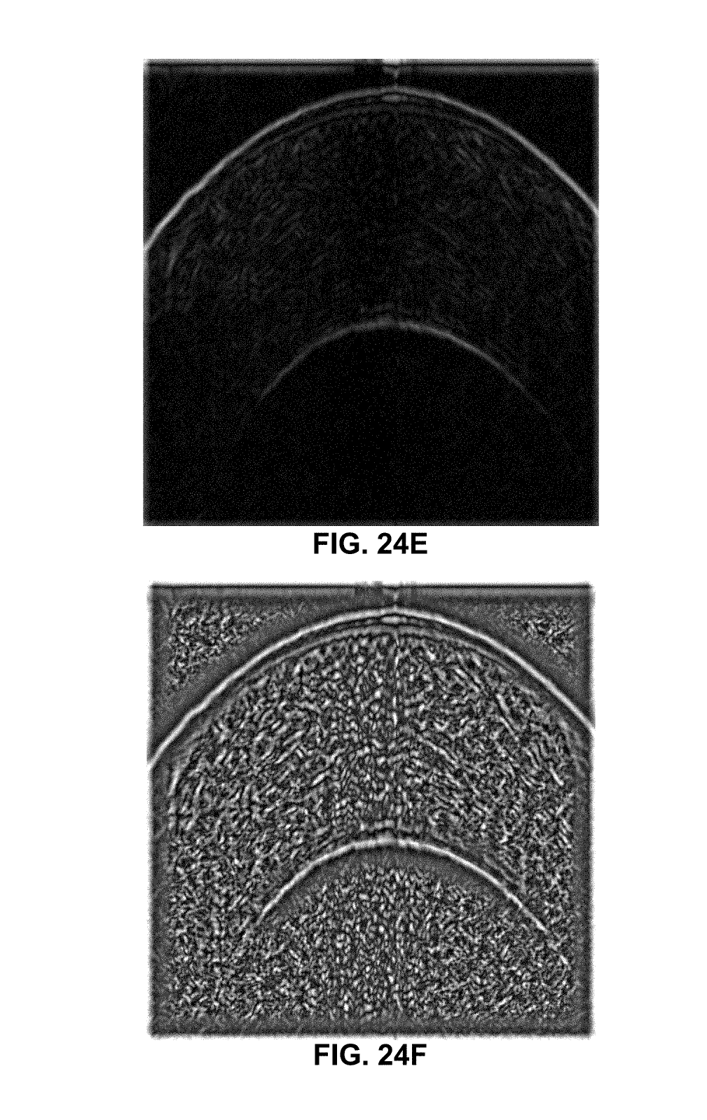

In some embodiments, generation of a map related to a cornea does not involve one or more of the flattening, averaging, or reflectivity profile techniques described above. As an example, the horizontal gradient Gₓ(x,y) shown in FIG. 24A was obtained by filtering a smoothed image using the horizontal filter given by [−1 0 +1] where 1 is a row vector of ones of length 15. The absolute of horizontal gradient Gₓ(x,y) is shown in FIG. 24B. The vertical gradient G_{y}(x,y) of the smoothed image shown in FIG. 24C was obtained by filtering the smoothed image using the vertical filter given by [−1 0 +1]^{T} where l is a row vector of ones of length 15 and T is the transpose operator. The absolute of the vertical gradient G_{y}(x,y) is shown in FIG. 24D.

The final gradient image may be obtained as a weighted sum of the absolutes of the horizontal and vertical gradients as shown in FIG. 24E, and it is given by

G(x,y)=W(x)|Gₓ(x,y)|+|G_{y}(x,y)| (1)

where W(x) is an inverted Gaussian function that is 0 at the center and 1 at the sides. To enhance weak edges, G(x,y) may be locally normalized using its local statistics. The locally normalized gradient g(x,y) image is given by

$$g ( x , y ) = \frac{G ( x , y ) - {μ}_{local} ( x , y )}{{σ}_{local} ( x , y )} \qquad ( 2 )$$

where μ_{local}(x,y) and σ_{local}(x,y) are the local mean and the local standard deviation at the location (x,y), respectively. Finally, g(x,y) was normalized between 0 and 1 as shown in FIG. 24F.

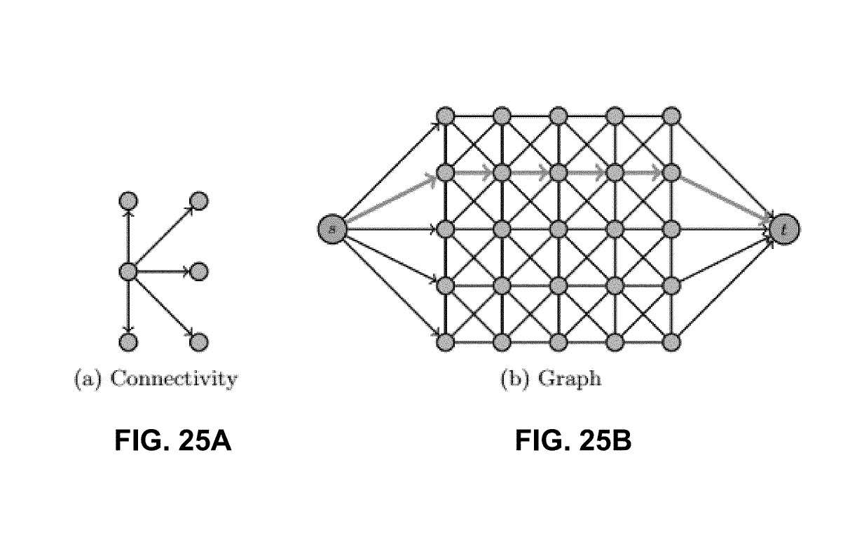

In some embodiments, corneal boundaries may be segmented via use of a graph framework. As an example, a directed graph G(V, E) may be constructed for an image of a cornea, where V is the set of graph vertices (e.g., image pixels and a source s and target t vertices), and E is the set of graph edges (e.g., neighborhood edges and terminal edges). As shown in FIG. 25A, each vertex may be connected to its neighboring vertices using 5-connectivity neighborhood. As shown in FIG. 25B, the source vertex s may be connected to the vertices of the leftmost column of the image, and the target vertex t may be connected to the vertices of the rightmost column of the image. In the first graph-search stage, the initial segmentation of the epithelium layer (EPL) and endothelium layer (ENL) may be obtained using the gradient information. The edge energy E_{ab}, between two vertices a and b, may be defined as

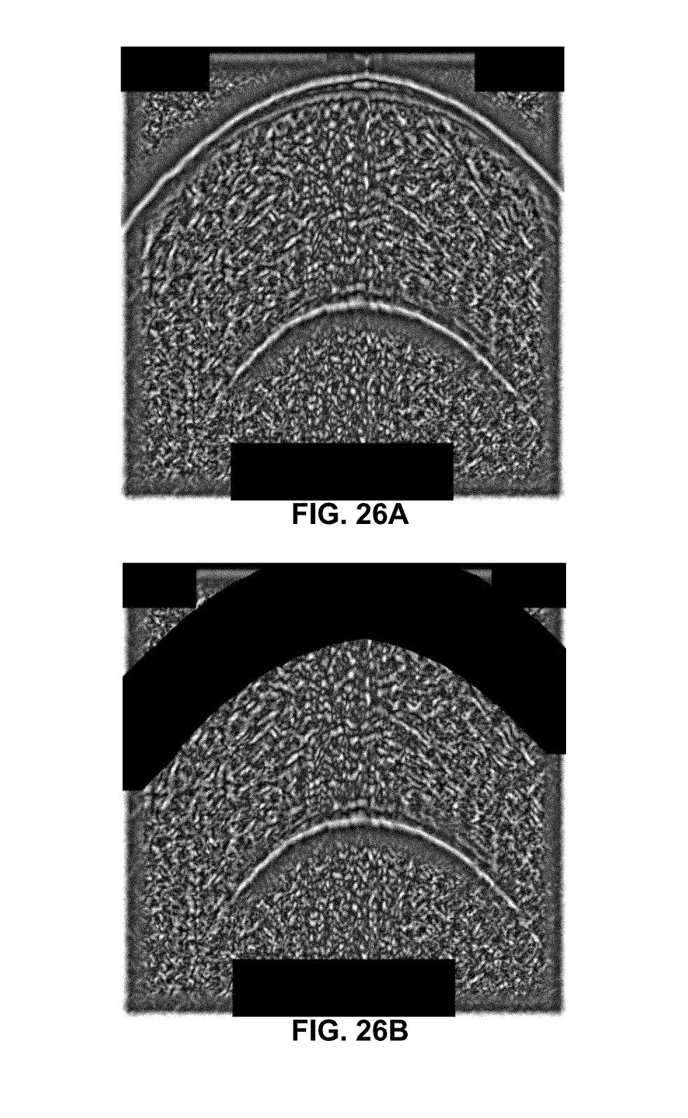

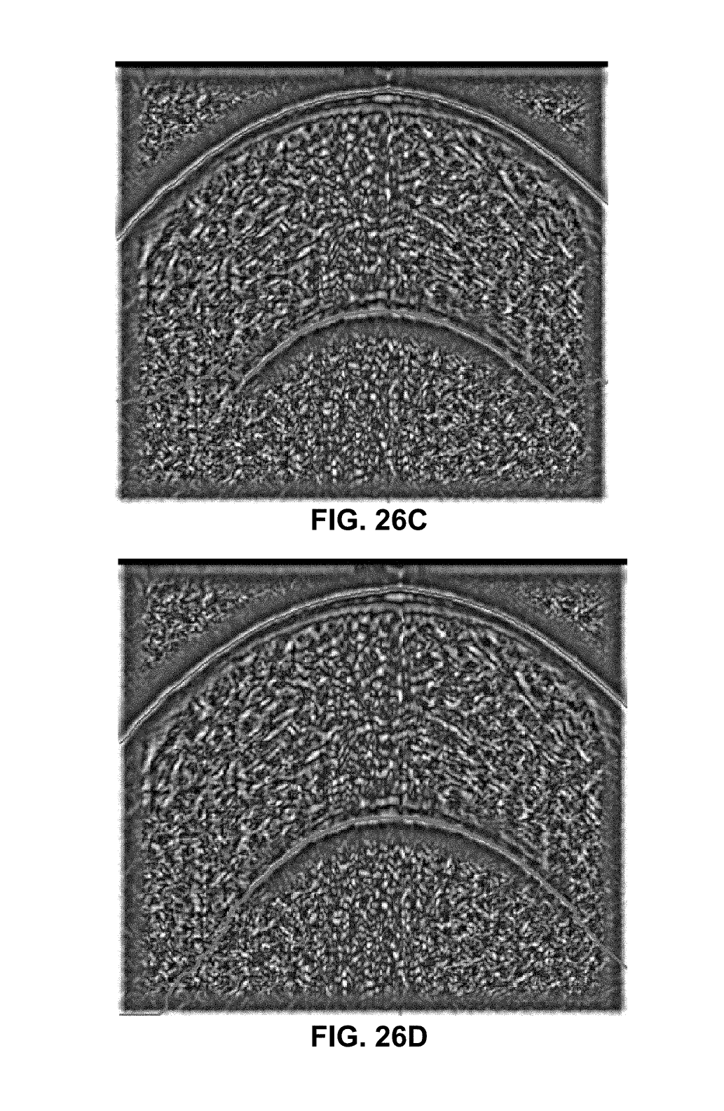

E_{ab}=E_{grad} (3)

where E_{grad} is the gradient energy. The gradient energy E_{grad} may be defined as

$${E}_{grad} = {e}^{( - \frac{{g}_{a}^{2} + {g}_{b}^{2}}{{σ}^{2}} )} \qquad ( 4 )$$

where gₐ and g_{b} are the normalized gradient values at the vertices a and b, respectively and sigma is a constant which was set to 1. Some artifacts were present at the top and bottom edges of the gradient image due to image filtering. Therefore, to prevent the graph search from following those edges, the continuity of these edges may be cut by removing the top left corner, top right corner, and bottom central region from the search region as shown in FIG. 26A. This initial search region, for example, is used to search for the first boundary. After detecting the first boundary (e.g., not necessarily the EPL), the search region may be updated by removing the detected boundary and its vicinity as shown in FIG. 26B. The detected boundaries may be ordered according to their relative locations. An example of the initial segmentation is shown in FIG. 26C.

The initial segmentation of the EPL and ENL usually has artifacts at the peripheral parts. Therefore, in some embodiments, a second graph-search stage may be performed to correct the initial segmentation. In the second graph-search stage, directional information derived from the initial segmentation may be used to guide the segmentation at the peripheral regions with low SNR. A new edge energy function may be defined as

E_{ab}=(E_{grad}+αE_{dir})Eₚₑₙ (5)

where E_{grad} is the gradient energy, E_{dir} is the directional energy, Eₚₑₙ is a penalty energy and α is a weighting factor which was set to 2. The E_{grad} is given by Equation 4. The directional energy E_{dir} may be defined as

E_{dir}=|θₚₒₗ−θ_{ab}| (6)

where θₚₒₗ is the directional angle of a second order polynomial fitted to the boundary and θ_{ab} is directional angle of the edge from vertex a to vertex b. For the ENL, the newly detected EPL may be used to define the directional angle (i.e. θ_{EPL}). The penalty energy Eₚₑₙ may be defined as

Eₚₑₙ=γ|xₐ−x_{b}|+|yₐ−y_{b}| (7)

where γ is a constant and it was set to 3. It may be added to encourage vertical movement to capture vertical edges. The second stage segmentation result is shown in FIG. 26D.

E_{dir}=|θₚₒₗ−θ_{ab}| (8)

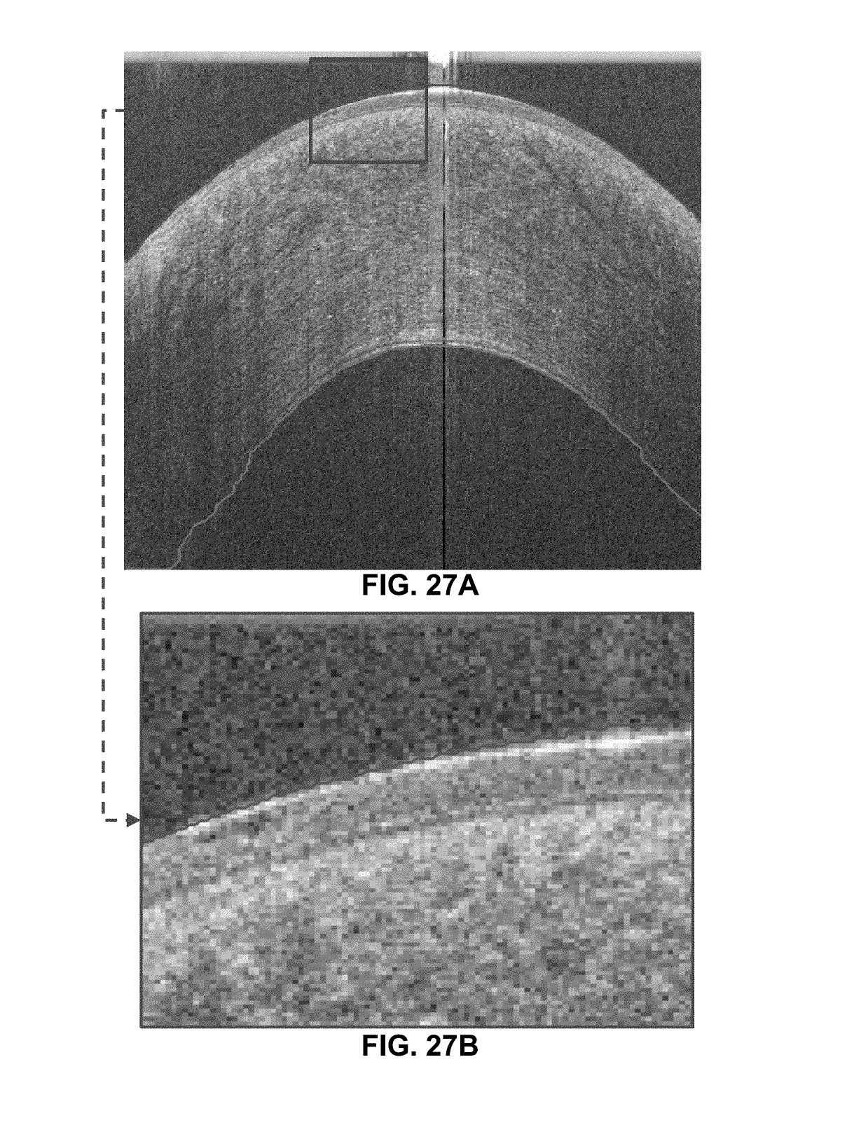

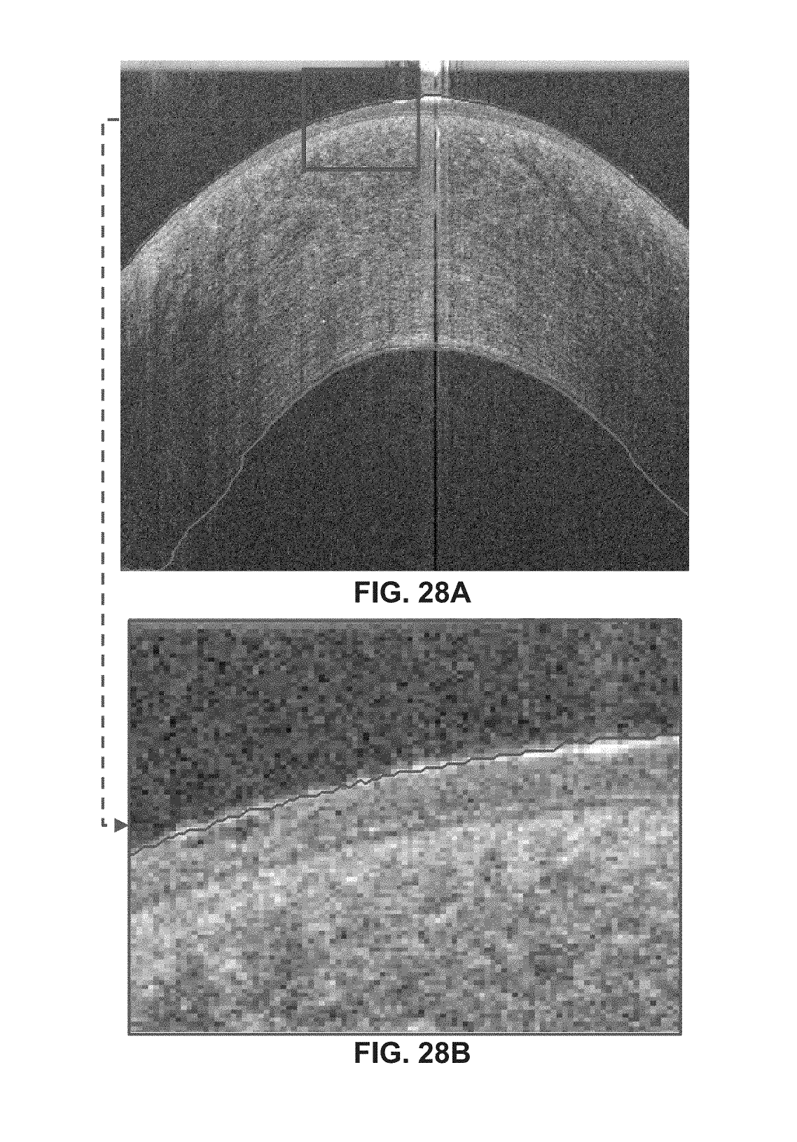

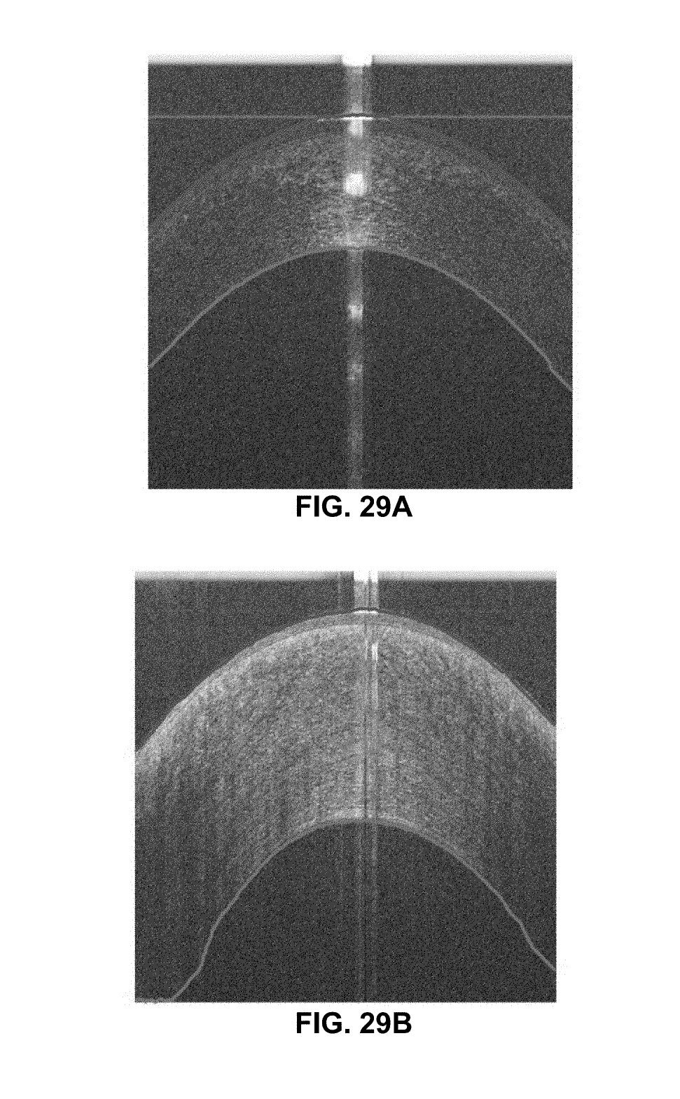

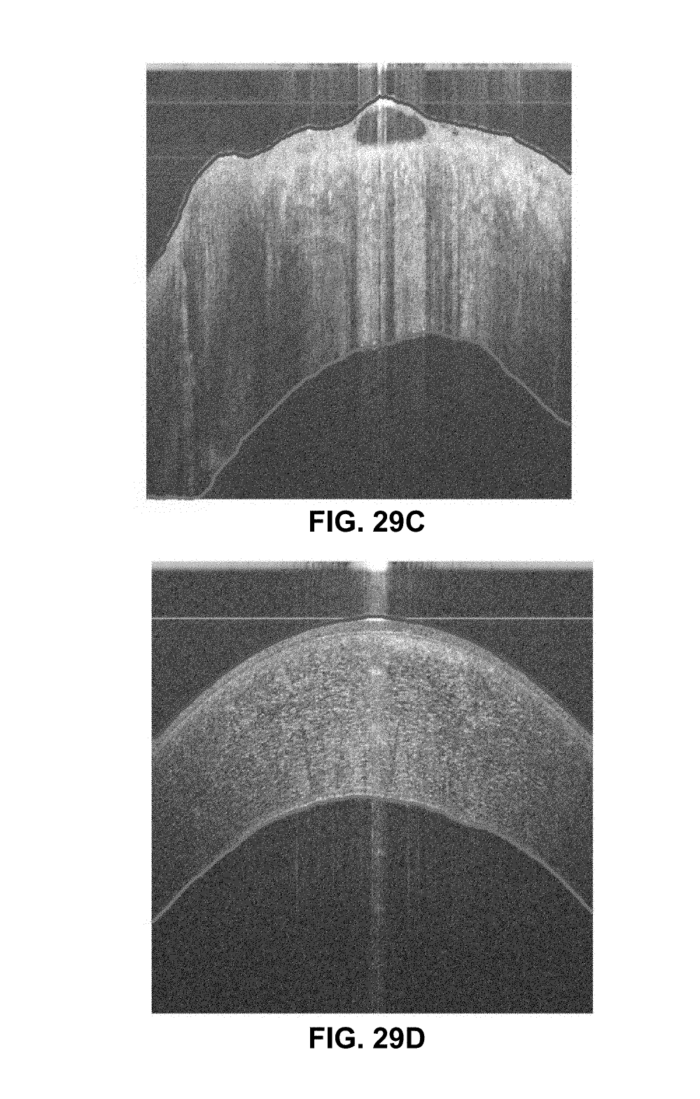

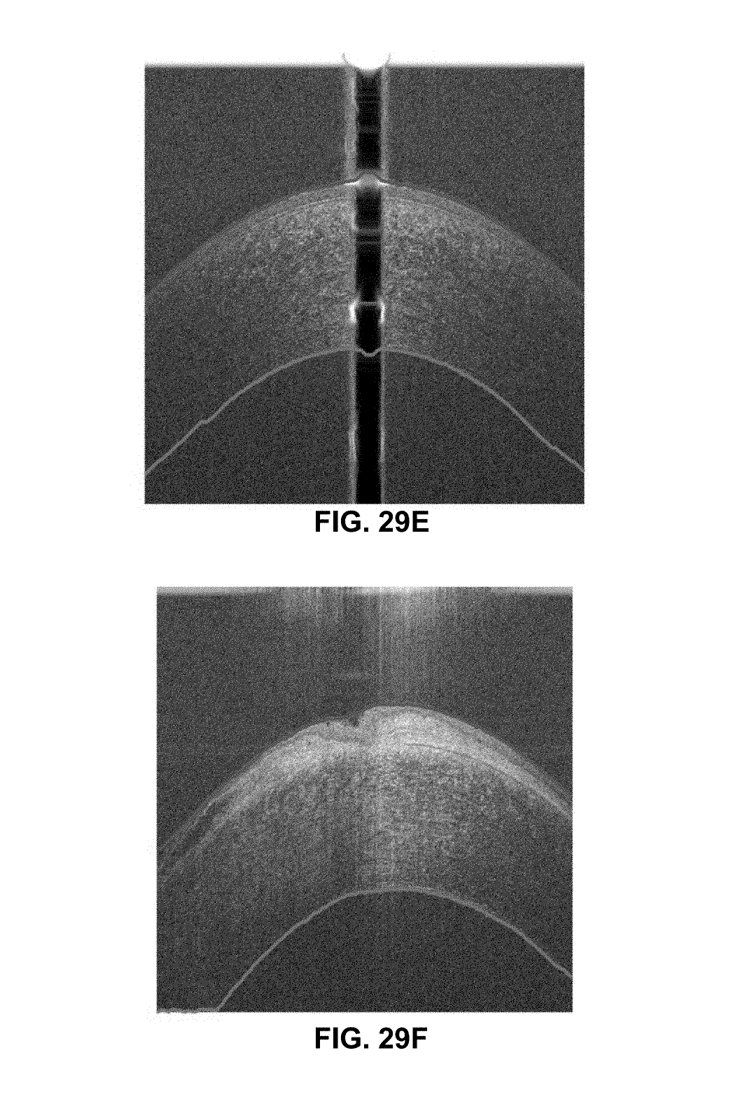

The segmented EPL and ENL may not be aligned with the boundaries in the original OCT image as shown in FIGS. 27A and 27B. Therefore, in some embodiments, a third graph-search stage may be performed around each boundary within 4-pixel window (or other window corresponding to a different number of pixels), to align the segmentation with the boundary as shown in FIGS. 28A and 28B. A new edge energy may be defined as

$${E}_{ab} = {E}_{int} = {e}^{( - \frac{{I}_{a}^{2} + {I}_{b}^{2}}{{σ}^{2}} )} \qquad ( 9 )$$

where Iₐ and I_{b} are the grayscale values of the original image at vertices a and b, respectively and sigma=1. Other examples of segmentations using the proposed methods are shown in FIGS. 29A-29F.

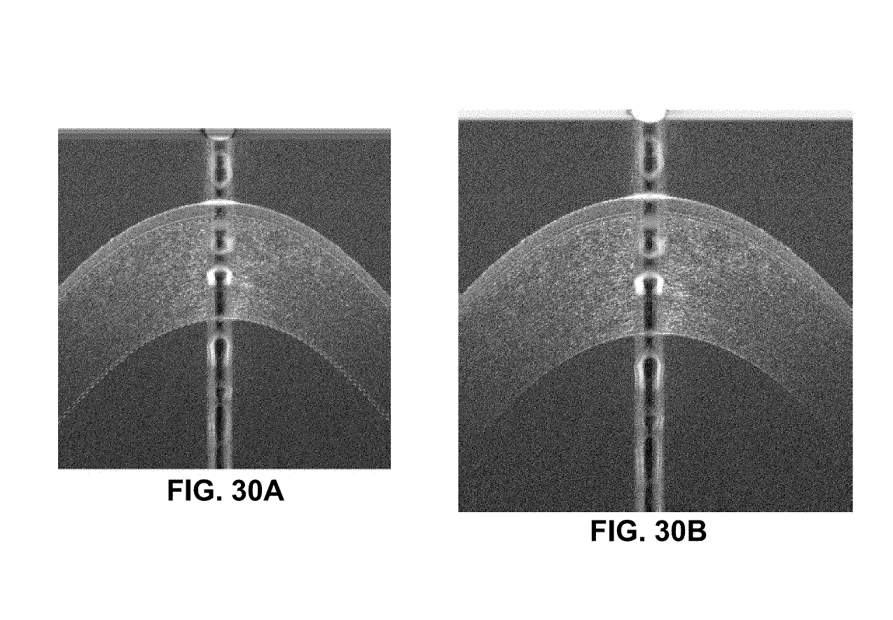

In some embodiments, as indicated herein elsewhere, a double flattening technique may be used to search for the inner layers using our graph search method. As an example, one or more images of the corneal epithelial layer may be flattened, and the flatten images may be used to search for the basal-epithelial, the Bowman's layer, and the stroma. One or more images of the corneal endothelium may be flattened, and the flattened images may be used to search for the Descemet's membrane. Examples of the segmented layers are shown in FIGS. 30A and 30B. FIG. 30A shows an example of an OCT image with the segmentation of the inner layers overlaid on the OCT image. FIG. 30B shows an example of the same OCT image without the segmentation of the inner layers.

FIG. 20 illustrates an example segmentation that includes the tear film (TF) and the epithelium (EP) layers. The epithelium (EP) may be segmented separate from the basal epithelium by identification of the basal epithelium contrast transition surface or together with the basal epithelium by identification, for example utilizing the Bowman's layer contrast transition surface. Separate segmentation may be used to map the layers separately as described below and elsewhere herein. The segmentation shown in FIG. 20 also includes the segmentation at the basal-epithelium (BS), Bowman's Layer (BW), stroma (ST), Descemet's membrane (DM), and endothelium layer (EN), one or more of which may be optional when segmenting the tear film, epithelium, or other layer. The segmentation may be presented to the machine operator to allow the operator to review the segmented images and make changes as appropriate.

With continued reference to FIG. 16, following segmentation, which may include refining segmentation, the imaging system may conduct post-segmentation processing at process 920. In various embodiments, post refinement processing 920 may include one or more of operations 408-414 as described with respect to FIG. 8. Post-segmentation may include aligning the segmented images with segmented images corresponding to adjacent corneal sections, e.g., aligning image data points identified for layers during segmentation that are representative of layer surfaces, interfaces, or boundaries from images of multiple cross-sections or sections, and mapping the segmented images, or imaged data obtained therefrom, into three-dimensional points across the cornea. The three-dimensional points may comprise a cloud of points within a uniform grid represented in data. The cloud of points may be further interpolated to produce a representative layer surface of one or more layers. It is to be appreciated that while the present disclosure generally describes assembly of sectional images into three-dimensional points as being those corresponding to layers as identified in cross-section images, the present techniques may be applied to other section images or orientation of images in which depth may be determined and extrapolated to map and generate three-dimensional maps as described herein. In the illustrated example, post-segmentation processing 920 includes resampling each layer into a uniform grid. The layers may be represented in a three-dimensional point cloud of data points in the uniform grid in which the three-dimensional relationships of the data points may be represented. The imaging system may further interpolate the data points and smooth to obtain the representation of the layer surface. Thickness data as used herein may include such data points as determined from the thickness or depth measurements and relationships from which segmentation and mapping is accomplished.

The post-refinement processing 920 may further include determining the thickness of each layer by measuring the short distance between its interfaces. Defining the thickness of the layer as the shortest distance between each two successive microlayers may be more accurate that defining thickness as a distance measured across the normal to a surface.

After post-segmentation processing 920, the imaging system may create three-dimensional maps as described herein at process 922. The three-dimensional maps may include one or more thickness, hyper-reflectivity, or irregularity maps. The three-dimensional maps may also be applied to a mapping scheme such as a heat map or bullseye map. The imaging system may display the generated three-dimensional map on a display screen. For example, heat maps may be generated that depict variations in thickness, hyper-reflectivity, or irregularities as different colors.

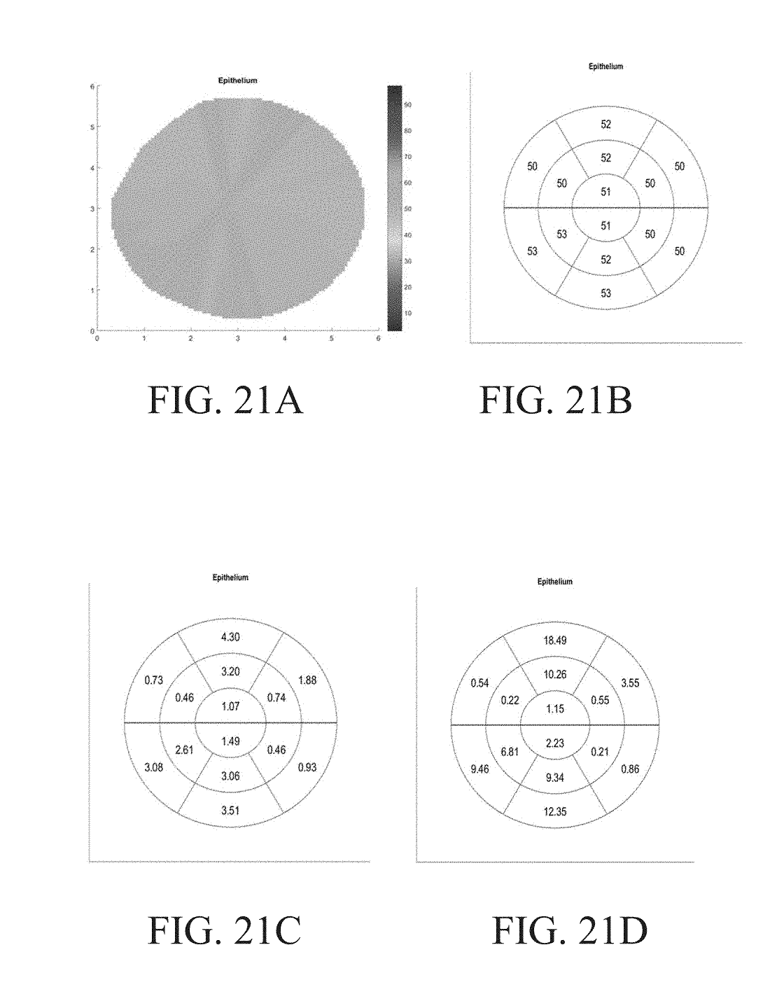

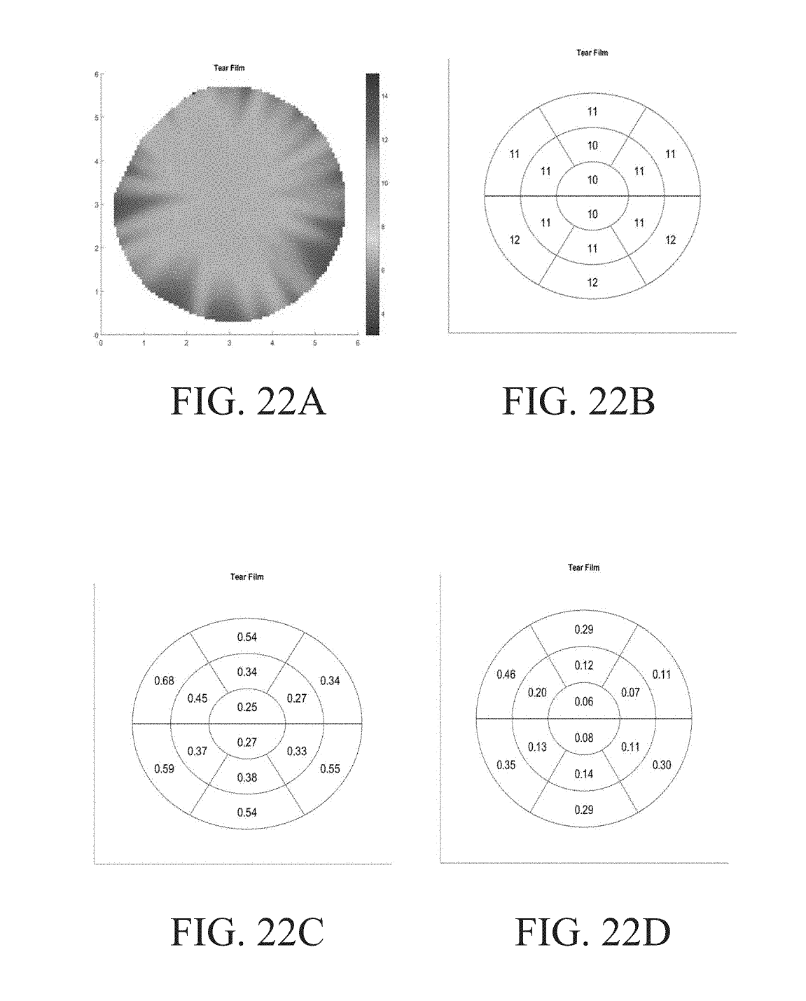

The imaging system may detect and analyze irregularities of the tear film, epithelium, or other layer using different processes. For example, FIG. 21A illustrates a heat map that depicts epithelium thickness. FIG. 22A illustrates a heat map that depicts tear film thickness. In some embodiments, heat map legends may be based on normative thickness data of the corneal tear film, epithelium, or other layer such that deviations for normative thickness is identified by designated coloring. FIGS. 21B-21D and FIGS. 22B-22D illustrate further examples of generated bullseye maps that depict the image data obtained from the segmentation and three-dimensional assembly to detect and analyze irregularities of the tear film, epithelium, or other layer, respectively, using different processes. For example, the imaging system may generate maps depicting regional data calculations, such as calculations corresponding the mean (FIG. 21B), standard deviation (FIG. 21C), or variance (FIG. 21D) of the epithelium layer or the mean (FIG. 22B), standard deviation (FIG. 22C), or variance (FIG. 22D) of the tear film layer.

As introduced above, to highlight the irregularities of the anterior surface of the ocular surface in isolation of the posterior surface of the layer which could get affected by other conditions, the imaging system may detect pixel differences between the segmented surface of the epithelium and a smooth curve that is created to model the ocular surface. For example, that are presented as heat and bullseye maps. Smooth model may be specifically generated with respect to the eye being examined. The smooth surface preferably corresponds to an idealized smooth curved surface corresponding to the generalized curved dimensions of the eye being examined, e.g., a smooth curved surface that matches the general curvature of the eye. Any suitable method of generating a smooth model surface may be used. For example, the model may be generated based on one or more sets of radial curvature data. A function that approximates a continuous smooth surface with respect to the particular eye being examined may be used. Generating the smooth model may include fitting radial curvature data to second, third, or forth order polynomials to represent a smooth curved surface representing an idealized smooth curved surface for the generalized dimensions of the eye being examined from which to compare layers identified in the segmented images or three-dimensional maps generated from the image data.

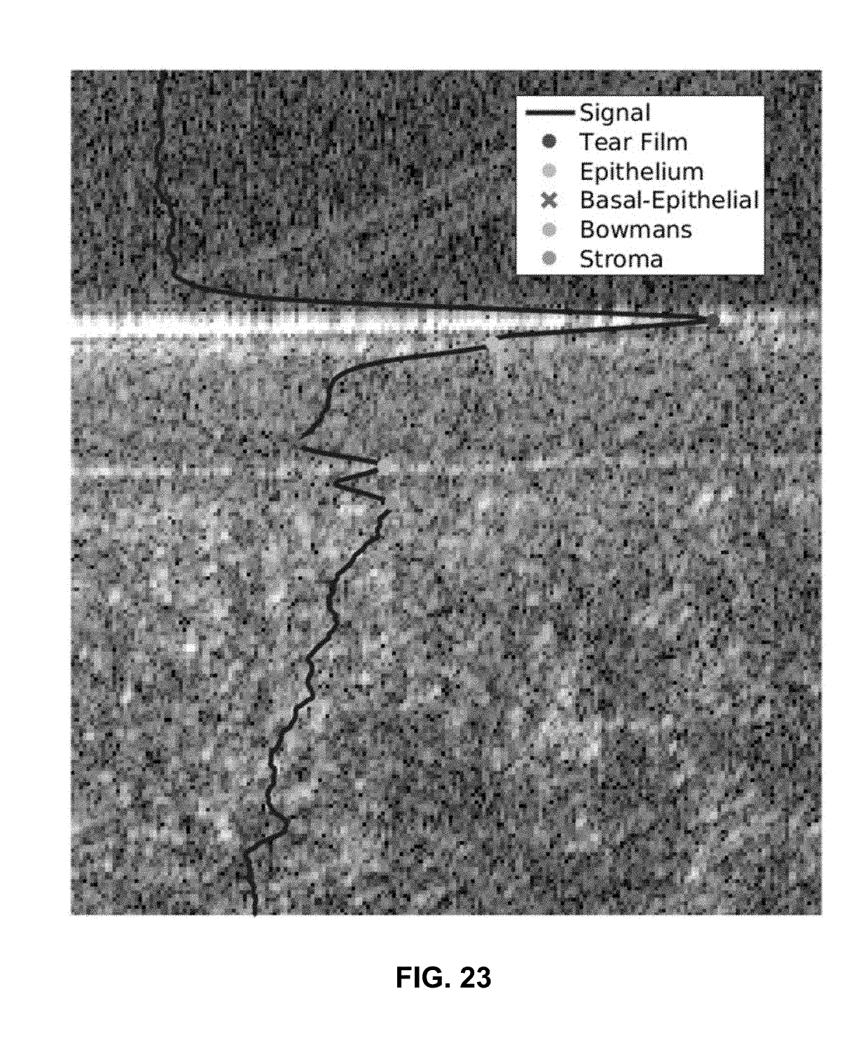

According to various embodiments, the imaging system may automatically detect the tear film and the epithelium as two separate layers to unmask the epithelial surface characteristics including the irregularities of the ocular surface, even when using an imaging device with relatively lower resolution. The imaging system may, for example, be configured to segment the epithelium layer separate of the tear film layer using lower resolution imaging, e.g., imaging having lower resolution than high or ultra-high-resolution images of the ocular surface. It is believed that, based on observation of ocular surface images, tears accumulate in areas of epithelial irregularities. This has been seen clinically using fluorescein staining of the tears under slit lamp magnification. In those areas, the distance between the tear film, epithelium, or other layer becomes larger and is more easily detected. In various embodiments, the imaging system may segment images obtained from machines with resolutions not sufficient to otherwise resolve the tear film from the epithelium, those areas of epithelial irregularities may be identified in OCT images as areas with a thicker and more hyper-reflective anterior-most white band. FIG. 23 illustrates an example vertical projection of an A-scan portion of the image (i.e., the blue-colored signal) is overlaid showing limited separation of the tear film and epithelium corresponding to a thickened white band having increased reflectivity. The white band is considered to identify combined epithelium and tear film surfaces. In particular, the hyper-reflectivity is considered to correspond to the tear film and fluid puddles or depressions/irregularities in the anterior surface of the epithelium. According to various embodiments, the imaging system may identify this transition and measure and quantify the thickness of this anterior-most band (e.g., along A-scan segments of the image) to segment the epithelium and tear film. Such image data may further translate to thickness data from which the layers may be mapped and further applied to three-dimensional mapping schemes. For example, the imaging system may generate a hyper-reflectivity map by combining reflectivity or thickness data obtained from multiple segmented images corresponding to the ocular surface of the cornea. The hyper-reflectivity map may identify relative reflectivity along the ocular surface thereby identifying topological irregularities in the epithelium, for example. Thus, the imaging system may segment the epithelium and tear film using vertical projection from a flattened image using the anterior surface or using hyper-reflectivity profile along the image. The imaging system may then generate thickness, irregularity, or hyper-reflectivity maps, as described above, depicting and quantifying this condition. As also shown in FIG. 23, boundaries between one or more other microlayers (or the beginning or end of a given microlayer) may be indicated by one or more peaks or valleys of the A-scan signal projected onto the image of the cornea. Additionally, or alternatively, as shown in FIG. 23, one or more other white bands (from the reflectivity data or the hyper-reflectivity map) may indicate boundaries between one or more other microlayers (or the beginning or end of a given microlayer) of the cornea. As such, the vertical projection or the reflectivity data may be used for segmentation or thickness determination of the microlayers of the cornea.

In a further example, the imaging system may also include a method to enhance mapping of the epithelial surface and epithelial irregularities, which may also be beneficially applied to images obtained from an imaging device with relatively lower resolution. For example, an eye drop may be used to augment the separation of the tear film and the anterior surface of the epithelium to artificially enlarge the tear film and thus separate it from the epithelium. This technique allows for clearer border of the epithelium and thus improved detection of the true surface. To achieve that, an eye drop may be instilled in the eye of the patient, and images are then taken. The epithelial surface may then become more clearly separable from the tear film on images of the ocular surface. Such a technique may be utilized to reveal the true surface of the epithelium that would be otherwise masked by the tear film and not detectable by a lower resolution device.

In various embodiments, the imaging system may provide a user with a plurality of selectable parameters from which to the system may use to generate three-dimensional maps. For example, the imaging system may include selections for thickness; hyper-reflectively; irregularity; comparative analytics with respect to multiple regions of the eye, normative data, or previously obtained data or maps thereof; statistical analytics such as mean, range, max, min, standard deviation, or variance; indices; or combinations thereof. The imaging system may also provide the user with a selection of mapping schema from which the imaging system is to generate the three-dimensional map, such as a thickness, heat, or bullseye map.

In various embodiments, the imaging system may be configured to analyze images or segmentation of such images to determine if the epithelium layer may be automatically segmented separate from the tear film. It has been observed by the inventors that certain individuals display significant separation between the epithelium and tear film layers sufficient to provide adequate segmentation even with lower resolution imaging devices. Thus, in some embodiments, the imaging system may apply a threshold separation distance, measured contrast parameter threshold between identified layers, or a threshold pixel count between estimated layers that is available for analysis in the images. In a further embodiment, the imaging system may compare segmentation via flattening of the anterior surface with that of segmentation base on a hyper-reflective anterior band to calculate whether a consensus threshold has been met. In various embodiments, imaging system may segment the epithelium and tear film layers together with images fall outside the threshold. In some embodiments, the imaging system may display a prompt to a user that resolution or the images are insufficient to separate the epithelium and tear film. In one such example, the user may be asked to provide new images, augment the eye with drops, or if the imaging system is to segment using the anterior white band, as described above. In one embodiment, the imaging system may automatically default to segmentation utilizing hyper-reflectivity of an anterior band when the system determines images fall outside the predefined threshold. In some embodiments, the imaging system may allow the user to define thresholds, which may be presented in a list. In one embodiment, the imaging system may include a mode in which the user may select that segmentation of the epithelium and tear film is to be performed based on a hyper-reflective band.

Mapping of the ocular surface (e.g., including the tear film) provides a new tool for diagnosing, treating, and testing of ocular conditions. For example, the mapping techniques described herein may be utilized by the imaging system to calculate the volume of the tear film and its distribution along the ocular surface. Such capabilities will be instrumental, for example, in testing the efficacy of dry eye therapies. In one embodiment, the imaging system may be utilized to detect the effect of a treatment (e.g., eye drops) on characteristics of the tear film, the epithelium, or other layer. For example, the imaging system may generate maps as described herein incorporating imagine data corresponding to characteristics such as thickness, hyper-reflectivity, shape, or volume. The data and maps may be analyzed for treatment response. For example, multiple characteristics may be analyzed for correlation of effects. One or more characteristics may also be tracked over time, which may include tracking correlation of multiple characteristics over time. In some embodiments, the imaging system may be configured to compare maps or image data thereof with maps or characteristic data of normal or affected eyes, standard or desired treatment response with respect to normal or affected eyes, or those of the subject eye before during or after a treatment regimen. As an example, the comparison may be presented in a three-dimensional map generated by the imaging system as described herein, e.g., color coding may represent correspondence or divergence in one or more regions of the ocular surface with respect to the comparison data or map. Accordingly, the imaging system may be utilized to evaluate treatments to provide analysis with respect to whether a treatment would enhance or change the characteristic of the tear film (such as thickness, hyper-reflectivity, shape, or volume) and for how long this effect may be expected to be retained, which could further be based on response of others having one or more similar characteristics prior to, during, or after treatment.

In various embodiments, the imaging system may be configured to detect change in characteristics of the tear film, epithelium, or other layer in response to a pharmacological treatment or therapy. For example, the imaging system may detect changes in thickness or volume as an effect of treatment by a pharmacological treatment or therapy, e.g., eye drops, used to treat dry eye syndrome. In some embodiments, the imaging system may calculate volume of the tear film by multiplying the thickness of the layer by the surface area of the ocular surface. The imaging system may also be configured to generate a map showing distribution of tear film volume along the corneal surface. Such a map may be presented in a heat or bullseye map that visually depicts thickness or volume distribution as raw or relative measurements (e.g., relative to other regions, normal or dry eye conditions, previous measurements, etc.).

In some embodiments, the imaging system may comprise an imaging device (e.g., a high-definition OCT imaging device or other imaging device). The imaging system may adjust a reference arm of the imaging device and use the adjusted imagine device to obtain one or more images of a cornea (e.g., high-resolution images of the cornea). As an example, the imaging system may adjust a reference arm of the imagine device to position the zero delay line (e.g., a point of maximum sensitivity on the imaging device) posterior to the cornea. In some embodiments, the cornea may be sealed within a container (e.g., a container filled with McCarey-Kaufman medium to sustain the cornea), and the imaging system may obtain the images of the cornea from outside the container. In one use case, based on the adjustment of the reference arm, one or more inverted images of the cornea may be obtained, where the anterior cornea is at the bottom of the images, and the posterior cornea is at the top of the images, thereby allowing for clear identification of the Endothelium/Descemet's membrane (En/DM) complex. In a further use case, the imaging system may perform one or more B-scans to obtain the inverted images of the cornea (e.g., that are through the center of the cornea) based on the adjustment of the reference arm.

In some embodiments, to compensate for the cornea being sealed with McCarey-Kaufman medium (or other medium) inside the container, the image system may be configured to match the dispersion between the reference arm and the sample arm (e.g., to achieve optimal axial resolution of the images). In some embodiment, approximate dispersion compensation can be performed by calculating the second and third order dispersion coefficients of the ocular components of interest. The coefficients may be tuned until the high image quality is reached. As an example, the imaging system may use numerical dispersion compensation techniques to automatically determine the optimal dispersion coefficients.

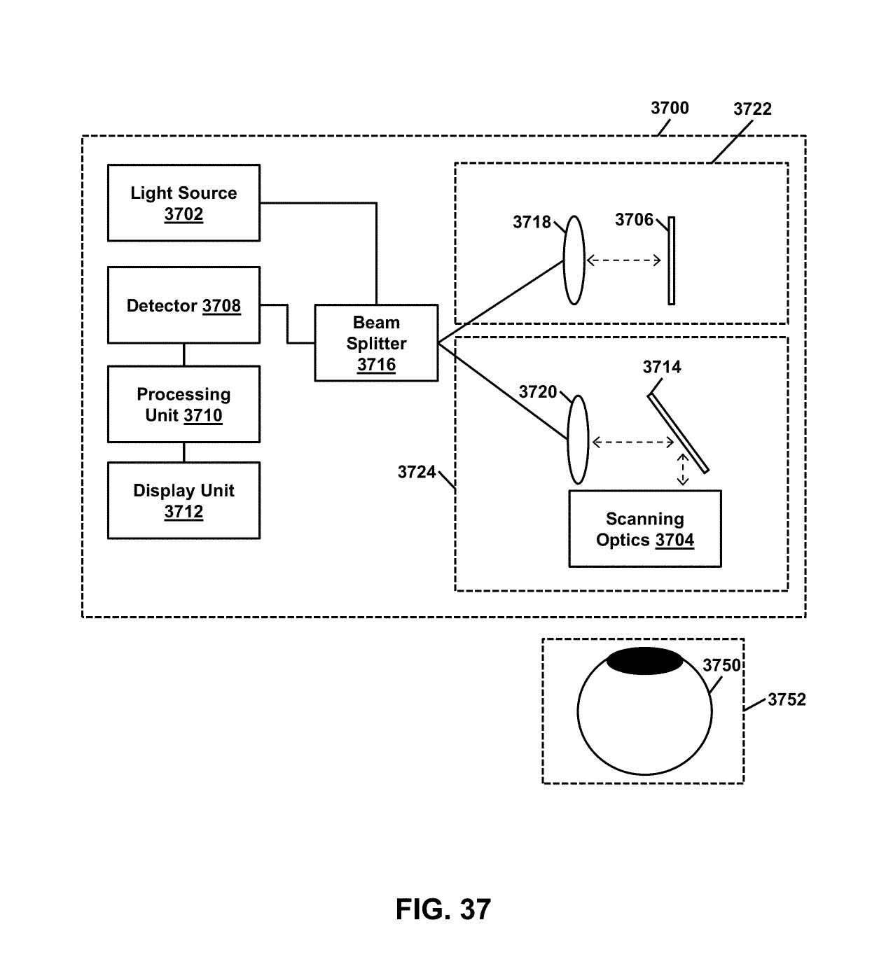

With respect to FIG. 37, in some embodiments, the imaging device (or system) 3700 may comprise a light source 3702 (e.g., low coherent light source, wavelength tunable laser source, or other light source), a scanning optic 3704, a reference mirror 3706, a detector 3708 (e.g., optical signal detector or other detector), a processing unit 3710, a display unit 3712, a scanning mirror 3714, a beam splitter 3716, lens 3718 and 3720, or other components. In some embodiments, at least one optical fiber coupler of the imaging device 3700 may be used to guide light from the light source 3702 illuminate a cornea 3750 (e.g., a human eye or other physical object). In one use case, the scanning optic 3704 (between the output of the optical fiber coupler and the cornea 3750) may scan the light so that a beam of light guided for the cornea 3750 is scanned laterally (in x-axis and/or y-axis) over the area or volume to be imaged. The scanning optic 3704 may comprise any optical element suitable for scanning. Light scattered from the cornea 3750 may be collected into the optical fiber coupler (e.g., that was used to guide the light for the illumination of the cornea 3750). In some embodiments, the beam splitter 3716 is configured to split and guide the light provided by the light source 3702 to a reference arm 3722 and a sampling arm 3724. In some embodiments, the imaging device 3700 may comprise the lens 3718 placed between the beam splitter 3716 and the retro-reflector 3706, and the lens 3720 placed between the beam splitter 3716 and the scanning optic 3704. As shown, in some embodiments, one or more images of the cornea 3750 may be obtained via the imaging device 3700 while the cornea 3750 is in a container 3752.

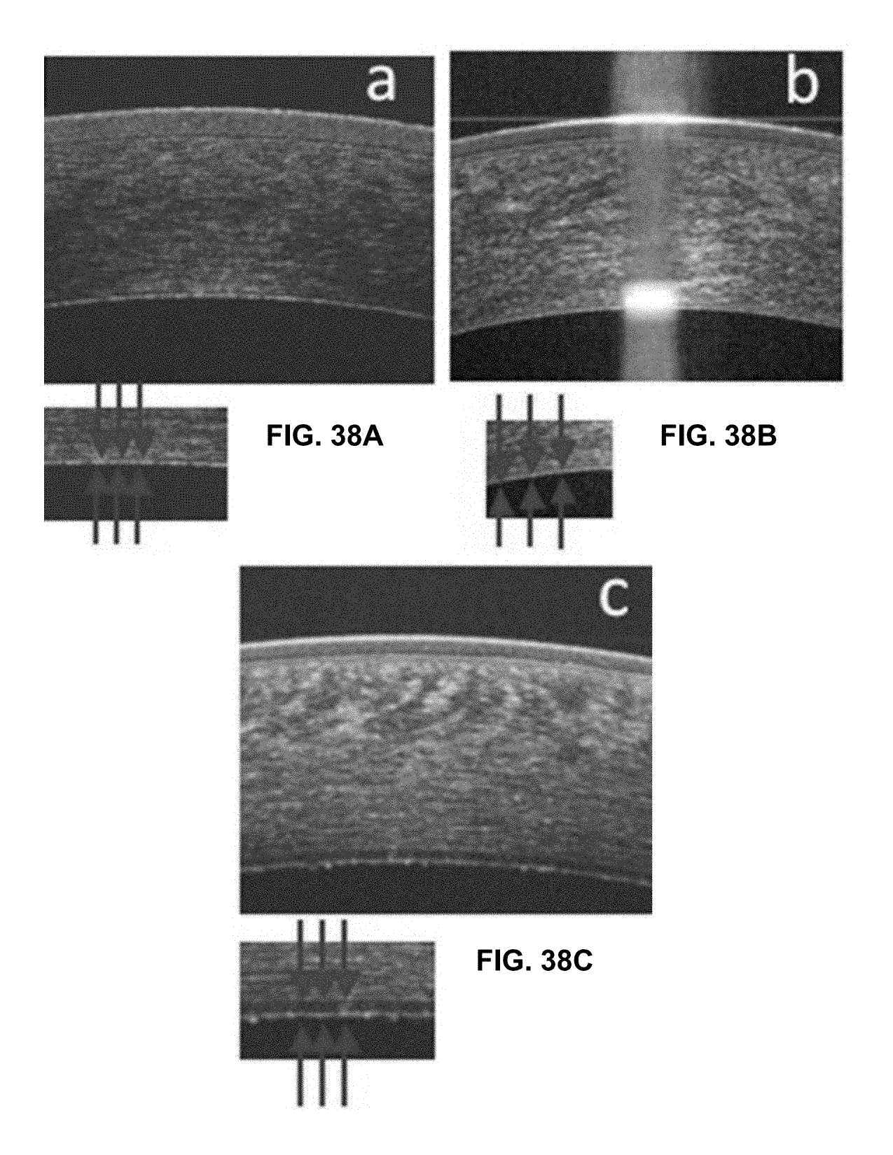

In some embodiments, the image system may perform segmentation on the images (e.g., images of a container-sealed donor cornea) to automatically deconstruct the corneal image into one or more microlayers via one or more segmentation techniques described herein (e.g., detection of the microlayer boundaries, registering and averaging of frames, etc.). Additionally, or alternatively, one or more thickness maps may be generated based on the segmented microlayers or via other techniques described herein (e.g., use of reflectivity data to determine thickness of the respective microlayers). As an example, FIGS. 38A, 38B, and 38C show a HD-OCT image from a donor graft, a HD-OCT image from a control eye (e.g., a normal eye), and a HD-OCT image from a Fuchs' endothelial corneal dystrophy eye. Each of the HD-OCT images is the result of segmentation showing the isolated En/DM complex demarcated with red arrows. Images are displayed with the zero delay at the bottom of the images. In this way, with respect to donor corneas, the segmented microlayers and the thickness maps may be used to detect corneal conditions (e.g., keratoconus, Fuchs' dystrophy, etc.) of the donor corneas while the donor corneas are in a sterile container prior to the donor corneas being transplanted into patients.

In one study, a custom built, high speed HD-OCT device was used to obtain corneal images. The light source was a 3-module superluminescent diode. The axial resolution of the imaging system was 3 μm in tissue. HD-OCT imaging was used to scan through the sealed sterile container of donor corneas stored in McCarey-Kaufman medium to image their En/DM complex. The system imaging was used in enhanced depth imaging (EDI) configuration to obtain images of the posterior cornea with high contrast. EDI HD-OCT images of the En/DM complex were obtained by adjusting the reference arm of the OCT system to position the zero delay line posterior to the cornea. With this configuration, an inverted image of the cornea was produced where the anterior cornea was at the bottom of the image and the posterior cornea at the top, allowing for clearer identification of the En/DM complex. The imaging system was used to obtain 3 mm×3 mm B-scan images (e.g., 15 frames per B-scan to improve signal strength) through the center of the donor corneas. Customized graph-based segmentation software was used to automatically deconstruct the corneal image into micro-layers based on edge/boundary detection, and frames were registered and averaged. The En/DM region was then segmented to produce En/DM thickness data. In the foregoing study, HD-OCT images of 20 control eyes from 20 patients were also captured and used to obtain in vivo normal En/DM thickness data. As proof of concept, 11 eyes of 7 patients with Fuchs' endothelial corneal dystrophy were imaged for comparison. The difference between donor cornea En/DM thickness and that of control subjects (16±2 μm) was not statistically significant (p=0.3). The difference between donor cornea En/DM thickness and that of Fuchs' endothelial corneal dystrophy eyes (25±5 μm) was statistically significant (p<0.05). The difference between the En/DM complex in control subjects and Fuchs' eyes was also statistically significant (p<0.05). Thus, the study demonstrated that EDI HD-OCT can be used to measure thickness of the En/DM complex of donor corneas stored in McCarey-Kaufman medium through a sealed sterile container. DM thickening is present in Fuchs' endothelial corneal dystrophy, posterior polymorphous corneal dystrophy, and corneal graft rejection. As such, such thickening may be used to assess the overall health of the En/DM tissue.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain operations may be distributed among the one or more processors, not only residing within a single machine, but also deployed across a number of machines. In some example embodiments, the processor or processors may be located in a single location (e.g., within a home environment, an office environment, or as a server farm), while in other embodiments the processors may be distributed across a number of locations.

The performance of certain operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Although the present invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the scope of the appended claims. For example, it is to be understood that the present invention contemplates that, to the extent possible, one or more features of any embodiment can be combined with one or more features of any other embodiment.

The present techniques will be better understood with reference to the following enumerated embodiments:

1. A method comprising: segmenting an image of a cornea; determining one or more characteristics of one or more layers of the cornea based on the segmentation of the image of the cornea; and generating a map for the one or more layers of the cornea based on the one or more characteristics.
2. The method of embodiment 1, wherein the one or more layers comprises an epithelium layer, a tear film layer, a basal epithelial layer, a Bowman's layer, or a Descemet's layer.
3. The method of any of embodiments 1-2, further comprising: performing an anterior surface registration or a posterior surface registration on each of a plurality of high-resolution cross-section images of the cornea to generate the image of the cornea, the image of the cornea being a high-resolution composite cross-section image of the cornea.
4. The method of any of embodiments 2-3, wherein each of the plurality of high-resolution images is captured when a subject is looking in a different direction than a direction the subject was looking when at least another one of the plurality of high-resolution images is captured.
5. The method of any of embodiments 1-4, wherein the map comprises a thickness map, irregularity map, or hyper-reflectivity map correlated to a diagnosable condition of the cornea.
6. The method of any of embodiments 1-5, wherein determining the one or more characteristics comprises determining thickness of the one or more layers of the cornea based on the segmentation of the image of the cornea, and wherein generating the map comprises generating a thickness map based on the thickness of the one or more layers of the cornea, the thickness map comprising visual differences in thickness across the one or more layers of the cornea.
7. The method of any of embodiments 1-6, wherein segmenting the image comprises segmenting the image of the cornea based on a vertical projection of the image of the cornea.
8. The method of any of embodiments 1-7, further comprising: determining an anterior surface in the image of the cornea; and flattening the image of the cornea based on the anterior surface; and segmenting the image of the cornea based on the vertical projection of the flattened image of the cornea.
9. The method of any of embodiments 7-8, further comprising: determining one or more contrast transition surfaces of the one or more layers of the cornea based on the vertical projection; and segmenting the image of the cornea based on the one or more contrast transition surfaces.
10. The method of embodiment 9, wherein the one or more contrast transition surfaces are determined based on a peak detection or a gradient algorithm.
11. The method of any embodiments 1-7, wherein segmenting the image of the cornea comprises segmenting the image of the cornea without flattening the image of the cornea.
12. The method of any of embodiments 1-11, wherein obtaining the image of the cornea comprises obtaining the image of the cornea via an imaging device outside a container while the cornea is sealed within the container.
13. The method of any of embodiments 1-12, further comprising: adjusting a reference arm of an imaging device to position a zero delay line posterior to the cornea; and obtaining the image of the cornea via the imaging device based on the adjustment of the reference arm of the imaging device.
14. The method of any of embodiments 1-13, wherein obtaining the image of the cornea comprises obtaining a B-scan image of the cornea.
15. The method of any of embodiments 1-14, wherein generating the map comprises assemble a cloud map of the one or more layers by aligning data points corresponding to the segmentation of the one or more layers in at least one section of the cornea with data points corresponding to segmentation of layers in images of additional sections of the cornea.
16. The method of any of embodiments 1-15, further comprising: refining the segmentation of the image of the cornea to more closely match the one or more layers of the cornea based on graph search theory (GS) or Randomized Hough Transform (RHT) technique; and determining the one or more characteristics of the one or more layers of the cornea based on the refined segmentation.
17. The method of any of embodiments 1-16, wherein the map depicts irregularities along an anterior surface of an epithelium layer of the cornea.
18. The method of embodiment 17, wherein the map comprises an irregularity map that depicts the irregularities in a heat map of the anterior surface of the epithelium layer.
19. The method of any of embodiments 1-18, further comprising: generating irregularity data by detecting pixel differences between an anterior surface of the one or more layers of the cornea and a smooth curved surface representative of a reference layer (e.g., an idealized epithelium layer or other reference layer) corresponding to general dimensions of the cornea determined from the image of the cornea.
20. The method of any of embodiments 1-19, further comprising: generating reflectivity data comprising ocular surface reflectivity related to the one or more layers in the image of the cornea; and segmenting the image of the cornea based on the reflectivity data.
21. The method of any of embodiments 1-20, further comprising: obtaining reflectivity data from the images of a plurality of sections of the cornea, the reflectivity data comprising an anterior white band in the images, the anterior white band comprising thickened hyper-reflective areas corresponding to anterior epithelial anterior surface irregularities; and generating, based on the reflectivity data, thickness data indicating thickness of the anterior white band; and segmenting the image of the cornea images based on the thickness data.
22. The method of embodiment 21, wherein the cornea is instilled with a fluid prior to capture of the image or the images such that separation between an epithelium layer and a tear film layer of the cornea is increased and the image or images reflect the increased separation.
23. The method of any of embodiments 1-22, further comprising: determining tear film volume across the cornea or ocular surface of the cornea based on the segmentation of the image of the cornea, wherein the map depicts tear volume across the cornea or the ocular surface of the cornea.
24. The method of embodiment 23, wherein the map comprises a bullseye map or heat map depicting differences in local tear volume across the cornea or the ocular surface of the cornea.
25. The method of any of embodiments 1-24, wherein the map comprises: (i) a bullseye map depicting mean, variance, or standard deviation of thickness across the tear film layer or epithelium layer, (ii) a bullseye map or heat map of a ratio or comparison of thickness among regions of the epithelium layer or tear film layer, (iii) a bullseye map or heat map of a ratio or comparison of thickness of the epithelium layer or tear film layer to normative data, or (iv) a bullseye map or heat map of a ratio or comparison of thickness of the epithelium layer or tear film layer to a diagnosable condition.
26. The method of any of embodiments 1-25, wherein the map comprises a thickness map of an epithelium layer of the cornea (or one or more other microlayers of the cornea), wherein the thickness map includes an irregularity indication of changes in thickness across the epithelium layer (or the other microlayers), and wherein the irregularity indication indicates differences in concentration of thickness irregularities across different regions of the epithelium layer (or the other microlayers).
27. The method of embodiment 26, wherein the different regions of the epithelium layer (or the other microlayers) comprise (i) a central portion of the cornea corresponding to a diagnosable condition being aqueous deficiency or (ii) a lower or upper portion of the cornea corresponding to a diagnosable condition being lipid deficiency dry eye syndrome or Meibomian gland dysfunction.
28. The method of any of embodiments 1-27, further comprising: determining an effect of a treatment on the one or more characteristics of the one or more layers of the cornea based on the map.
29. The method of embodiment 28, wherein the one or more characteristics comprise thickness, hyper-reflectivity, shape, or volume.
30. The method of any of embodiments 28-29, wherein the treatment comprises use of eye drops.
31. The method of any of embodiments 28-30, wherein determining the effect of the treatment comprises (i) determining a duration of the effect of the treatment on the one or more characteristics of the one or more layers of the cornea based on the map or (ii) determining a change of the one or more characteristics of the one or more layers of the cornea based on the map as at least one effect of the treatment.
32. A method comprising: obtaining reflectivity data from high-resolution images of a plurality of sections of a cornea, the reflectivity data including an anterior white band in the high-resolution images, and the anterior white band comprising thickened hyper-reflective areas corresponding to anterior epithelial anterior surface irregularities; measuring the reflectivity data and quantify thickness of the anterior white band in the high-resolution images to generate thickness data (e.g., indicating thickness of each of the tear film layer and epithelium layer); assembling a cloud map of the epithelium layer or the tear film layer by aligning data points corresponding to the tear film layer or the epithelium layer based on the thickness data; and generating a map of the epithelium layer or tear film layer.
33. The method of embodiment 32, wherein the map comprises a hyper-reflectivity map.
34. The method of embodiment 33, wherein the hyper-reflectivity map depicts relative reflectivity, and wherein areas of increased relative reflectivity correspond to areas of depression along the anterior surface of the epithelium layer.
35. The method of any of embodiments 32-34, further comprising: determining an effect of a treatment on one or more characteristics of the tear film layer or the epithelium layer based on the map.
36. The method of embodiment 35, wherein the one or more characteristics comprise thickness, hyper-reflectivity, shape, or volume.
37. The method of any of embodiments 35-36, wherein the treatment comprises use of eye drops.
38. The method of any of embodiments 35-37, wherein determining the effect of the treatment comprises (i) determining a duration of the effect of the treatment on the one or more characteristics of the tear film layer or the epithelium layer based on the map or (ii) determining a change of the one or more characteristics of the tear film layer or the epithelium layer based on the map as at least one effect of the treatment.
39. A tangible, non-transitory, machine-readable medium storing instructions that, when executed by a data processing apparatus, cause the data processing apparatus to perform operations comprising those of any of embodiments 1-38.
40. A system comprising: one or more processors; and memory storing instructions that, when executed by the processors, cause the processors to effectuate operations comprising those of any of embodiments 1-38.

What is claimed is:

1. A system comprising:
a computer system that comprises one or more processors programmed with computer program instructions that, when executed, cause the computer system to:
obtain high-resolution images of a cornea;
segment the high-resolution images of the cornea to detect a tear film layer of the cornea and an epithelium layer of the cornea;
determining thickness of the tear film layer and thickness of the epithelium layer based on the segmentation of the high-resolution images of the cornea; and
generating a thickness map based on the thickness of the tear film layer and the thickness of the epithelium layer, the thickness map comprising visual differences in thickness across the tear film layer and the epithelium layer.

2. The system of claim 1, wherein segmenting the high-resolution images of the cornea comprises segmenting the high-resolution images of the cornea without flattening the high-resolution images of the cornea.

3. The system of claim 1, wherein segmenting the high-resolution images of the cornea comprises segmenting the high-resolution images of the cornea based on one or more vertical projections of the high-resolution images of the cornea.

4. The system of claim 1, wherein obtaining the high-resolution images of the cornea comprises obtaining the high-resolution images of the cornea via an imaging device outside a container while the cornea is sealed within the container.

5. The system of claim 4, wherein the computer system is caused to:
adjust a reference arm of the imaging device to position a zero delay line posterior to the cornea; and
obtain the high-resolution images of the cornea via the imaging device based on the adjustment of the reference arm of the imaging device.

6. The system of claim 4, wherein obtaining the high-resolution images of the cornea comprises obtaining B-scan images of the cornea via the imaging device outside the container while the cornea is sealed within the container.

7. The system of claim 1, wherein the computer system is caused to:
determine an anterior surface in the high-resolution images of the cornea;
flatten the high-resolution images of the cornea based on the anterior surface; and
segment the high-resolution images of the cornea based on a vertical projection of the flattened image of the cornea.

8. The system of claim 1, wherein the thickness map comprises at least one of: (i) a bullseye or heat map depicting thickness across the tear film layer and the epithelium layer, (ii) a bullseye map or heat map of a ratio or comparison of thickness among regions of the epithelium layer or tear film layer, (iii) a bullseye map or heat map of a ratio or comparison of thickness of the epithelium layer or tear film layer to normative data, or (iv) a bullseye map or heat map of a ratio or comparison of thickness of the epithelium layer or tear film layer to a diagnosable condition.

9. A method implemented by a computer system that comprises one or more processors executing computer program instructions that, when executed, perform the method, the method comprising:
obtaining an image of a cornea;
segmenting the image of the cornea without flattening the image of the cornea to detect one or more microlayers of the cornea;
determining thickness of the one or more microlayers of the cornea based on the segmentation of the image of the cornea; and
generating a thickness map for the one or more microlayers of the cornea based on the thickness of the one or more microlayers of the cornea, the thickness map comprising visual differences in thickness across the one or more microlayers of the cornea.

10. The method of claim 9, wherein obtaining the image of the cornea comprises obtaining the image of the cornea via an imaging device outside a container while the cornea is sealed within the container.

11. The method of claim 10, further comprising:
adjust a reference arm of the imaging device to position a zero delay line posterior to the cornea; and
obtain the image of the cornea via the imaging device based on the adjustment of the reference arm of the imaging device.

12. The method of claim 13, wherein obtaining the image of the cornea comprises obtaining a B-scan image of the cornea via the imaging device outside the container while the cornea is sealed within the container.

13. The method of claim 9, wherein the thickness map comprises at least one of: (i) a bullseye or heat map depicting thickness across the one or more microlayers, (ii) a bullseye map or heat map of a ratio or comparison of thickness among regions of the one or more microlayers, (iii) a bullseye map or heat map of a ratio or comparison of thickness of the one or more microlayers to normative data, or (iv) a bullseye map or heat map of a ratio or comparison of thickness of the one or more microlayers to a diagnosable condition.

14. A system comprising:
a computer system that comprises one or more processors programmed with computer program instructions that, when executed, cause the computer system to:
obtain an image of a cornea;
segment the image of the cornea to detect one or more microlayers of the cornea;
determining thickness of the one or more microlayers of the cornea based on the segmentation of the image of the cornea; and
generating a thickness map for the one or more microlayers of the cornea based on the thickness of the one or more microlayers of the cornea, the thickness map comprising visual differences in thickness across the one or more microlayers of the cornea.

15. The system of claim 14, wherein segmenting the image of the cornea comprises segmenting the image of the cornea without flattening the image of the cornea.

16. The system of claim 14, wherein segmenting the image of the cornea comprises segmenting the image of the cornea based on a vertical projection of the image of the cornea.

17. The system of claim 14, wherein obtaining the image of the cornea comprises obtaining the image of the cornea via an imaging device outside a container while the cornea is sealed within the container.

18. The system of claim 17, wherein the computer system is caused to:
adjust a reference arm of the imaging device to position a zero delay line posterior to the cornea; and
obtain the image of the cornea via the imaging device based on the adjustment of the reference arm of the imaging device.

19. The system of claim 18, wherein obtaining the image of the cornea comprises obtaining a B-scan image of the cornea via the imaging device outside the container while the cornea is sealed within the container.

20. The system of claim 14, wherein the thickness map comprises at least one of: (i) a bullseye or heat map depicting thickness across the one or more microlayers, (ii) a bullseye map or heat map of a ratio or comparison of thickness among regions of the one or more microlayers, (iii) a bullseye map or heat map of a ratio or comparison of thickness of the one or more microlayers to normative data, or (iv) a bullseye map or heat map of a ratio or comparison of thickness of the one or more microlayers to a diagnosable condition.
